(19) United States
(12) Patent Application Publication
(10) Pub. No.: US 2007/0065820 A1
(43) Pub. Date: Mar. 22, 2007

(54) Lipid-associated molecules

(76) Inventors: Xin Jiang, Saratoga, CA (US); Shanya D. Becha, San Francisco, CA (US); Sean A. Bulloch, Anaheim, CA (US); Hsin-Ru Chang, Belmont, CA (US); Narinder K. Chawla, Union City, CA (US); Vicki S. Elliott, San Jose, CA (US); Brooke M. Emerling, Chicago, IL (US); Kimberly J. Gietzen, San Jose, CA (US); April J.A. Hafalia, Daly City, CA (US); Alan A. Jackson, Los Gatos, CA (US); Amy E. Kable, Silver Spring, MD (US); Reena Khare, Saratoga, CA (US); Soo Yeun Lee, Mountain View, CA (US); Joseph P. Marquis, San Jose, CA (US); Jagi Murage, San Jose, CA (US); Anita Swarnakar, San Francisco, CA (US); Yonghong G. Yang, San Jose, CA (US)

Correspondence Address: FOLEY AND LARDNER LLP;SUITE 500, 3000 K STREET NW, WASHINGTON, DC, 20007, US

(21) Appl. No.: 10/534,579

(22) PCT Filed: Nov. 10, 2003
(86) PCT No.: PCT/US03/35946

§ 371 Date: Nov. 14, 2006

Related U.S. Application Data
(60) Provisional application No. 60/426,105, filed on Nov. 13, 2002.
(60) Provisional application No. 60/433,215, filed on Dec. 12, 2002.
(60) Provisional application No. 60/453,127, filed on Mar. 7, 2003.
(60) Provisional application No. 60/454,801, filed on Mar. 13, 2003.
(60) Provisional application No. 60/465,619, filed on Apr. 24, 2003.
(60) Provisional application No. 60/465,495, filed on Apr. 24, 2003.
(60) Provisional application No. 60/491,800, filed on Aug. 1, 2003.

(51) Int. Cl. C12Q 1/68; C40B 30/06; C07K 14/775; A61K 38/17; G01N 33/53; C07H 21/04; C12P 21/06; C12P 21/04; C12N 1/21

(52) U.S. Cl. 435/6; 435/69.1; 435/320.1; 435/325; 530/359; 536/23.5; 435/252.3; 530/388.25; 514/12; 435/70.21; 435/7.1; 435/287.2

(57) ABSTRACT

Various embodiments of the invention provide human lipid-associated molecules (LIPAM) and polynucleotides which identify and encode LIPAM. Embodiments of the invention also provide expression vectors, host cells, antibodies, agonists, and antagonists. Other embodiments provide methods for diagnosing, treating, or preventing disorders associated with aberrant expression of LIPAM.

### TECHNICAL FIELD

The invention relates to novel nucleic acids, lipid-associated molecules encoded by these nucleic acids, and to the use of these nucleic acids and proteins in the diagnosis, treatment, and prevention of cancer, cardiovascular, neurological, autoimmune/inflammatory, and gastrointestinal disorders, and disorders of lipid metabolism. The invention also relates to the assessment of the effects of exogenous compounds on the expression of nucleic acids and lipid-associated molecules.

### BACKGROUND OF THE INVENTION

Lipids are water-insoluble, oily or greasy substances that are soluble in nonpolar solvents such as chloroform or ether. Neutral fats (triacylglycerols) serve as major fuels and energy stores. Fatty acids are long-chain organic acids with a single carboxyl group and a long non-polar hydrocarbon tail. Long-chain fatty acids are essential components of glycolipids, phospholipids, and cholesterol, which are building blocks for biological membranes, and of triglycerides, which are biological fuel molecules. Lipids, such as phospholipids, sphingolipids, glycolipids, and cholesterol, are key structural components of cell membranes. Lipids and proteins are associated in a variety of ways. Glycolipids form vesicles that carry proteins within cells and cell membranes. Interactions between lipids and proteins function in targeting proteins and glycolipids involved in a variety of processes, such as cell signaling and cell proliferation, to specific membrane and intracellular locations. Various proteins are associated with the biosynthesis, transport, and uptake of lipids. In addition, key proteins involved in signal transduction and protein targeting have lipid-derived groups added to them post-translationally (Stryer, L. (1995) Biochemistry, W.H. Freeman and Co., New York N.Y., pp. 264-267, 934; Lehninger, A. (1982) Principles of Biochemistry, Worth Publishers, Inc. New York N.Y.; and ExPASy “Biochemical Pathways” index of Boehringer Mannheim World Wide Web site, “http://www.expasy.ch/cgi-bin/search-biochem-index”.)

Phospholipids

A major class of phospholipids are the phosphoglycerides, which are composed of a glycerol backbone, two fatty acid chains, and a phosphorylated alcohol. Phosphoglycerides are components of cell membranes. Principal phosphoglycerides are phosphatidyl choline, phosphatidyl ethanolamine, phosphatidyl serine, phosphatidyl inositol, and diphosphatidyl glycerol. Many enzymes involved in phosphoglyceride synthesis are associated with membranes (Meyers, R. A. (1995) Molecular Biology and Biotechnology, VCH Publishers Inc., New York N.Y., pp. 494-501). Phosphatidate is converted to CDP-diacylglycerol by the enzyme phosphatidate cytidylyltransferase (ExPASy ENZYME EC 2.7.7.41). Transfer of the diacylglycerol group from CDP-diacylglycerol to serine to yield phosphatidyl serine, or to inositol to yield phosphatidyl inositol, is catalyzed by the enzymes CDP-diacylglycerol-serine O-phosphatidyltransferase and CDP-diacylglycerol-inositol 3-phosphatidyltransferase, respectively (ExPASy ENZYME EC 2.7.8.8; ExPASy ENZYME EC 2.7.8:11). The enzyme phosphatidyl serine decarboxylase catalyzes the conversion of phosphatidyl serine to phosphatidyl ethanolamine, using a pyruvate cofactor (Voelker, D. R. (1997) Biochim. Biophys. Acta 1348:236-244). Phosphatidyl choline is formed using diet-derived choline by the reaction of CDP-choline with 1,2-diacylglycerol, catalyzed by diacylglycerol cholinephosphotransferase (ExPASy ENZYME 2.7.8.2).

Other phosphoglycerides have been shown to be involved in the vesicle trafficking process. Phosphatidylinositol transfer protein (PITP) is a ubiquitous cytosolic protein, thought to be involved in transport of phospholipids from their site of synthesis in the endoplasmic reticulum and Golgi to other cell membranes. More recently, PITP has been shown to be an essential component of the polyphosphoinositide synthesis machinery and is hence required for proper signaling by epidermal growth factor and f-Met-Leu-Phe, as well as for exocytosis. The role of PITP in polyphosphoinositide synthesis may also explain its involvement in intracellular vesicular traffic (Liscovitch, M. et al. (1995) Cell 81:659-662).

The copines are phospholipid-binding proteins believed to function in membrane trafficking. Copines promote lipid vesicle aggregation. They contain a C2 domain associated with membrane activity and an annexin-type domain that mediates interactions between integral and extracellular proteins and is associated with calcium binding and regulation (Creutz, C. E. (1998) J. Biol. Chem. 273:1393-1402). Other C2-containing proteins include the synaptotagmins, a family of proteins involved in vesicular trafficking. Synaptotagmin concentrations in cerebrospinal fluid have been found to be reduced in early-onset Alzheimer's disease (Gottfries, C. G. et al. (1998) J. Neural Transm. 105:773-786).

The phosphatidylinositol-transfer protein Sec14, which catalyses exchange of phosphatidylinositol and phosphatidylcholine between membrane bilayers in vitro, is essential for vesicle budding from the Golgi complex. Sec14 includes a carboxy-terminal domain that forms a hydrophobic pocket which represents the phospholipid-binding domain. (Sha, B. et al. (1998) Nature 391:506-510). Sec14 is a member of the cellular retinaldehyde-binding protein (CRAL)/Triple function domain (TRIO) family (InterPro Entry IPR001251, http://www.ebi.ac.uk/interpro).

Sphingolipids

Sphingolipids are an important class of membrane lipids that contain sphingosine, a long chain amino alcohol. They are composed of one long-chain fatty acid, one polar head alcohol, and sphingosine or sphingosine derivatives. The three classes of sphingolipids are sphingomyelins, cerebrosides, and gangliosides. Sphingomyelins, which contain phosphocholine or phosphoethanolamine as their head group, are abundant in the myelin sheath surrounding nerve cells. Galactocerebrosides, which contain a glucose or galactose head group, are characteristic of the brain. Other cerebrosides are found in non-neural tissues. Gangliosides, whose head groups contain multiple sugar units, are abundant in the brain, but are also found in non-neural tissues.

Glycolipids

Glycolipids are also important components of the plasma membranes of animal cells. The most simple glycolipid is cerebroside which comprises only a single glucose or galactose sugar residue in addition to the lipid component. Gangliosides are glycosphingolipid plasma membrane components that are abundant in the nervous systems of vertebrates. Gangliosides are the most complex glycolipids and comprise ceramide (acylated sphingosine) attached to an oligosaccharide moiety containing at least one acidic sugar residue (sialic acid), namely N-acetylneuraminate or N-glycolylneuraminate. The sugar residues are added sequentially to ceramide via UDP-glucose, UDP-galactose, N-acetylgalactosamine, and CMP-N-acetylneuraminate donors. Over 15 gangliosides have been identified with G_{M1} and G_{M2} being the best characterized (Stryer, L (1988) Biochemistry, W.H Freeman and Co., Inc. New York. pp. 552-554).

Gangliosides are thought to play important roles in cell surface interactions, cell differentiation, neuritogenesis, the triggering and modulation of transmembrane signaling, mediatiosynaptic function, neural repair, neurite outgrowth, and neuronal death (Hasegawa, T. et al. (2000) J. Biol. Chem. 275:8007-8015). While the presence of gangliosides in the plasma membrane is important for orchestrating these events, the subsequent removal of carbohydrate groups (desialylation) by sialidases also appears to be important for regulating neuronal differentiation.

Specific soluble N-ethylmaleimide-sensitive factor attachment protein (SNAP) receptor (SNARE) proteins are required for different membrane transport steps. The SNARE protein Vti1a has been colocalized with Golgi markers while Vti1b has been colocalized with Golgi and the trans-Golgi network of endosomal markers in fibroblast cell lines. A brain-specific splice variant of Vti1a is enriched in small synaptic vesicles and clathrin-coated vesicles isolated from nerve terminals. Vti1a-beta and synaptobrevin are integral parts of synaptic vesicles throughout their life cycle. Vti1a-beta functions in a SNARE complex during recycling or biogenesis of synaptic vesicles (Antonin, W. et al. (2000) J. Neurosci. 20:5724-5732).

Sialidases catalyze the first step in glycosphingolipid degradation, removing carbohydrate moieties from gangliosides. These enzymes are present in the cytosol, lysosomal matrix, lysosomal membrane, and plasma membrane (Hasegawa, T. et al. (2000) J. Biol. Chem. 275:8007-8015). Hallmark features of sialidases include a transmembrane domain, an Arg-Ile-Pro domain, and three Asp-box sequences (Wada, T. (1999) Biochem. Biophys. Res. Commun. 261:21-27).

During normal neuronal development, pyramidal neurons of the cerebral cortex participate in a single burst of dendritic sprouting immediately following nerve cell migration to the cortical mantle. Cells undergoing dendritogenesis are characterized by increased expression of G_{M2} ganglioside which decreases following dentritic maturation. Evidence suggests that no new primary dendrites are initiated following the initial burst.

Cholesterol

Cholesterol, composed of four fused hydrocarbon rings with an alcohol at one end, moderates the fluidity of membranes in which it is incorporated. In addition, cholesterol is used in the synthesis of steroid hormones such as cortisol, progesterone, estrogen, and testosterone. Bile salts derived from cholesterol facilitate the digestion of lipids. Cholesterol in the skin forms a barrier-that prevents excess water evaporation from the body. Farnesyl and geranylgeranyl groups, which are derived from cholesterol biosynthesis intermediates, are post-translationally added to signal transduction proteins such as Ras and protein-targeting proteins such as Rab. These modifications are important for the activities of these proteins (Guyton, A. C. (1991) Textbook of Medical Physiology, W.B. Saunders Company, Philadelphia Pa., pp. 760-763; Stryer, supra, pp. 279-280, 691-702, 934).

Mammals obtain cholesterol derived from both de novo biosynthesis and the diet. The liver is the major site of cholesterol biosynthesis in mammals. Biosynthesis is accomplished via a series of enzymatic steps known as the mevalonate pathway. The rate-limiting step is the conversion of hydroxymethylglutaryl-Coenzyme A (EMG-CoA) to mevalonate by HMG-CoA reductase. The drug lovastatin, a potent inhibitor of HMG-CoA reductase, is given to patients to reduce their serum cholesterol levels. Cholesterol derived from de novo biosynthesis or from the diet is transported in the body fluids in the form of lipoprotein particles. These particles also transport triacylglycerols. The particles consist of a core of hydrophobic lipids surrounded by a shell of polar lipids and apolipoproteins. The protein components serve in the solubilization of hydrophobic lipids and also contain cell-targeting signals. Lipoproteins include chylomicrons, chylomicron remnants, very-low-density lipoproteins (VLDL), intermediate-density lipoproteins (IDL), low-density lipoproteins (LDL), and high-density lipoproteins (HDL) (Meyers, supra; Stryer, supra, pp. 691-702). There is a strong inverse correlation between the levels of plasma HDL and risk of premature coronary heart disease. ApoL is an HDL apolipoprotein expressed in the pancreas (Duchateau, P. N. et al. (1997) J. Biol. Chem. 272:25576-25582).

Most cells outside the liver and intestine take up cholesterol from the blood rather than synthesize it themselves. Cell surface LDL receptors bind LDL particles which are then internalized by endocytosis (Meyers, supra). Absence of the LDL receptor, the cause of the disease familial hypercholesterolemia, leads to increased plasma cholesterol levels and ultimately to atherosclerosis (Stryer, supra, pp. 691-702).

Proteins involved in cholesterol uptake and biosynthesis are tightly regulated in response to cellular cholesterol levels. The sterol regulatory element binding protein (SREBP) is a sterol-responsive transcription factor. Under normal cholesterol conditions, SREBP resides in the endoplasmic reticulum membrane. When cholesterol levels are low, a regulated cleavage of SREBP occurs which releases the extracellular domain of the protein. This cleaved domain is then transported to the nucleus where it activates the transcription of the LDL receptor gene, and genes encoding enzymes of cholesterol-synthesis, by binding the sterol regulatory element (SRE) upstream of the genes (Yang, J. et al. (1995) J. Biol. Chem. 270:12152-12161). Regulation of cholesterol uptake and biosynthesis also occurs via the oxysterol-binding protein (OSBP). Oxysterols are oxidation products formed during the catabolism of cholesterol, and are involved in regulation of steroid biosynthesis. OSBP is a high-affinity intracellular receptor for a variety of oxysterols that down-regulate cholesterol synthesis and stimulate cholesterol esterification (Lagace, T. A. et al. (1997) Biochem. J. 326:205-213).

Supernatant protein factor (SPF), which stimulates squalene epoxidation and conversion of squalene to lanosterol, is a cytosolic squalene transfer protein that enhances cholesterol biosynthesis. Squalene epoxidase, a membrane-associated enzyme that converts squalene to squalene 2,3-oxide, plays an important role in the maintenance of cholesterol homeostasis. SPF belongs to a family of cytosolic lipid-binding/transfer proteins such as alpha-tocopherol transfer protein, cellular retinal binding protein, yeast phosphatidylinositol transfer protein (Sec14p), and squid retinal binding protein (Shibata, N. et al. (2001) Proc. Natl. Acad. Sci. U.S.A. 98:2244-2249).

Lipid Metabolism Enzymes

Long-chain fatty acids are also substrates for eicosanoid production, and are important in the functional modification of certain complex carbohydrates and proteins. 16-carbon and 18-carbon fatty acids are the most common. Fatty acid synthesis occurs in the cytoplasm. In the first step, acetyl-Coenzyme A (CoA) carboxylase (ACC) synthesizes malonyl-CoA from acetyl-CoA and bicarbonate. The enzymes which catalyze the remaining reactions are covalently linked into a single polypeptide chain, referred to as the multifunctional enzyme fatty acid synthase (FAS). FAS catalyzes the synthesis of palmitate from acetyl-CoA and malonyl-CoA. FAS contains acetyl transferase, malonyl transferase, β-ketoacetyl synthase, acyl carrier protein, β-ketoacyl reductase, dehydratase, enoyl reductase, and thioesterase activities. The final product of the FAS reaction is the 16-carbon fatty acid palmitate. Further elongation, as well as unsaturation, of palmitate by accessory enzymes of the ER produces the variety of long chain fatty acids required by the individual cell. These enzymes include a NADH-cytochrome b₅ reductase, cytochrome b₅, and a desaturase.

Within cells, fatty acids are transported by cytoplasmic fatty acid binding proteins (Online Mendelian Inheritance in Man (OMIM) #134650 Fatty Acid-Binding Protein 1, Liver; FABP1). Diazepam binding inhibitor (DBI), also known as endozepine and acyl CoA-binding protein, is an endogenous γ-aminobutyric acid (GABA) receptor ligand which is thought to down-regulate the effects of GABA. DBI binds medium- and long-chain acyl-CoA esters with very high affinity and may function as an intracellular carrier of acyl-CoA esters (OMIM #125950 Diazepam Binding Inhibitor; DBI; PROSITE PDOC00686 Acyl-CoA-binding protein signature).

Fat stored in liver and adipose triglycerides may be released by hydrolysis and transported in the blood. Free fatty acids are transported in the blood by albumin. Triacylglycerols, also known as triglycerides and neutral fats, are major energy stores in animals. Triacylglycerols are esters of glycerol with three fatty acid chains. Glycerol-3-phosphate is produced from dihydroxyacetone phosphate by the enzyme glycerol phosphate dehydrogenase or from glycerol by glycerol kinase. Fatty acid-CoAs are produced from fatty acids by fatty acyl-CoA synthetases. Glyercol-3-phosphate is acylated with two fatty acyl-CoAs by the enzyme glycerol phosphate acyltransferase to give phosphatidate. Phosphatidate phosphatase converts phosphatidate to diacylglycerol, which is subsequently acylated to a triacylglyercol by the enzyme diglyceride acyltransferase. Phosphatidate phosphatase and diglyceride acyltransferase form a triacylglyerol synthetase complex bound to the ER membrane.

Dihydroxyacetone phosphate acyltransferase (DHAPAT), also known as glyceronephosphate O-acyltransferase (GNPAT), is a membrane-bound enzyme which catalyzes esterification of the free hydroxyl group of DHAP by long chain acyl CoA's to form acyl DHAP, the obligate precursor of glycerol ether lipids in animals which can also be converted to non-ether glycerolipids. DHAPAT is present in the peroxisomes of all animal cells examined except erythrocytes, but is not found in plant and bacteria cells. It is, however, present in Saccharomyces cerevisiae. With the exception of S. cerevisiae, it is found in close association in cellular membranes with other enzymes catalyzing the synthesis of ether lipid intermediates. The enzyme uses the CoA derivatives of palmitate, stearate, and oleate, with the highest activity on palmitoyl-CoA. It shows low activity towards mono- or polyunsaturated acyl CoA's. DHAPAT is lacking in several peroxisomal disorders including Zellweger cerebrohepatorenal syndrome and rhizomelic chondrodysplasia punctata (RCDP) type 2. RCDP type 2 causes severe developmental delay, cataracts, and shortening of the limbs. This DHAPAT deficiency leads to a decreased level of ether lipids in the cellular membrane. Moreover, studies question whether DHAPAT is also involved in the biosynthesis of non-ether lipids in animals, since there is high DHAPAT activity in low ether lipid-containing tissues, such as liver and adipose tissues (Ofman, R. et al. (1998) Hum. Mol. Genet. 7:847-853; Hajra, A. K. (1997) Biochim. Biophys. Acta 1348:27-34).

Mitochondrial and peroxisomal beta-oxidation enzymes degrade saturated and unsaturated fatty acids by sequential removal of two-carbon units from CoA-activated fatty acids. The main beta-oxidation pathway degrades both saturated and unsaturated fatty acids while the auxiliary pathway performs additional steps required for the degradation of unsaturated fatty acids. The pathways of mitochondrial and peroxisomal beta-oxidation use similar enzymes, but have different substrate specificities and functions. Mitochondria oxidize short-, medium-, and long-chain fatty acids to produce energy for cells. Mitochondrial beta-oxidation is a major energy source for cardiac and skeletal muscle. In liver, it provides ketone bodies to the peripheral circulation when glucose levels are low as in starvation, endurance exercise, and diabetes (Eaton, S. et al. (1996) Biochem. J. 320:345-357). Peroxisomes oxidize medium-, long-, and very-long-chain fatty acids, dicarboxylic fatty acids, branched fatty acids, prostaglandins, xenobiotics, and bile acid intermediates. The chief roles of peroxisomal beta-oxidation are to shorten toxic lipophilic carboxylic acids to facilitate their excretion and to shorten very-long-chain fatty acids prior to mitochondrial beta-oxidation (Mannaerts, G. P. and P. P. Van Veldhoven (1993) Biochimie 75:147-158). Enzymes involved in beta-oxidation include acyl CoA synthetase, carnitine acyltransferase, acyl CoA dehydrogenases, enoyl CoA hydratases, L-3-hydroxyacyl CoA dehydrogenase, β-ketothiolase, 2,4-dienoyl CoA reductase, and isomerase.

Three classes of lipid metabolism enzymes are discussed in further detail. The three classes are lipases, phospholipases and lipoxygenases.

Enoyl-CoA hydratase (EC 4.2.1.17) (ECH) (Minami-Ishii, N. et al. (1989) Eur. J. Biochem. 185:73-78) and 3-2-trans-enoyl-CoA isomerase (EC 5.3.3.8) (ECI) (Mueller-Newen, G. and W. Stoffel (1991) Biol. Chem. Hoppe-Seyler 372:613-624) are two enzymes involved in fatty acid metabolism. ECH catalyzes the hydration of 2-trans-enoyl-CoA into 3-hydroxyacyl-CoA. ECI shifts the 3-double bond of the intermediates of unsaturated fatty acid oxidation to the 2-trans position. Most cells have two fatty-acid beta-oxidation systems, one located in mitochondria and the other in peroxisomes. In mitochondria, ECH and ECI are separate yet structurally related monofunctional enzymes. Peroxisomes contain a trifunctional enzyme (Palosaari, P. M. and J. K. Hiltunen (1990) J. Biol. Chem. 265:2446-2449) consisting of an N-terminal domain that bears both ECH and ECI activity, and a C-terminal domain responsible for 3-hydroxyacyl-CoA dehydrogenase (HCDH) activity.

Lipases

Triglycerides are hydrolyzed to fatty acids and glycerol by lipases. Adipocytes contain lipases that break down stored triacylglycerols, releasing fatty acids for export to other tissues where they are required as fuel. Lipases are widely distributed in animals, plants, and prokaryotes. Triglyceride lipases (ExPASy ENZYME EC 3.1.1.3), also known as triacylglycerol lipases and tributyrases, hydrolyze the ester bond of triglycerides. In higher vertebrates there are at least three tissue-specific isozymes including gastric, hepatic, and pancreatic lipases. These three types of lipases are structurally closely related to each other as well as to lipoprotein lipase. The most conserved region in gastric, hepatic, and pancreatic lipases is centered around a serine residue which is also present in lipases of prokaryotic origin. Mutation in the serine residue renders the enzymes inactive. Gastric, hepatic, and pancreatic lipases hydrolyze lipoprotein triglycerides and phospholipids. Gastric lipases in the intestine aid in the digestion and absorption of dietary fats. Hepatic lipases are bound to and act at the endothelial surfaces of hepatic tissues. Hepatic lipases also play a major role in the regulation of plasma lipids. Pancreatic lipase requires a small protein cofactor, colipase, for efficient dietary lipid hydrolysis. Colipase binds to the C-terminal, non-catalytic domain of lipase, thereby stabilizing an active conformation and considerably increasing the overall hydrophobic binding site. Deficiencies of these enzymes have been identified in man, and all are associated with pathologic levels of circulating lipoprotein particles (Gargouri, Y. et al. (1989) Biochim. Biophys. Acta 1006:255-271; Connelly, P. W. (1999) Clin. Chim. Acta 286:243-255; van Tilbourgh, H. et al. (1999) Biochim Biophys Acta 1441:173-184).

Lipoprotein lipases (ExPASy ENZYME EC 3.1.1.34), also-known as clearing factor lipases, diglyceride lipases, or diacylglycerol lipases, hydrolyze triglycerides and phospholipids present in circulating plasma lipoproteins, including chylomicrons, very low and intermediate density lipoproteins, and high-density lipoproteins (HDL). Together with pancreatic and hepatic lipases, lipoprotein lipases (LPL) share a high degree of primary sequence homology. Both lipoprotein lipases and hepatic lipases are anchored to the capillary endothelium via glycosaminoglycans and can be released by intravenous administration of heparin. LPLs are primarily synthesized by adipocytes, muscle cells, and macrophages. Catalytic activities of LPLs are activated by apolipoprotein C-II and are inhibited by high ionic strength conditions such as 1 M NaCl. LPL deficiencies in humans contribute to metabolic diseases such as hypertriglyceridemia, HDL2 deficiency, and obesity (Jackson, R. L. (1983) in The Enzymes (Boyer, P. D., ed.) Vol. XVI, pp. 141-186, Academic Press, New York N.Y.; Eckel, R. H. (1989) New Engl. J. Med. 320:1060-1068).

Phospholipases

Phospholipases, a group of enzymes that catalyze the hydrolysis of membrane phospholipids, are classified according to the bond cleaved in a phospholipid. They are classified into PLA1, PLA2, PLB, PLC, and PLD families. Phospholipases are involved in many inflammatory reactions by making arachidonate available for eicosanoid biosynthesis. More specifically, arachidonic acid is processed into bioactive lipid mediators of inflammation such as lyso-platelet-activating factor and eicosanoids. The synthesis of arachidonic acid from membrane phospholipids is the rate-limiting step in the biosynthesis of the four major classes of eicosanoids (prostaglandins, prostacyclins, thromboxanes and leukotrienes), which are 20-carbon molecules derived from fatty acids. Eicosanoids are signaling molecules which have roles in pain, fever, and inflammation. The precursor of all eicosanoids is arachidonate, which is generated from phospholipids by phospholipase A₂ and from diacylglycerols by diacylglycerol lipase. Leukotrienes are produced from arachidonate by the action of lipoxygenases (Kaiser, E. et al. (1990) Clin. Biochem. 23:349-370). Furthermore, leukotriene-B4 is known to function in a feedback loop which further increases PLA2 activity (Wijkander, J. et al. (1995) J. Biol. Chem. 270:26543-26549).

The secretory phospholipase A₂ (PLA2) superfamily comprises a number of heterogeneous enzymes whose common feature is to hydrolyze the sn-2 fatty acid acyl ester bond of phosphoglycerides. Hydrolysis of the glycerophospholipids releases free fatty acids and lysophospholipids. PLA2 activity generates precursors for the biosynthesis of biologically active lipids, hydroxy fatty acids, and platelet-activating factor, PLA2s were first described as components of snake venoms, and were later characterized in numerous species. PLA2s have traditionally been classified into several major groups and subgroups based on their amino acid sequences, divalent cation requirements, and location of disulfide bonds. The PLA2s of Groups I, II, and III consist of low molecular weight, secreted, Ca²⁺-dependent proteins. Group IV PLA2s are primarily 85-kDa, Ca²⁺-dependent cytosolic phospholipases. Finally, a number of Ca²⁺-independent PLA2s have been described, which comprise Group V (Davidson, F. F. and E. A. Dennis (1990) J. Mol. Evol. 31:228-238; and Dennis, E. F. (1994) J. Biol Chem. 269:13057-13060).

The first PLA2s to be extensively characterized were the Group I, II, and III PLA2s found in snake and bee venoms. These venom PLA2s share many features with mammalian PLA2s including a common catalytic mechanism, the same Ca²⁺ requirement, and conserved primary and tertiary structures. In addition to their role in the digestion of prey, the venom PLA2s display neurotoxic, myotoxic, anticoagulant, and proinflammatory effects in mammalian tissues. This diversity of pathophysiological effects is due to the presence of specific, high affinity receptors for these enzymes on various cells and tissues (Lambeau, G. et al. (1995) J. Biol. Chem. 270:5534-5540).

PLA2s from Groups I, IIA, IIC, and V have been described in mammalian and avian cells, and were originally characterized by tissue distribution, although the distinction is no longer absolute. Thus, Group I PLA2s were found in the pancreas, Group IIA and IIC were derived from inflammation-associated tissues (e.g., the synovium), and Group V were from cardiac tissue. The pancreatic PLA2s function in the digestion of dietary lipids and have been proposed to play a role in cell proliferation, smooth muscle contraction, and acute lung injury. The Group II inflammatory PLA2s are potent mediators of inflammatory processes and are highly expressed in serum and synovial fluids of patients with inflammatory disorders. These Group II PLA2s are found in most human cell types assayed and are expressed in diverse pathological processes such as septic shock, intestinal cancers, rheumatoid arthritis, and epidermal hyperplasia. A Group V PLA2 has been cloned from brain tissue and is strongly expressed in heart tissue. A human PLA2 was recently cloned from fetal lung, and based on its structural properties, appears to be the first member of a new group of mammalian PLA2s, referred to as Group X. Other PLA2s have been cloned from various human tissues and cell lines, suggesting a large diversity of PLA2s (Chen, J. et al. (1994) J. Biol. Chem. 269:2365-2368; Kennedy, B. P. et al. (1995) J. Biol. Chem. 270: 22378-22385; Komada, M. et al. (1990) Biochem. Biophys. Res. Commun. 168:1059-1065; Cupillard, L. et al. (1997) J. Biol. Chem. 272:15745-15752; and Nalefski, E. A. et al. (1994) J. Biol. Chem. 269:18239-18249).

Phospholipases B (PLB)(ExPASy ENZYME EC 3.1.1.5), also known as lysophospholipase, lecithinase B, or lysolecithinase are widely distributed enzymes that metabolize intracellular lipids, and occur in numerous isoforms. Small isoforms, approximately 15-30 kD, function as hydrolases; large isoforms, those exceeding 60 kD, function both as hydrolases and transacylases. A particular substrate for PLBs, lysophosphatidylcholine, causes lysis of cell membranes when it is formed or imported into a cell. PLBs are regulated by lipid factors including acylcarnitine, arachidonic acid, and phosphatidic acid. These lipid factors are signaling molecules important in numerous pathways, including the inflammatory response (Anderson, R. et al. (1994) Toxicol. Appl. Pharmacol. 125:176-183; Selle, H. et al. (1993); Eur. J. Biochem. 212:411-416).

Phospholipase C (PLC) (ExPASy ENZYME EC 3.1.4.10) plays an important role in transmembrane signal transduction. Many extracellular signaling molecules including hormones, growth factors, neurotransmitters, and immunoglobulins bind to their respective cell surface receptors and activate PLCs. The role of an activated PLC is to catalyze the hydrolysis of phosphatidyl-inositol-4,5-bisphosphate (PIP2), a minor component of the plasma membrane, to produce diacylglycerol and inositol 1,4,5-trisphosphate (IP3). In their respective biochemical pathways, IP3 and diacylglycerol serve as second messengers and trigger a series of intracellular responses. IP3 induces the release of Ca²⁺ from internal cellular storage, and diacylglycerol activates protein kinase C (PKC). Both pathways are part of transmembrane signal transduction mechanisms which regulate cellular processes which include secretion, neural activity, metabolism, and proliferation.

Several distinct isoforms of PLC have been identified and are categorized as PLC-beta, PLC-gamma, and PLC-delta. Subtypes are designated by adding Arabic numbers after the Greek letters, eg. PLC-β-1. PLCs have a molecular mass of 62-68 kDa, and their amino acid sequences show two regions of significant similarity. The first region, designated X, has about 170 amino acids, and the second, or Y region, contains about 260 amino acids.

The catalytic activities of the three isoforms of PLC are dependent upon Ca²⁺. It has been suggested that the binding sites for Ca²⁺ in the PLCs are located in the Y-region, one of two conserved regions. The hydrolysis of common inositol-containing phospholipids, such as phosphatidylinositol (PI), phosphatidylinositol 4-monophosphate (PIP), and phosphatidylinositol 4,5-bisphosphate (PIP2), by any of the isoforms yields cyclic and noncyclic inositol phosphates (Rhee, S. G. and Y. S. Bae (1997) J. Biol. Chem. 272:15045-15048).

All mammalian PLCs contain a pleckstrin homology (PH) domain which is about 100 amino acids in length and is composed of two antiparallel beta sheets flanked by an amphipathic alpha helix. PH domains target PLCs to the membrane surface by interacting with either the beta/gamma subunits of G proteins or PIP2 (PROSITE PDOC50003).

Phospholipase D (PLD) (ExPASy ENZYME EC 3.1.4.4), also known as lecithinase D, lipophosphodiesterase II, and choline phosphatase catalyzes the hydrolysis of phosphatidylcholine and other phospholipids to generate phosphatidic acid. PLD plays an important role in membrane vesicle trafficking, cytoskeletal dynamics, and transmembrane signal transduction. In addition, the activation of PLD is involved in cell differentiation and growth (reviewed in Liscovitch, M. (2000) Biochem. J. 345:401-415).

PLD is activated in mammalian cells in response to diverse stimuli that include hormones, neurotransmitters, growth factors, cytokines, activators of protein kinase C, and agonist binding to G-protein-coupled receptors. At least two forms of mammalian PLD, PLD1 and PLD2, have been identified. PLD1 is activated by protein kinase C alpha and by the small GTPases ARF and RhoA. (Houle, M. G. and S. Bourgoin (1999) Biochim. Biophys. Acta 1439:135-149). PLD2 can be selectively activated by unsaturated fatty acids such as oleate (Kim, J. H. (1999) FEBS Lett. 454:42-46).

Lipoxygenases

Lipoxygenases (ExPASy ENZYME EC 1.13.11.12) are non-heme iron-containing enzymes that catalyze the dioxygenation of certain polyunsaturated fatty acids such as lipoproteins. Lipoxygenases are found widely in plants, fungi, and animals. Several different lipoxygenase enzymes are known, each having a characteristic oxidation action. In animals, there are specific lipoxygenases that catalyze the dioxygenation of arachidonic acid at the carbon-3, 5, 8, 11, 12, and 15 positions. These enzymes are named after the position of arachidonic acid that they dioxygenate. Lipoxygenases have a single polypeptide chain with a molecular mass of ˜75-80 kDa in animals. The proteins have an N-terminal-barrel domain and a larger catalytic domain containing a single atom of non-heme iron. Oxidation of the ferric enzyme to an active form is required for catalysis (Yamamoto, S. (1992) Biochim. Biophys. Acta 1128:117-131; Brash, A. R. (1999) J. Biol. Chem. 274:23679-23682). A variety of lipoxygenase inhibitors exist and are classified into five major categories according to their mechanism of inhibition. These include antioxidants, iron chelators, substrate analogues, lipoxygenase-activating protein inhibitors, and, finally, epidermal growth factor-receptor inhibitors.

3-Lipoxygenase, also known as e-LOX-3 or Aloxe3 has recently been cloned from murine epidermis. Aloxe3 resides on mouse chromosome 11, and the deduced amino acid sequence for Aloxe3 is 54% identical to the 12-lipoxygenase sequences (Kinzig, A. (1999) Genomics 58:158-164).

5-Lipoxygenase (5-LOX, ExPASy ENZYME EC 1.13.11.34), also known as arachidonate:oxygen 5-oxidoreductase, is found primarily in white blood cells, macrophages, and mast cells. 5-LOX converts arachidonic acid first to 5-hydroperoxyeicosatetraenoic acid (5-HPETE) and then to leukotriene (LTA4 (5,6-oxido-7,9,11,14-eicosatetraenoic acid)). Subsequent conversion of leukotriene A4 by leukotriene A4 hydrolase yields the potent neutrophil chemoattractant leukotriene B4. Alternatively, conjugation of LTA4 with glutathione by leukotriene C4 synthase plus downstream metabolism leads to the cysteinyl leukotrienes that influence airway reactivity and mucus secretion, especially in asthmatics. Most lipoxygenases require no other cofactors or proteins for activity. In contrast, the mammalian 5-LOX requires calcium and ATP, and is activated in the presence of a 5-LOX activating protein (LAP). FLAP itself binds to arachidonic acid and supplies 5-LOX with substrate (Lewis, R. A. et al. (1990) New Engl. J. Med. 323:645-655). The expression levels of 5-LOX and FLAP are found to be increased in the lungs of patients with plexogenic (primary) pulmonary hypertension (Wright, L. et al. (1998) Am. J. Respir. Crit. Care Med. 157:219-229).

12-Lipoxygenase (12-LOX, ExPASy ENZYME: EC 1.13.11.31) oxygenates arachidonic acid to form 12-hydroperoxyeicosatetraenoic acid (12-HPETE). Mammalian 12-lipoxygenases are named after the prototypical tissues of their occurrence (hence, the leukocyte, platelet, or epidermal types). Platelet-type 12-LOX has been found to be the predominant isoform in epidermal skin specimens and epidermoid cells. Leukocyte 12-LOX was first characterized extensively from porcine leukocytes and was found to have a rather broad distribution in mammalian tissues by immunochemical assays. Besides tissue distribution, the leukocyte 12-LOX is distinguished from the platelet-type enzyme by its ability to form 15-HPETE, in addition to 12-HPETE, from arachidonic acid substrate. Leukocyte 12-LOX is highly related to 15-lipoxgenase (15-LOX) in that both are dual specificity lipoxygenases, and they are about 85% identical in primary structure in higher mammals. Leukocyte 12-LOX is found in tracheal epithelium, leukocytes, and macrophages (Conrad, D. J. (1999) Clin. Rev. Allergy Immunol. 17:71-89).

15-Lipoxygenase (15-LOX; ExPASy ENZYME: EC 1.13.11.33) is found in human reticulocytes, airway epithelium, and eosinophils. 15-LOX has been detected in atherosclerotic lesions in mammals, specifically rabbit and man. The enzyme, in addition to its role in oxidative modification of lipoproteins, is important in the inflammatory reaction in atherosclerotic lesions. 15-LOX has been shown to be induced in human monocytes by the cytokine IL-4, which is known to be implicated in the inflammatory process (Kuhn, H. and S. Borngraber (1999) Adv. Exp. Med. Biol. 447:5-28).

A variety of lipolytic enzymes with a GDSL-like motif as part of the active site have been identified. Members of this family include a lipase/acylhydrolase, thermolabile hemolysin and rabbit phospholipase (AdRab-B)(Interpro entry IPR001087, http://www.sanger.acuk). A homolog of AdRab-B is guinea pig intestinal phospholipase B, a calcium-independent phospholipase that contributes to lipid digestion as an ectoenzyme by sequentially hydrolyzing the acyl ester bonds of glycerophospholipids. Phospholipase B also has a role in male reproduction (Delagebeaudeuf, C. et al. (1998) J. Biol. Chem. 273:13407-13414).

Lipid-Associated Molecules and Disease

Lipids and their associated proteins have roles in human diseases and disorders. Increased synthesis of long-chain fatty acids occurs in neoplasms including those of the breast, prostate, ovary, colon and endometrium.

In the arterial disease atherosclerosis, fatty lesions form on the inside of the arterial wall. These lesions promote the loss of arterial flexibility and the formation of blood clots (Guyton, supra). There is a strong inverse correlation between the levels of plasma HDL and risk of premature coronary heart disease. Absence of the LDL receptor, the cause of familial hypercholesterolemia, leads to increased plasma cholesterol levels and ultimately to atherosclerosis (Stryer, supra, pp. 691-702). Oxysterols are present in human atherosclerotic plaques and are believed to play an active role in plaque development (Brown, A. J. (1999) Atherosclerosis 142:1-28). Lipases, phospholipases, and lipoxygenases are thought to contribute to complex diseases, such as atherosclerosis, obesity, arthritis, asthma, and cancer, as well as to single gene defects, such as Wolman's disease and Type I hyperlipoproteinemia.

Steatosis, or fatty liver, is characterized by the accumulation of triglycerides in the liver and may occur in association with a variety of conditions including alcoholism, diabetes, obesity, and prolonged parenteral nutrition. Steatosis may lead to fibrosis and cirrhosis of the liver.

Niemann-Pick diseases types A and B are caused by accumulation of sphingomyelin (a sphingolipid) and other lipids in the central nervous system due to a defect in the enzyme sphingomyelinase, leading to neurodegeneration and lung disease. Niemann-Pick disease type C results from a defect in cholesterol transport, leading to the accumulation of sphingomyelin and cholesterol in lysosomes and a secondary reduction in sphingomyelinase activity. Neurological symptoms such as grand mal seizures, ataxia, and loss of previously learned speech, manifest 1-2 years after birth. A mutation in the NPC protein, which contains a putative cholesterol-sensing domain, was found in a mouse model of Niemann-Pick disease type C (Fauci, (1994) Harrison's Principles of Internal Medicine, McGraw-Hill, New York N.Y., p. 2175; Loftus, S. K. et al. (1997) Science 277:232-235).

Tay-Sachs disease is an autosomal recessive, progressive neurodegenerative disorder caused by the accumulation of the G_{M2} ganglioside in the brain (Igdoura, S. A. et al. (1999) Hum. Mol. Genet. 8:1111-1116) due to a deficiency of the enzyme hexosaminidase A. The disease is characterized by the onset of developmental retardation, followed by paralysis, dementia, blindness, and usually death within the second or third year of life. Confirmatory evidence of Tay-Sachs disease is obtained at autopsy upon the identification of ballooned neurons in the central nervous system (OMIM #272800). In the case of Tay-Sachs disease, cortical pyramidal neurons undergo a second round of dendritogenesis (Walkley, S. U. et al. (1998) Ann. N.Y. Acad. Sci. 845:188-99).

Other diseases are also associated with defects in sialidase activity. G_{M1} gangliosidosis and Morquio B disease both arise from beta-galactosidase deficiency, although the diseases present with distinct phenotypes. Sialidosis arises from a neuraminidase deficiency but presents with symptoms similar to gangliosidosis. A likely reason for the overlapping phenotypes of sialidase deficiencies is the presence of these enzymes in a complex in lysosomes (Callahan, J. W. (1999) Biochim. Biophys. Acta. 1455:85-103).

PLAs are implicated in a variety of disease processes. For example, PLAs are found in the pancreas, in cardiac tissue, and in inflammation-associated tissues. Pancreatic PLAs function in the digestion of dietary lipids and have been proposed to play a role in cell proliferation, smooth muscle contraction, and acute lung injury. Inflammatory PLAs are potent mediators of inflammatory processes and are highly expressed in serum and synovial fluids of patients with inflammatory disorders. Additionally, inflammatory PLAs are found in most human cell types and are expressed in diverse pathological processes such as septic shock, intestinal cancers, rheumatoid arthritis, and epidermal hyperplasia.

The role of PLBs in human tissues has been investigated in various research studies. Hydrolysis of lysophosphatidylcholine by PLBs causes lysis in erythrocyte membranes (Selle et al., supra). Similarly, Endresen, M. J. et al. (1993; Scand. J. Clin. Invest. 53:733-739) reported that the increased hydrolysis of lysophosphatidylcholine by PLB in pre-eclamptic women causes release of free fatty acids into the sera. In renal studies, PLB was shown to protect Na⁺,K⁺-ATPase from the cytotoxic and cytolytic effects of cyclosporin A (Anderson et al., supra).

Lipases, phospholipases, and lipoxygenases are thought to contribute to complex diseases, such as atherosclerosis, obesity, arthritis, asthma, and cancer, as well as to single gene defects, such as Wolman's disease and Type I hyperlipoproteinemia.

Expression Profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry.

One area in particular in which microarrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

Colon Cancer

While soft tissue sarcomas are relatively rare, more than 50% of new patients diagnosed with the disease will die from it. The molecular pathways leading to the development of sarcomas are relatively unknown, due to the rarity of the disease and variation in pathology. Colon cancer evolves through a multi-step process whereby pre-malignant colonocytes undergo a relatively defined sequence of events leading to tumor formation. Several factors participate in the process of tumor progression and malignant transformation including genetic factors, mutations, and selection.

To understand the nature of gene alterations in colorectal cancer, a number of studies have focused on the inherited syndromes. Familial adenomatous polyposis SAP), is caused by mutations in the adenomatous polyposis coli gene (APC), resulting in truncated or inactive forms of the protein. This tumor suppressor gene has been mapped to chromosome Sq. Hereditary nonpolyposis colorectal cancer (HNPCC) is caused by mutations in mis-match repair genes. Although hereditary colon cancer syndromes occur in a small percentage of the population and most colorectal cancers are considered sporadic, knowledge from studies of the hereditary syndromes can be generally applied. For instance, somatic mutations in APC occur in at least 80% of sporadic colon tumors. APC mutations are thought to be the initiating event in the disease. Other mutations occur subsequently.

Approximately 50% of colorectal cancers contain activating mutations in ras, while 85% contain inactivating mutations in p53. Changes in all of these genes lead to gene expression changes in colon cancer.

Lung Cancer

The potential application of gene expression profiling is particularly relevant to improving diagnosis, prognosis, and treatment of cancer, such as lung cancer. Lung cancer is the leading cause of cancer death in the United States, affecting more than 100,000 men and 50,000 women each year.

Nearly 90% of the patients diagnosed with lung cancer are cigarette smokers. Tobacco smoke contains thousands of noxious substances that induce carcinogen metabolizing enzymes and covalent DNA adduct formation in the exposed bronchial epithelium. In nearly 80% of patients diagnosed with lung cancer, metastasis has already occurred. Most commonly lung cancers metastasize to pleura, brain, bone, pericardium, and liver. The decision to treat with surgery, radiation therapy, or chemotherapy is made on the basis of tumor histology, response to growth factors or hormones, and sensitivity to inhibitors or drugs. With current treatments, most patients die within one year of diagnosis. Earlier diagnosis and a systematic approach to identification, staging, and treatment of lung cancer could positively affect patient outcome.

Lung cancers progress through a series of morphologically distinct stages from hyperplasia to invasive carcinoma. Malignant lung cancers are divided into two groups comprising four histopathological classes. The Non Small Cell Lung Carcinoma (NSCLC) group includes squamous cell carcinomas, adenocarcinomas, and large cell carcinomas and accounts for about 70% of all lung cancer cases. Adenocarcinomas typically arise in the peripheral airways and often form mucin secreting glands. Squamous cell carcinomas typically arise in proximal airways. The histogenesis of squamous cell carcinomas may be related to chronic inflammation and injury to the bronchial epithelium, leading to squamous metaplasia. The Small Cell Lung Carcinoma (SCLC) group accounts for about 20% of lung cancer cases. SCLCs typically arise in proximal airways and exhibit a number of paraneoplastic syndromes including inappropriate production of adrenocorticotropin and anti-diuretic hormone.

Lung cancer cells accumulate numerous genetic lesions, many of which are associated with cytologically visible chromosomal aberrations. The high frequency of chromosomal deletions associated with lung cancer may reflect the role of multiple tumor suppressor loci in the etiology of this disease. Deletion of the short arm of chromosome 3 is found in over 90% of cases and represents one of the earliest genetic lesions leading to lung cancer. Deletions at chromosome arms 9p and 17p are also common. Other frequently observed genetic lesions include overexpression of telomerase, activation of oncogenes such as K-ras and c-myc, and inactivation of tumor suppressor genes such as RB, p53 and CDKN2.

Genes differentially regulated in lung cancer have been identified by a variety of methods. Using mRNA differential display technology, Manda et al. (1999; Genomics 51:5-14) identified five genes differentially expressed in lung cancer cell lines compared to normal bronchial epithelial cells. Among the known genes, pulmonary surfactant apoprotein A and alpha 2 macroglobulin were down regulated whereas nm23H1 was upregulated. Petersen et al. (2000; Int J. Cancer, 86:512-517) used suppression subtractive hybridization to identify 552 clones differentially expressed in lung tumor derived cell lines, 205 of which represented known genes. Among the known genes, thrombospondin-1, fibronectin, intercellular adhesion molecule 1, and cytokeratins 6 and 18 were previously observed to be differentially expressed in lung cancers. Wang et al. (2000; Oncogene 19:1519-1528) used a combination of microarray analysis and subtractive hybridization to identify 17 genes differentially overexpresssed in squamous cell carcinoma compared with normal lung epithelium. Among the known genes they identified were keratin isoform 6, KOC, SPRC, IGFb2, connexin 26, plakofillin 1 and cytokeratin 13.

Ovarian Cancer

Ovarian cancer is the leading cause of death from a gynecologic cancer. The majority of ovarian cancers are derived from epithelial cells, and 70% of patients with epithelial ovarian cancers present with late-stage disease. As a result, the long-term survival rate for this disease is very low. Identification of early-stage markers for ovarian cancer would significantly increase the survival rate. Genetic variations involved in ovarian cancer development include mutation of p53 and microsatellite instability. Gene expression patterns likely vary when normal ovary is compared to ovarian tumors.

More than 180,000 new cases of breast cancer are diagnosed each year, and the mortality rate for breast cancer approaches 10% of all deaths in females between the ages of 45-54 (Gish, K. (1999) AWIS Magazine 28:7-10). However the survival rate based on early diagnosis of localized breast cancer is extremely high (97%), compared with the advanced stage of the disease in which the tumor has spread beyond the breast (22%). Current procedures for clinical breast examination are lacking in sensitivity and specificity, and efforts are underway to develop comprehensive gene expression profiles for breast cancer that may be used in conjunction with conventional screening methods to improve diagnosis and prognosis of this disease (Perou, C. M. et al. (2000) Nature 406:747-752).

Breast Cancer

Mutations in two genes, BRCA1 and BRCA2, are known to greatly predispose a woman to breast cancer and may be passed on from parents to children (Gish, supra). However, this type of hereditary breast cancer accounts for only about 5% to 9% of breast cancers, while the vast majority of breast cancer is due to non-inherited mutations that occur in breast epithelial cells.

The relationship between expression of epidermal growth factor (EGF) and its receptor, EGFR, to human mammary carcinoma has been particularly well studied. (See Khazaie, K. et al. (1993) Cancer and Metastasis Rev. 12:255-274, and references cited therein for a review of this area.) Overexpression of EGFR, particularly coupled with down-regulation of the estrogen receptor, is a marker of poor prognosis in breast cancer patients. In addition, EGFR expression in breast tumor metastases is frequently elevated relative to the primary tumor, suggesting that EGFR is involved in tumor progression and metastasis. This is supported by accumulating evidence that EGF has effects on cell functions related to metastatic potential, such as cell motility, chemotaxis, secretion and differentiation. Changes in expression of other members of the erbB receptor family, of which EGFR is one, have also been implicated in breast cancer. The abundance of erbB receptors, such as HER-2/neu, HER-3, and HER-4, and their ligands in breast cancer points to their functional importance in the pathogenesis of the disease, and may therefore provide targets for therapy of the disease (Bacus, S. S. et al. (1994) Am. J. Clin. Pathol. 102:S13-S24). Other known markers of breast cancer include a human secreted frizzled protein mRNA that is downregulated in breast tumors; the matrix G1a protein which is overexpressed in human breast carcinoma cells; Drg1 or RTP, a gene whose expression is diminished in colon, breast, and prostate tumors; maspin, a tumor suppressor gene downregulated in: invasive breast carcinomas; and CaN19, a member of the S100 protein family, all of which are down-regulated in mammary carcinoma cells relative to normal mammary epithelial cells (Zhou, Z. et al. (1998) Int. J. Cancer 78:95-99; Chen, L. et al. (1990) Oncogene 5:1391-1395; Ulrix, W. et al (1999) FEBS Lett 455:23-26; Sager, R. et al. (1996) Curr. Top. Microbiol. Immunol. 213:51-64; and Lee, S. W. et al. (1992) Proc. Natl. Acad. Sci. USA 89:2504-2508).

Cell lines derived from human mammary epithelial cells at various stages of breast cancer provide a useful model to study the process of malignant transformation and tumor progression as it has been shown that these cell lines retain many of the properties of their parental tumors for lengthy culture periods (Wistuba, I. I. et al. (1998) Clin. Cancer Res. 4:2931-2938). Such a model is particularly useful for comparing phenotypic and molecular characteristics of human mammary epithelial cells at various stages of malignant transformation.

Vascular Biology

Human aortic endothelial cells (HMVECdNeos) are primary cells derived from the endothelium of the microvasculature of human skin. HMVECdNeos have been used as an experimental model for investigating in vitro the role of the endothelium in human vascular biology. Activation of the vascular endothelium is considered a central event in a wide range of both physiological and pathophysiological processes, such as vascular tone regulation, coagulation and thrombosis, atherosclerosis, and inflammation.

Human umbilical vein endothelial cells (HUVECs) are a primary cell line derived from the endothelium of the human umbilical vein. HUVECs have been used extensively to study the functional biology of human endothelial cells in vitro. Activation of vascular endothelium is considered a central event in a wide range of both physiological and pathophysiological processes, such as vascular tone regulation, coagulation and thrombosis, atherosclerosis, and inflammation.

Tumor necrosis factor-alpha (TNF-α) [94948-59-1] is a pleiotropic cytokine that plays a central role in mediation of the inflammatory response through activation of multiple signal transduction pathways. TNF-α is produced by activated lymphocytes, macrophages, and other white blood-cells, and is known to activate endothelial cells. Monitoring the endothelial cell response to TNF-α at the level of mRNA expression can provide information necessary for better understanding of both TNF-α signaling and endothelial cell biology.

Immunological Disorders

Human peripheral blood mononuclear cells (PBMCs) represent the major cellular components of the immune system. PBMCs contain about 12% B lymphocytes, 25% CD4+ and 15% CD8+ lymphocytes, 20% NK cells, 25% monocytes, and 3% various cells that include dendritic cells and progenitor cells. The proportions, as well as the biology of these cellular components tend to vary slightly between healthy individuals, depending on factors such as age, gender, past. medical history, and genetic background.

Staphylococcal exotoxins such as staphlococcal exotoxin B (SEB) specifically activate human T cells, expressing an appropriate TCR-Vbeta chain. Although polyclonal in nature, T cells activated by Staphylococcal exotoxins require antigen presenting cells (APCs) to present the exotoxin molecules to the T cells and deliver the costimulatory signals required for optimum T cell activation. Although Staphylococcal exotoxins must be presented to T cells by APCs, these molecules are not required to be processed by APC. Indeed, Staphylococcal exotoxins directly bind to a non-polymorphic portion of the human MHC class II molecules, bypassing the need for capture, cleavage, and binding of the peptides to the polymorphic antigenic groove of the MHC class II molecules.

Prostate Cancer

Prostate cancer is a common malignancy in men over the age of 50, and the incidence increases with age. In the US, there are approximately 132,000 newly diagnosed cases of prostate cancer and more than 33,000 deaths from the disorder each year.

Once cancer cells arise in the prostate, they are stimulated by testosterone to a more rapid growth. Thus, removal of the testes can indirectly reduce both rapid growth and metastasis of the cancer. Over 95 percent of prostatic cancers are adenocarcinomas which originate in the prostatic acini. The remaining 5 percent are divided between squamous cell and transitional cell carcinomas, both of which arise in the prostatic ducts or other parts of the prostate gland.

As with most tumors, prostate cancer develops through a multistage progression ultimately resulting in an aggressive tumor phenotype. The initial step in tumor progression involves the hyperproliferation of normal luminal and/or basal epithelial cells. Androgen responsive cells become hyperplastic and evolve into early-stage tumors. Although early-stage tumors are often androgen sensitive and respond to androgen ablation, a population of androgen independent cells evolve from the hyperplastic population. These cells represent a more advanced form of prostate tumor that may become invasive and potentially become metastatic to the bone, brain, or lung. A variety of genes may be differentially expressed during tumor progression. For example, loss of heterozygosity (LOH) is frequently observed on chromosome 8p in prostate cancer. Fluorescence in situ hybridization (FISH) revealed a deletion for at least 1 locus on 8p in 29 (69%) tumors, with a significantly higher frequency of the deletion on 8p21.2-p21.1 in advanced prostate cancer than in localized prostate cancer, implying that deletions on 8p22-p21.3 play an important role in tumor differentiation, while 8p21.2-p21.1 deletion plays a role in progression of prostate cancer (Oba, K. et al. (2001) Cancer Genet. Cytogenet. 124: 20-26).

A primary diagnostic marker for prostate cancer is prostate specific antigen (PSA). PSA is a tissue-specific serine protease almost exclusively produced by prostatic epithelial cells. The quantity of PSA correlates with the number and volume of the prostatic epithelial cells, and consequently, the levels of PSA are an excellent indicator of abnormal prostate growth. Men with prostate cancer exhibit an early linear increase in PSA levels followed by an exponential increase prior to diagnosis. However, since PSA levels are also influenced by factors such as inflammation, androgen and other growth factors, some scientists maintain that changes in PSA levels are not useful in detecting individual cases of prostate cancer.

Current areas of cancer research provide additional prospects for markers as well as potential therapeutic targets for prostate cancer. Several growth factors have been shown to play a critical role in tumor development, growth, and progression. The growth factors Epidermal Growth Factor (EGF), Fibroblast Growth Factor (FGF), and Tumor Growth Factor alpha (TGFx) are important in the growth of normal as well as hyperproliferative prostate epithelial cells, particularly at early stages of tumor development and progression, and affect signaling pathways in these cells in various ways (Lin, J. et al. (1999) Cancer Res. 59:2891-2897; Putz, T. et al. (1999) Cancer Res. 59:227-233). The TGF-β family of growth factors are generally expressed at increased levels in human cancers and the high expression levels in many cases correlates with advanced stages of malignancy and poor survival (Gold, L. I. (1999) Crit. Rev. Oncog. 10:303-360). Finally, there are human cell lines representing both the androgen-dependent stage of prostate cancer (LNCap) as well as the androgen-independent, hormone refractory stage of the disease (PC3 and DU-145) that have proved useful in studying gene expression patterns associated with the progression of prostate cancer, and the effects of cell treatments on these expressed genes (Chung, T. D. (1999) Prostate 15:199-207).

There is a need in the art for new compositions, including nucleic acids and proteins, for the diagnosis, prevention, and treatment of cancer, cardiovascular, neurological, autoimmune/inflammatory, and gastrointestinal disorders, and disorders of lipid metabolism.

### SUMMARY OF THE INVENTION

Various embodiments of the invention provide purified polypeptides, lipid-associated molecules, referred to collectively as ‘LIPAM’ and individually as ‘LIPAM-1,’ ‘LIPAM-2,’ ‘LIPAM-3,’ ‘LIPAM-4,’ ‘LIPAM-5,’ ‘LIPAM-6,’ ‘LIPAM-7,’ ‘LIPAM-8,’ ‘LIPAM-9,’ ‘LIPAM-10,’ ‘LIPAM-1,’ ‘LIPAM-12,’ ‘LIPAM-13,’ ‘LIPAM-14,’ ‘LIPAM-15,’ ‘LIPAM-16,’ ‘LIPAM-17,’ ‘LIPAM-18,’ ‘LIPAM-19,’ ‘LIPAM-20,’ and ‘LIPAM-21’ and methods for using these proteins and their encoding polynucleotides for the detection, diagnosis, and treatment of diseases and medical conditions. Embodiments also provide methods for utilizing the purified lipid-associated molecules and/or their encoding polynucleotides for facilitating the drug discovery process, including determination of efficacy, dosage, toxicity, and pharmacology. Related embodiments provide methods for utilizing the purified lipid-associated molecules and/or their encoding polynucleotides for investigating the pathogenesis of diseases and medical conditions.

An embodiment provides an isolated polypeptide selected from the group consisting of a) a polypeptide-comprising an amino acid sequence selected from the group consisting of SEQ ID NO:1-21, b) a polypeptide comprising a naturally occurring amino acid sequence at least 90% identical or at least about 90% identical to an amino acid sequence selected from the group consisting of SEQ ID NO:1-21, c) a biologically active fragment of a polypeptide having an amino acid sequence selected from the group consisting of SEQ ID NO:1-21, and d) an immunogenic fragment of a polypeptide having an amino acid sequence selected from the group consisting of SEQ ID NO:1-21. Another embodiment provides an isolated polypeptide comprising an amino acid sequence of SEQ ID NO:1-21.

Still another embodiment provides an isolated polynucleotide encoding a polypeptide selected from the group consisting of a) a polypeptide comprising an amino acid sequence selected from the group consisting of SEQ ID NO:1-21, b) a polypeptide comprising a naturally occurring amino acid sequence at least 90% identical or at least about 90% identical to an amino acid sequence selected from the group consisting of SEQ ID NO:1-21, c) a biologically active fragment of a polypeptide having an amino acid sequence selected from the group consisting of SEQ ID NO:1-21, and d) an immunogenic fragment of a polypeptide having an amino acid sequence selected from the group consisting of SEQ ID NO:1-21. In another embodiment, the polynucleotide encodes a polypeptide selected from the group consisting of SEQ ID NO:1-21. In an alternative embodiment, the polynucleotide is selected from the group consisting of SEQ ID NO:22-42.

Still another embodiment provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-21, b) a polypeptide comprising a naturally occurring amino acid sequence at least 90% identical or at least about 90% identical to an amino acid sequence selected from the group consisting of SEQ ID NO:1-21, c) a biologically active fragment of a polypeptide having an amino acid sequence selected from the group consisting of SEQ ID NO:1-21, and d) an immunogenic fragment of a polypeptide having an amino acid sequence selected from the group consisting of SEQ ID NO:1-21. Another embodiment provides a cell transformed with the recombinant polynucleotide. Yet another embodiment provides a transgenic organism comprising the recombinant polynucleotide.

Another embodiment provides a method for producing a polypeptide selected from the group consisting of a) a polypeptide comprising an amino acid sequence selected from the group consisting of SEQ ID NO:1-21, b) a polypeptide comprising a naturally occurring amino acid sequence at least 90% identical or at least about 90% identical to an amino acid sequence selected from the group consisting of SEQ ID NO:1-21, c) a biologically active fragment of a polypeptide having an amino acid sequence selected from the group consisting of SEQ ID NO:1-21, and d) an immunogenic fragment of a polypeptide having an amino acid sequence selected from the group consisting of SEQ ID NO:1-21. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence selected from the group consisting of SEQ ID NO:1-21, b) a polypeptide comprising a naturally occurring amino acid sequence at least 90% identical or at least about 90% identical to an amino acid sequence selected from the group consisting of SEQ ID NO:1-21, c) a biologically active fragment of a polypeptide having an amino acid sequence selected from the group consisting of SEQ ID NO:1-21, and d) an immunogenic fragment of a polypeptide having an amino acid sequence selected from the group consisting of SEQ ED NO:1-21.

Still yet another embodiment provides an isolated polynucleotide selected from the group consisting of a) a polynucleotide comprising a polynucleotide sequence selected from the group consisting of SEQ ID NO:22-42, b) a polynucleotide comprising a naturally occurring polynucleotide sequence at least 90% identical or at least about 90% identical to a polynucleotide sequence selected from the group consisting of SEQ ID NO:22-42, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides.

Yet another embodiment provides a method for detecting a target polynucleotide in a sample, said target polynucleotide being selected from the group consisting of a) a polynucleotide comprising a polynucleotide sequence selected from the group consisting of SEQ ID NO:22-42, b) a polynucleotide comprising a naturally occurring polynucleotide sequence at least 90% identical or at least about 90% identical to a polynucleotide sequence selected from the group consisting of SEQ ID NO:22-42, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other embodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence selected from the group consisting of SEQ ID NO:22-42, b) a polynucleotide comprising a naturally occurring polynucleotide sequence at least 90% identical or at least about 90% identical to a polynucleotide sequence selected from the group consisting of SEQ ID NO:22-42, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof. In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence selected from the group consisting of SEQ ID NO:1-21, b) a polypeptide comprising a naturally occurring amino acid sequence at least 90% identical or at least about 90% identical to an amino acid sequence selected from the group consisting of SEQ ID NO:1-21, c) a biologically active fragment of a polypeptide having an amino acid sequence selected from the group consisting of SEQ ID NO:1-21, and d) an immunogenic fragment of a polypeptide having an amino acid sequence selected from the group consisting of SEQ ID NO:1-21, and a pharmaceutically acceptable excipient. In one embodiment, the composition can comprise an amino acid sequence selected from the group consisting of SEQ ID NO:1-21. Other embodiments provide a method of treating a disease or condition associated with decreased or abnormal expression of functional LIPAM, comprising administering to a patient in need of such treatment the composition.

Yet another embodiment provides a method for screening a compound for effectiveness as an agonist of a polypeptide selected from the group consisting of a) a polypeptide comprising an amino acid sequence selected from the group consisting of SEQ ID NO:1-21, b) a polypeptide comprising a naturally occurring amino acid sequence at least 90% identical or at least about 90% identical to an amino acid sequence selected from the group consisting of SEQ ID NO:1-21, c) a biologically active fragment of a polypeptide having an amino acid sequence selected from the group consisting of SEQ ID NO:1-21, and d) an immunogenic fragment of a polypeptide having an amino acid sequence selected from the group consisting of SEQ ID NO:1-21. The method comprises a) contacting a sample comprising the polypeptide with a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional LIPAM, comprising administering to a patient in need of such treatment the composition.

Still yet another embodiment provides a method for screening a compound for effectiveness as an antagonist of a polypeptide selected from the group consisting of a) a polypeptide comprising an amino acid sequence selected from the group consisting of SEQ ID NO:1-21, b) a polypeptide comprising a naturally occurring amino acid sequence at least 90% identical or at least about 90% identical to an amino acid sequence selected from the group consisting of SEQ ID NO:1-21, c) a biologically active fragment of a polypeptide having an amino acid sequence selected from the group consisting of SEQ ID NO:1-21, and d) an immunogenic fragment of a polypeptide having an amino acid sequence selected from the group consisting of SEQ ID NO:1-21. The method comprises a) contacting a sample comprising the polypeptide with a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional LIPAM, comprising administering to a patient in need of such treatment the composition.

Another embodiment provides a method of screening for a compound that specifically binds to a polypeptide selected from the group consisting of a) a polypeptide comprising an amino acid sequence selected from the group consisting of SEQ ID NO:1-21, b) a polypeptide comprising a naturally occurring amino acid sequence at least 90% identical or at least about 90% identical to an amino acid sequence selected from the group consisting of SEQ ID NO:1-21, c) a biologically active fragment of a polypeptide having an amino acid sequence selected from the group consisting of SEQ ID NO:1-21, and d) an immunogenic fragment of a polypeptide having an amino acid sequence selected from the group consisting of SEQ ID NO:1-21. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence selected from the group consisting of SEQ ID NO:1-21, b) a polypeptide comprising a naturally occurring amino acid sequence at least 90% identical or at least about 90% identical to an amino acid sequence selected from the group consisting of SEQ ID NO:1-21, c) a biologically active fragment of a polypeptide having an amino acid sequence selected from the group consisting of SEQ ID NO:1-21, and d) an immunogenic fragment of a polypeptide having an amino acid sequence selected from the group consisting of SEQ ID NO:1-21. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence selected from the group consisting of SEQ ID NO:22-42, the method comprising a) contacting a sample comprising the target polynucleotide with a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:22-42, ii) a polynucleotide comprising a naturally occurring polynucleotide sequence at least 90% identical or at least about 90% identical to a polynucleotide sequence selected from the group consisting of SEQ ID NO:22-42, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:22-42, ii) a polynucleotide comprising a naturally occurring polynucleotide sequence at least 90% identical or at least about 90% identical to a polynucleotide sequence selected from the group consisting of SEQ ID NO:22-42, iii) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog, and the PROTEOME database identification numbers and annotations of PROTEOME database homologs, for polypeptide embodiments of the invention. The probability scores for the matches between each polypeptide and its homolog(s) are also shown.

Table 3 shows structural features of polypeptide embodiments, including predicted motifs and domains, along with the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

Table 8 shows single nucleotide polymorphisms found in polynucleotide sequences of the invention, along with allele frequencies in different human populations.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

### DEFINITIONS

“LIPAM” refers to the amino acid sequences of substantially purified LIPAM obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of LIPAM. Agonists may include proteins, nucleic acids, carbohydrates, small molecules, or any other compound or composition which modulates the activity of LIPAM either by directly interacting with LIPAM or by acting on components of the biological pathway in which LIPAM participates.

An “allelic variant” is an alternative form of the gene encoding LIPAM. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding LIPAM include those sequences with deletions, insertions, or substitutions of different nucleotides, resulting in a polypeptide the same as LIPAM or a polypeptide with at least one functional characteristic of LIPAM. Included within this definition are polymorphisms which may or may not be readily detectable using a particular oligonucleotide probe of the polynucleotide encoding LIPAM, and improper or unexpected hybridization to allelic variants, with a locus other than the normal chromosomal locus for the polynucleotide encoding LIPAM. The encoded protein may also be “altered,” and may contain deletions, insertions, or substitutions of amino acid residues which produce a silent change and result in a functionally equivalent LIPAM. Deliberate amino acid substitutions may be made on the basis of one or more similarities in polarity, charge, solubility, hydrophobicity, hydrophilicity, and/or the amphipathic nature of the residues, as long as the biological or immunological activity of LIPAM is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” can refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid. Amplification may be carried out using polymerase chain reaction (PCR) technologies or other nucleic acid amplification technologies well known in the art.

The term “antagonist” refers to a molecule which inhibits or attenuates the biological activity of LIPAM. Antagonists may include proteins such as antibodies, anticalins, nucleic acids, carbohydrates, small molecules, or any other compound or composition which modulates the activity of LIPAM either by directly interacting with LIPAM or by acting on components of the biological pathway in which LIPAM participates.

The term “antibody” refers to intact immunoglobulin molecules as well as to fragments thereof, such as Fab, F(ab′)₂, and Fv fragments, which are capable of binding an epitopic determinant. Antibodies that bind LIPAM polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment); described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker (Brody, E. N. and L. Gold (2000) J. Biotechnol. 74:5-13).

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic LIPAM, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide” and a “composition comprising a given polypeptide” can refer to any composition containing the given polynucleotide or polypeptide. The composition may comprise a dry formulation or an aqueous solution. Compositions comprising polynucleotides encoding TUPAM or fragments of LIPAM may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (Accelrys, Burlington Mass.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of LIPAM or a polynucleotide encoding LIPAM which can be identical in sequence to, but shorter in length than, the parent sequence. A fragment may comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other pulposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:22-42 can comprise a region of unique polynucleotide sequence that specifically identifies SEQ ID NO:22-42, for example, as distinct from any other sequence in the genome from which the fragment was obtained. A fragment of SEQ ID NO:22-42 can be employed in one or more embodiments of methods of the invention, for example, in hybridization and amplification technologies and in analogous methods that distinguish SEQ ID NO:22-42 from related polynucleotides. The precise length of a fragment of SEQ ID NO:22-42 and the region of SEQ ID NO:22-42 to which the fragment corresponds are routinely determinable by one of ordinary skill in the art based on the intended purpose for the fragment.

A fragment of SEQ ID NO:1-21 is encoded by a fragment of SEQ ID NO:22-42. A fragment of SEQ ID NO:1-21 can comprise a region of unique amino acid sequence that specifically identifies SEQ ID NO:1-21. For example, a fragment of SEQ ID NO:1-21 can be used as an immunogenic peptide for the development of antibodies that specifically recognize SEQ ID NO:1-21. The precise length of a fragment of SEQ ID NO:1-21 and the region of SEQ ID NO:1-21 to which the fragment corresponds can be determined based on the intended purpose for the fragment using one or more analytical methods described herein or otherwise known in the art.

A “full length” polynucleotide is one containing at least a translation initiation codon (e.g., methionine) followed by an open reading frame and a translation termination codon. A “full length” polynucleotide sequence encodes a “full length” polypeptide sequence.

“Homology” refers to sequence similarity or, alternatively, sequence identity, between two or more polynucleotide sequences or two or more polypeptide sequences.

The terms “percent identity” and “% identity,” as applied to polynucleotide sequences, refer to the percentage of identical nucleotide matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.); CLUSTAL V is described in Higgins, D. G. and P. M. Sharp (1989; CABIOS 5:151-153) and in Higgins, D. G. et al. (1992; CABIOS 8:189-191). For pairwise alignments of polynucleotide sequences, the default parameters are set as follows: Ktuple=2, gap penalty=5, window=4, and “diagonals saved”=4. The “weighted” residue weight table is selected as the default.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990)J. Mol. Biol. 215:403-410), which is available from several sources, including the NCBI, Bethesda, Md., and on the Internet at ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at ncbi.nlm.nih.gov/gorf/b12.html. The “BLAST 2 Sequences” tool can be used for both blastn and blastp (discussed below). BLAST programs are commonly used with gap and other parameters set to default settings. For example, to compare two nucleotide sequences, one may use blastm with the “BLAST 2 Sequences” tool Version 2.0.12 (Apr. 21, 2000) set at default parameters. Such default parameters may be, for example:

Matrix: BLOSUM62

Reward for match: 1

Penalty for mismatch: −2

Open Gap: 5 and Extension Gap: 2 penalties

Gap x drop-off. 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of identical residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. The phrases “percent similarity” and “% similarity,” as applied to polypeptide sequences, refer to the percentage of residue matches, including identical residue matches and conservative substitutions, between at least two polypeptide sequences aligned using a standardized algorithm. In contrast, conservative substitutions are not included in the calculation of percent identity between polypeptide sequences.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table.

Alternatively the NCBI BLAST software suite may be used. For example, for a pairwise comparison of two polypeptide sequences, one may use the “BLAST 2 Sequences” tool Version 2.0.12 (Apr. 21, 2000) with blastp set at default parameters. Such default parameters may be, for example:

Matrix: BLOSUM62

Open Gap: 11 and Extension Gap: 1 penalties

Gap x-drop-off 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. and D. W. Russell (2001; Molecular Cloning: A Laboratory Manual, 3rd ed., vol. 1-3, Cold Spring Harbor Press, Cold Spring Harbor N.Y., ch. 9).

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such, blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acids by virtue of the formation of hydrogen bonds between complementary bases. A hybridization complex may be formed in solution (e.g., C₀t or R₀t analysis) or formed between one nucleic acid present in solution and another nucleic acid immobilized on a solid support (e.g., paper, membranes, filters, chips, pins or glass slides, or any other appropriate substrate to which cells or their nucleic acids have been fixed).

The words “insertion” and “addition” refer to changes in an amino acid or polynucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of LIPAM which is capable of eliciting an immune response when introduced into a living organism, for example, a mammal. The term “immunogenic fragment” also includes any polypeptide or oligopeptide fragment of LIPAM which is useful in any of the antibody production methods disclosed herein or known in the art.

The term “microarray” refers to an arrangement of a plurality of polynucleotides, polypeptides, antibodies, or other chemical compounds on a substrate.

The terms “element” and “array element” refer to a polynucleotide, polypeptide, antibody; or other chemical compound having a unique and defined position on a microarray.

The term “modulate” refers to a change in the activity of LIPAM. For example, modulation may cause an increase or a decrease in protein activity, binding characteristics, or any other biological, functional, or immunological properties of LIPAM.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an LIPAM may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of LIPAM.

“Probe” refers to nucleic acids encoding LIPAM, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in, for example, Sambrook, J. and D. W. Russell (2001; Molecular Cloning: A Laboratory Manual, 3rd ed., vol. 1-3, Cold Spring Harbor Press, Cold Spring Harbor N.Y.), Ausubel, F. M. et al. (1999; Short Protocols in Molecular Biology, 4^{th} ed., John Wiley & Sons, New York N.Y.), and Innis, M. et al. (1990; PCR Protocols. A Guide to Methods and Applications, Academic Press, San Diego Calif.). PCR primer pairs can be derived from a known sequence, for example, by using computer programs intended for that purpose such as Primer (Version 0.5, 1991, Whitehead Institute for Biomedical Research, Cambridge Mass.).,

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook and Russell (supra). The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term “sample” is used in its broadest sense. A sample suspected of containing LIPAM, nucleic acids encoding LIPAM, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least about 60% free, preferably at least about 75% free, and most preferably at least about 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a

### THE INVENTION

Various embodiments of the invention include new human lipid-associated molecules (LIPAM), the polynucleotides encoding LIPAM, and the use of these compositions for the diagnosis, treatment, or prevention of cancer, cardiovascular, neurological, autoimmune/inflammatory, and gastrointestinal disorders, and disorders of lipid metabolism.

Table 1 summarizes the nomenclature for the full length polynucleotide and polypeptide embodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown. Column 6 shows the Incyte ID numbers of physical, full length clones corresponding to the polypeptide and polynucleotide sequences of the invention. The full length clones encode polypeptides which have at least 95% sequence identity to the polypeptide sequences shown in column 3.

Table 2 shows sequences with homology to polypeptide embodiments of the invention as identified by BLAST analysis against the GenBank protein (genpept) database and the PROTEONE database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog and the PROTEOME database identification numbers (PROTEOME ID NO:) of the nearest PROTEOME database homologs. Column 4 shows the probability scores for the matches between each polypeptide and its homolog(s). Column 5 shows the annotation of the GenBank and PROTEOME database homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows amino acid residues comprising signature sequences, domains, motifs, potential phosphorylation sites, and potential glycosylation sites. Column 5 shows analytical methods for protein structure/function analysis and in some cases, searchable databases to which the analytical methods were applied.

Together, Tables 2 and 3 summarize the properties of polypeptides of the invention, and these properties establish that the claimed polypeptides are lipid-associated molecules. For example, SEQ recombinant virus. In another embodiment, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook and Russell (supra).

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 7, 1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing during mRNA processing. The corresponding polypeptide may possess additional functional domains orllack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity or sequence similarity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity or sequence similarity over a certain defined length of one of the polypeptides. ID NO:3 is 97% identical, from residue D66 to residue L248 to human surfactant apoprotein 18 precursor (GenBank ID g33828) as determined by the Basic Local Alignment Search Tool (BLAST). (See Table 2.) The BLAST probability score is 4.8e-97, which indicates the probability of obtaining the observed polypeptide sequence alignment by chance. SEQ ID NO:3 also has homology to proteins that are localized to the alveolar region, and are surfactant proteins, as determined by BLAST analysis using the PROTEOME database. SEQ ID NO:3 also contains saposin and surfactant domains as determined by searching for statistically significant matches in the hidden Markov model (HMM)-based PFAM and SMART databases of conserved protein families/domains. (See Table 3.) The foregoing provides evidence that SEQ ID NO:3 is a surfactant type molecule. In an alternative example, SEQ ID NO:6 is 99% identical, from residue M1 to residue K250, and 96% identical, from residue S243 to residue S433, to human cholesteryl ester transfer protein precursor (GenBank ID g180260) as determined by the Basic Local Alignment Search Tool (BLAST). (See Table 2.) The BLAST probability'score is 2.9e-223 for both examples above, which indicates the probability of obtaining the observed polypeptide sequence alignment by chance. SEQ ID NO:6 also has homology to proteins that are cholesteryl ester transfer proteins, as determined by BLAST analysis using the PROTEOME database. SEQ ID NO:6 also contains an LBP/BPI/CETP family, N-terminal domain and a LBP/BPI/CETP family, C-terminal domain as determined by searching for statistically significant matches in the hidden Markov model (HMM)-based PFAM database of conserved protein families/domains. SEQ ID NO:6 also contains a BPI/LBP/CETP, N-terminal domain and a BPI/LBP/CETP family, C-terminal domain as determined by searching for statistically significant matches in the hidden Markov model (FMM)-based SMART database of conserved protein families/domains. (See Table 3.) Data from BLIMPS, MOTIFS, and PROFILESCAN analyses, and BLAST analyses against the PRODOM and DOMO databases, provide further corroborative evidence that SEQ ID NO:6 is a cholesteryl ester transfer protein. In an alternative example, SEQ ID NO:9 is 99% identical, from residue L23 to residue L619 and 100% identical, from residue M1 to residue S26, to human dihydroxyacetone phosphate acyltransferase (DHAPAT), also known as glyceronephosphate O-acyltransferase (GenBank ID g10443718) as determined by the Basic Local Alignment Search Tool (BLAST). (See Table 2.) The BLAST probability score is 0.0, which indicates the probability of obtaining the observed polypeptide sequence alignment by chance. SEQ ID NO:9 also has homology to proteins that are localized to the peroxisome, function as transferases, and are glyceronephosphate O-acyltransferases, as determined by BLAST analysis using the PROTEOME database. SEQ ID NO:9 also contains an acyltransferase domain as determined by searching for statistically significant matches in the hidden Markov model (HMM)-based PFAM database of conserved protein families/domains. Further, SEQ ID NO:9 contains a 1-acyl-sn-glycerol-3-phosphate acyltransferases domain as determined by searching for statistically significant matches in the hidden Markov model (HMM)-based TIGRFAM database of conserved protein families/domains. (See Table 3.) Data from BLAST analyses against the PRODOM and DOMO databases provide further corroborative evidence that SEQ ID NO:9 is a splice variant of glyceronephosphate O-acyltransferase. In an alternative example, SEQ ID NO:14 is 94% identical, from residue M1 to residue L234, and 90% identical, from residue G217 to residue L304, to human peroxisomal enoyl-CoA hydratase-like protein (GenBank ID g564065) as determined by the Basic Local Alignment Search Tool (BLAST). (See Table 2.) The BLAST probability score is 4.8e-152, which indicates the probability of obtaining the observed polypeptide sequence alignment by chance. SEQ ID NO:14 also has homology to proteins that are localized to the peroxisome, have peroxisomal beta-oxidation function, and are peroxisomal enoyl-CoA hydratase-like proteins, as determined by BLAST analysis using the PROTEOME database. SEQ ID NO:14 also contains an enoyl-CoA hydratase/isomerase family domain as determined by searching for statistically significant matches in the hidden Markov model (HMM)-based PFAM database of conserved protein families/domains. (See Table 3.) Data from BLIMPS, MOTIFS, and PROFILESCAN analyses, and BLAST analyses against the PRODOM and DOMO databases, provide further corroborative evidence that SEQ ID NO:14 is an enoyl-CoA hydratase/isomerase. In an alternative example, SEQ ID NO:17 is 97% identical, from residue M1 to residue G77, and 100% identical, from residue G97 to residue Q419 to human lysosomal acid lipase/cholesteryl esterase (GenBank ID g187152) as determined by the Basic Local Alignment Search Tool (BLAST). (See Table 2.) The BLAST probability score is 4.5e-221, which indicates the probability of obtaining the observed polypeptide sequence alignment by chance. SEQ ID NO:17 also has homology to proteins that are localized to lysosomes or vacuoles, deacylate cholesteryl and triacylglyceryl ester core lipids from low density lipoproteins, are hydrolases, and mutations in are associated with Wolman disease and cholesteryl ester storage diseases, as determined by BLAST analysis using the PROTEOME database. SEQ ID NO:17 also contains an alpha/beta hydrolase fold as determined by searching for statistically significant matches in the hidden Markov model (HMM)-based PFAM database of conserved protein families/domains. (See Table 3.) Data from BLIMPS and MOTIFS analyses, and BLAST analyses against the PRODOM and DOMO databases, provide further corroborative evidence that SEQ ID NO:17 is a lysosomal lipase/cholesteryl esterase. In an alternative example, SEQ ID NO:20 is 91% identical, from residue H164 to residue P426, to human endothelial lipase (GenBank ID g4836419) as determined by the Basic Local Alignment Search Tool (BLAST). (See Table 2.) The BLAST probability score is 9.6e-236, which indicates the probability of obtaining the observed polypeptide sequence alignment by chance. SEQ ID NO:20 also has homology to endothelial-derived lipase (lipase G), a member of the triacylglycerol lipase family which catalyzes the hydrolysis of phosphatidylcholine, and may play a role in lipoprotein metabolism, inflammation, and development of vascular diseases like atherosclerosis, as determined by BLAST analysis using the PROTEOME database. SEQ ID NO:20 also contains PLAT/LM2, lipase, and lipoxygenase homology 2 (beta barrel) domains as determined by searching for statistically significant matches in the hidden Markov model (HMM)-based PFAMISMART databases of conserved protein families/domains. (See Table 3.) Data from BLVIPS, MOTIFS, and PROFILESCAN analyses, and BLAST analyses against the PRODOM and DOMO databases, provide further corroborative evidence that SEQ ID NO:20 is a lipase. In an alternative example, SEQ ID NO:21 is 99% identical, from residue S55 to residue Q902, to human phospholipase C beta 4 (GenBank ID g762826) as determined by the Basic Local Alignment Search Tool (BLAST). (See Table 2.) The BLAST probability score is 0.0, which indicates the probability of obtaining the observed polypeptide sequence alignment by chance. SEQ ID NO:21 also has homology to proteins that hydrolyze phosphatidylinositol 4,5-bisphosphate to the second messengers 1,4,5-trisphosphate and diacylglycerol, and are phospholipases, as determined by BLAST analysis using the PROTEOME database. SEQ ID NO:21 also contains a C2 domain, phosphatidylinositol-specific phospholipase C (X and Y domains), a protein kinase C conserved region 2 (CalB) domain, and phospholipase C catalytic domain (part) domains X and Y as determined by searching for statistically significant matches in the hidden Markov model (HMM)-based PFAM and SMART databases of conserved protein families/domains. (See Table 3.) Data from BLUMPS analyses, and BLAST analyses against the PRODOM and DOMO databases, provide further corroborative evidence that SEQ ID NO:21 is a phospholipase. SEQ ID NO:1-2, SEQ ID NO:4-5, SEQ ID NO:7-8, SEQ ID NO:10-13, SEQ ID NO:15-16, and SEQ ID NO:18-19 were analyzed and annotated in a similar manner. The algorithms and parameters for the analysis of SEQ ID NO:1-21 are described in Table 7.

As shown in Table 4, the full length polynucleotide embodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide embodiments, and of fragments of the polynucleotides which are useful, for example, in hybridization or amplification technologies that identify SEQ ID NO:22-42 or that distinguish between SEQ ID NO:22-42 and related polynucleotides.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or “NT”) or the NCBI RefSeq Protein Sequence Records (ie., those sequences including the designation “NP”). Alternatively, the polynucleotide fragments described in column 2 may refer to assemblages of both cDNA and Genscan-predicted exons brought together by an “exon stitching” algorithm. For example, a polynucleotide sequence identified as FL_XXXXXX_N_{1—}N_{2—}YYYYY_N_{3—}N₄ represents a “stitched” sequence in which XXXXXX is the identification number of the cluster of sequences to which the algorithm was applied, and YYYYY is the number of the prediction generated by the algorithm, and N_{1,2,3 . . .} , if present, represent specific exons that may have been manually edited during analysis (See Example V). Alternatively, the polynucleotide fragments in column 2 may refer to assemblages of exons brought together by an “exon-stretching” algorithm. For example, a polynucleotide sequence identified as FLXXXXXX_gAAAAA_gBBBBB_{—}1_N is a “stretched” sequence, with XXXXX being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (ie., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, GFG, | Exon prediction from genomic sequences using, |
| ENST | for example, GENSCAN (Stanford University, |
| | CA, USA) or FGENES (Computer Genomics |
| | Group, The Sanger Centre, Cambridge, UK). |
| GBI | Hand-edited analysis of genomic sequences. |
| FL | Stitched or stretched genomic sequences (see |
| | Example V). |
| INCY | Full length transcript and exon prediction from |
| | mapping of EST sequences to the genome. |
| | Genomic location and EST composition |
| | data are combined to predict the exons and |
| | resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were used to construct the cDNA libraries shown in Table 5 are described in Table 6.

Table 8 shows single nucleotide polymorphisms (SNPs) found in polynucleotide sequences of the invention, along with allele frequencies in different human populations. Columns 1 and 2 show the polynucleotide sequence identification number (SEQ ID NO:) and the corresponding Incyte project identification number (PID) for polynucleotides of the invention. Column 3 shows the Incyte identification number for the EST in which the SNP was detected (EST ID), and column 4 shows the identification number for the SNP (SNP ID). Column 5 shows the position within the EST sequence at which the SNP is located (EST SNP), and column 6 shows the position of the SNP within the full-length polynucleotide sequence (CB1 SNP). Column 7 shows the allele found in the EST sequence. Columns 8 and 9 show the two alleles found at the SNP site. Column 10 shows the amino acid encoded by the codon including the SNP site, based upon the allele found in the EST. Columns 11-14 show the frequency of allele 1 in four different human populations. An entry of n/d (not detected) indicates that the frequency of allele 1 in the population was too low to be detected, while n/a (not available) indicates that the allele frequency was not determined for the population.

The invention also encompasses LIPAM variants. Various embodiments of LIPAM variants can have at least about 80%, at least about 90%, or at least about 95% amino acid sequence identity to the LIPAM amino acid sequence, and can contain at least one functional or structural characteristic of LIPAM.

Various embodiments also encompass polynucleotides which encode LIPAM. In a particular embodiment, the invention encompasses a polynucleotide sequence comprising a sequence selected from the group consisting of SEQ ID NO:22-42, which encodes LIPAM. The polynucleotide sequences of SEQ ID NO:22-42, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding LIPAM. In particular, such a variant polynucleotide will have at least about 70%, or alternatively at least about 85%, or even at least about 95% polynucleotide sequence identity to a polynucleotide encoding LIPAM. A particular aspect of the invention encompasses a variant of a polynucleotide comprising a sequence selected from the group consisting of SEQ ID NO:22-42 which has at least about 70%, or alternatively at least about 85%, or even at least about 95% polynucleotide sequence identity to a nucleic acid sequence selected from the group consisting of SEQ ID NO:22-42. Any one of the polynucleotide variants described above can encode a polypeptide which contains at least one functional or structural characteristic of LIPAM.

In addition, or in the alternative, a polynucleotide variant of the invention is a splice variant of a polynucleotide encoding LIPAM. A splice variant may have portions which have significant sequence identity to a polynucleotide encoding LIPAM, but will generally have a greater or lesser number of nucleotides due to additions or deletions of blocks of sequence arising from alternate splicing during mRNA processing. A splice variant may have less than about 70%, or alternatively less than about 60%, or alternatively less than about 50% polynucleotide sequence identity to a polynucleotide encoding LIPAM over its entire length; however, portions of the splice variant will have at least about 70%, or alternatively at least about 85%, or alternatively at least about 95%, or alternatively 100% polynucleotide sequence identity to portions of the polynucleotide encoding LIPAM. For example, a polynucleotide comprising a sequence of SEQ ID NO:25 and a polynucleotide comprising a sequence of SEQ ID NO:26 are splice variants of each other; a polynucleotide comprising a sequence of SEQ ID NO:31 and a polynucleotide comprising a sequence of SEQ ID NO:32 are splice variants of each other; and a polynucleotide comprising a sequence of SEQ ID NO:36 and a polynucleotide comprising a sequence of SEQ ID NO:37 are splice variants of each other. Any one of the splice variants described above can encode a polypeptide which contains at least one functional or structural characteristic of LIPAM.

It will be appreciated by those skilled in the art that as a result of the degeneracy of the genetic code, a multitude of polynucleotide sequences encoding LIPAM, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring LIPAM, and all such variations are to be considered as being specifically disclosed.

Although polynucleotides which encode LIPAM and its variants are generally capable of hybridizing to polynucleotides encoding naturally occurring LIPAM under appropriately selected conditions of stringency, it may be advantageous to produce polynucleotides encoding LIPAM or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding LIPAM and its derivatives without altering the encoded amino acid sequences include the production of RNA transcripts having more desirable properties, such as a greater half-life, than transcripts produced from the naturally occurring sequence.

The invention also encompasses production of polynucleotides which encode LIPAM and LIPAM derivatives, or fragments thereof, entirely by synthetic chemistry. After production, the synthetic polynucleotide may be inserted into any of the many available expression vectors and cell systems using reagents well known in the art. Moreover, synthetic chemistry may be used to introduce mutations into a polynucleotide encoding LIPAM or any fragment thereof.

Embodiments of the invention can also include polynucleotides that are capable of hybridizing to the claimed polynucleotides, and, in particular, to those having the sequences shown in SEQ ID NO:22-42 and fragments thereof, under various conditions of stringency (Wahl, G. M. and S. L. Berger (1987) Methods Enzymol. 152:399-407; Kimmel, A. R. (1987) Methods Enzymol. 152:507-511). Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art (Ausubel et al., supra, ch. 7; Meyers, R. A. (1995) Molecular Biolog and Biotechnology, Wiley VCH, New York N.Y., pp. 856-853).

The nucleic acids encoding LIPAM may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector (Sarkar, G. (1993) PCR Methods Applic. 2:318-322). Another method, inverse PCR, uses primers that extend in divergent directions to amplify unknown sequence from a circularized template. The template is derived from restriction fragments comprising a known genomic locus and surrounding sequences (Triglia, T. et al. (1988) Nucleic Acids Res. 16:8186). A third method, capture PCR, involves PCR amplification of DNA fragments adjacent to known sequences in human and yeast artificial chromosome DNA (Lagerstrom, M. et al. (1991) PCR Methods Applic. 1:111-119). In this method, multiple restriction enzyme digestions and ligations may be used to insert an engineered double-stranded sequence into a region of unknown sequence before performing PCR. Other methods which may be used to retrieve unknown sequences are known in the art (Parker, J. D. et al. (1991) Nucleic Acids Res. 19:3055-3060). Additionally, one may use PCR, nested-primers, and PROMOTERFINDER libraries (BD Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotides or fragments thereof which encode LIPAM may be cloned in recombinant DNA molecules that direct expression of LIPAM, or fragments or functional equivalents thereof, in appropriate host cells. Due to the inherent degeneracy of the genetic code, other polynucleotides which encode substantially the same or a functionally equivalent polypeptides may be produced and used to express LIPAM.

The polynucleotides of the invention can be engineered using methods generally known in the art in order to alter LIPAM-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of LIPAM, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding LIPAM may be synthesized, in whole or in part, using one or more chemical methods well known in the art (Caruthers, M. H. et al. (1980) Nucleic Acids Symp. Ser. 7:215-223; Horn, T. et al. (1980) Nucleic Acids Symp. Ser. 7:225-232). Alternatively, LIPAM itself or a fragment thereof may be synthesized using chemical methods known in the art. For example, peptide synthesis can be performed using various solution-phase or solid-phase techniques (Creighton, T. (1984) Proteins, Structures and Molecular Properties, W H Freeman, New York N.Y., pp. 55-60; Roberge, J. Y. et al. (1995) Science 269:202-204). Automated synthesis may be achieved using the ABI 431A peptide synthesizer (Applied Biosystems). Additionally, the amino acid sequence of LIPAM, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography (Chiez, R. M. and F. Z. Regnier (1990) Methods Enzymol. 182:392-421). The composition of the synthetic peptides may be confirmed by amino acid analysis or by sequencing (Creighton, supra, pp. 28-53).

In order to express a biologically active LIPAM, the polynucleotides encoding LIPAM or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotides encoding LIPAM. Such elements may vary in their strength and specificity. Specific initiation signals-may also be used to achieve more efficient translation of polynucleotides encoding LIPAM. Such signals include the ATG initiation codon and adjacent sequences, e.g. the Kozak sequence. In cases where a polynucleotide sequence encoding LIPAM and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (Scharf, D. et al. (1994) Results Probl. Cell Differ. 20:125-162).

Methods which are well known to those skilled in the art may be used to construct expression vectors containing polynucleotides encoding LIPAM and appropriate transcriptional and translational control elements. These methods include in vitro recombinant DNA techniques, synthetic techniques, and in vivo genetic recombination (Sambrook and Russell, supra, ch. 1-4, and 8; Ausubel et al., supra, ch. 1, 3, and 15).

A variety of expression vector/host systems may be utilized to contain and express polynucleotides encoding LIPAM. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TV) or with bacterial expression vectors (e.g., Ti or pBR322 plasmids); or animal cell systems (Sambrook and Russell, supra; Ausubel et al., supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; Harrington, J. J. et al. (1997) Nat. Genet. 15:345-355). Expression vectors derived from retroviruses, adenoviruses, or herpes or vaccinia viruses, or from various bacterial plasmids, may be used for delivery of polynucleotides to the targeted organ, tissue, or cell population (Di Nicola, M. et al. (1998) Cancer Gen. Ther. 5:350-356; Yu, M. et al. (1993) Proc. Natl. Acad. Sci. USA 90:6340-6344; Buller, R. M. et al. (1985) Nature 317:813-815; McGregor,. D. P. et al. (1994) Mol. Immunol. 31:219-226; Verma, I. M. and N. Somia (1997) Nature 389:239-242). The invention is not limited by the host cell employed.

In bacterial systems, a number of cloning and expression vectors may be selected depending upon the use intended for polynucleotides encoding LIPAM. For example, routine cloning, subcloning, and propagation of polynucleotides encoding LIPAM can be achieved using a multifunctional E. coli vector such as PBLUESCRIPT (Stratagene, La Jolla Calif.) or PSPORT1 plasmid (Invitrogen). Ligation of polynucleotides encoding LIPAM into the vector's multiple cloning site disrupts the lacZ. gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence (Van Heeke, G. and S. M. Schuster (1989) J. Biol. Chem. 264:5503-5509). When large quantities of LIPAM are needed, e.g. for the production of antibodies, vectors which direct high level expression of LIPAM may be used. For example, vectors containing the strong, inducible SP6 or T7 bacteriophage promoter may be used.

Yeast expression systems may be used for production of LIPAM. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign polynucleotide sequences into the host genome for stable propagation (Ausubel et al., supra; Bitter, G. A. et al. (1987) Methods Enzymol. 153:516-544; Scorer, C. A. et al. (1994) Bio/Technology 12:181-184).

Plant systems may also be used for expression of LIPAM. Transcription of polynucleotides encoding LIPAM may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; Winter, J. et al. (1991) Results Probl. Cell Differ. 17:85-105). These constructs can be introduced into plant cells by direct DNA transformation or pathogen-mediated transfection (The McGraw Hill Yearbook of Science and Technology (1992) McGraw Hill, New York N.Y., pp. 191-196).

In mammalian cells, a number of viral-based expression systems may be utilized. In cases where an adenovirus is used as an expression vector, polynucleotides encoding LIPAM may be ligated into an adenovirus transcription/translation complex consisting of the late promoter and tripartite leader sequence. Insertion in a non-essential E1 or E3 region of the viral genome may be used to obtain infective virus which expresses LIPAM in host cells (Logan, J. and T. Shenk (1984) Proc. Natl. Acad. Sci. USA 81:3655-3659). In addition, transcription enhancers, such as the Rous sarcoma virus (RSV) enhancer, may be-used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes (Hairington, J. J. et al. (1997) Nat. Genet. 15:345-355).

For long term production of recombinant proteins in mammalian systems, stable expression of LIPAM in cell lines is preferred. For example, polynucleotides encoding LIPAM can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively (Wigler, M. et al. (1977) Cell 11:223-232; Lowy, I. et al. (1980) Cell 22:817-823). Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively (Wigler, M. et al. (1980) Proc. Natl. Acad. Sci. USA 77:3567-3570; Colbere-Garapin, F. et al. (1981) J. Mol. Biol. 150:1-14). Additional selectable genes have been described, e.g., trpB and hisD, which alter cellular requirements for metabolites (Hartman, S. C. and R. C. Mulligan (1988) Proc. Natl. Acad. Sci. USA 85:8047-8051). Visible markers, e.g., anthocyanins, green fluorescent proteins (GFP; BD Clontech), β-glucuronidase and its substrate β-glucuronide, or luciferase and its substrate luciferin may be used. These markers can be used not only to identify transformants, but also to quantify the amount of transient or stable protein expression attributable to a specific vector system (Rhodes, C. A. (1995) Methods Mol. Biol. 55:121-131).

Although the presence/absence of marker gene expression suggests that the gene of interest is also present, the presence and expression of the gene may need to be confirmed. For example, if the sequence encoding LIPAM is inserted within a marker gene sequence, transformed cells containing polynucleotides encoding LIPAM can be identified by the absence of marker gene function. Alternatively, a marker gene can be placed in tandem with a sequence encoding LIPAM under the control of a single promoter. Expression of the marker gene in response to induction or selection usually indicates expression of the tandem gene as well.

In general, host cells that contain the polynucleotide encoding LIPAM and that express LIPAM may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of LIPAM using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on LIPAM is preferred, but a competitive binding assay may be employed. These and other assays are well known in the art (Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; Pound, J. D. (1998) Immunochemical Protocols, Humana Press, Totowa N.J.).

A wide variety of labels and conjugation techniques are known by those skilled in the art and may be used in various nucleic acid and amino acid assays. Means for producing labeled hybridization or PCR probes for detecting sequences related to polynucleotides encoding LIPAM include oligolabeling, nick translation, end-labeling, or PCR amplification using a labeled nucleotide.

Alternatively, polynucleotides encoding LIPAM, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Biosciences, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with polynucleotides encoding LIPAM may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode LIPAM may be designed to contain signal sequences which direct secretion of LIPAM through a prokaryotic or eukaryotic cell membrane.

In addition, a host cell strain may be chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant polynucleotides encoding LIPAM may be ligated to a heterologous sequence resulting in translation of a fusion protein in any of the aforementioned host systems. For example, a chimeric LIPAM protein containing a heterologous moiety that can be recognized by a commercially available antibody may facilitate the screening of peptide libraries for inhibitors of LIPAM activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the LIPAM encoding sequence and the heterologous protein sequence, so that LIPAM may be cleaved away from the heterologous moiety following purification. Methods for fusion protein expression and purification are discussed in Ausubel et al. (supra, ch. 10 and 16). A variety of commercially available kits may also be used to facilitate expression and purification of fusion proteins.

In another embodiment, synthesis of radiolabeled LIPAM may be achieved in vitro using the TNT rabbit reticulocyte lysate or wheat germ extract system (Promega). These systems couple transcription and translation of protein-coding sequences operably associated with the T7, T3, or SP6 promoters. Translation-takes place in the presence of a radiolabeled amino acid precursor, for example, ³⁵S-methionine.

LIPAM, fragments of LIPAM, or variants of LIPAM may be used to screen for compounds that specifically bind to LIPAM. One or more test compounds may be screened for specific binding to LIPAM. In various embodiments, 1, 2, 3, 4, 5, 10, 20, 50, 100, or 200 test compounds can be screened for specific binding to LIPAM. Examples of test compounds can include antibodies, anticalins, oligonucleotides, proteins (e.g., ligands or receptors), or small molecules.

In related embodiments, variants of LIPAM can be used to screen for binding of test compounds, such as antibodies, to LIPAM, a variant of LIPAM, or a combination of LIPAM and/or one or more variants LIPAM. In an embodiment, a variant of LIPAM can be used to screen for compounds that bind to a variant of LIPAM, but not to LIPAM having the exact sequence of a sequence of SEQ ID NO:1-21. LIPAM variants used to perform such screening can have a range of about 50% to about 99% sequence identity to LIPAM, with various embodiments having 60%, 70%, 75%, 80%, 85%, 90%, and 95% sequence identity.

In an embodiment, a compound identified in a screen for specific binding to LIPAM can be closely related to the natural ligand of LIPAM, e.g., a ligand or fragment thereof, a natural substrate, a structural or functional mimetic, or a natural binding partner (Coligan, J. E. et al. (1991) Current Protocols in Immunology 1(2):Chapter 5). In another embodiment, the compound thus identified can be a natural ligand of a receptor LIPAM (Howard, A. D. et al. (2001) Trends Pharmacol. Sci.22:132-140; Wise, A. et al. (2002) Drug Discovery Today 7:235-246).

In other embodiments, a compound identified in a screen for specific binding to LIPAM can be closely related to the natural receptor to which LIPAM binds, at least a fragment of the receptor, or a fragment of the receptor including all or a portion of the ligand binding site or binding pocket. For example, the compound may be a receptor for LIPAM which is capable of propagating a signal, or a decoy receptor for LIPAM which is not capable of propagating a signal (Ashkenazi, A. and V. M. Divit (1999) Curr. Opin. Cell Biol. 11:255-260; Mantovani, A. et al. (2001) Trends Immunol. 22:328-336). The compound can be rationally designed using known techniques. Examples of such techniques include those used to construct the compound etanercept (ENBREL; Amgen Inc., Thousand Oaks Calif.), which is efficacious for treating rheumatoid arthritis in humans. Etanercept is an engineered p75 tumor necrosis factor (TNF) receptor dimer linked to the Fc portion of human IgG₁ (Taylor, P. C. et al. (2001) Curr. Opin. Immunol. 13:611-616).

In one embodiment, two or more antibodies having similar or, alternatively, different specificities can be screened for specific binding to LIPAM, fragments of LIPAM, or variants of LIPAM. The binding specificity of the antibodies thus screened can thereby be selected to identify particular fragments or variants of LIPAM. In one embodiment, an antibody can be selected such that its binding specificity allows for preferential identification of specific fragments or variants of LIPAM. In another embodiment, an antibody can be selected such that its binding specificity allows for preferential diagnosis of a specific disease or condition having increased, decreased, or otherwise abnormal production of LIPAM.

In an embodiment, anticalins can be screened for specific binding to LIPAM, fragments of LIPAM, or variants of LIPAM. Anticalins are ligand-binding proteins that have been constructed based on a lipocalin scaffold (Weiss, G. A. and H. B. Lowman (2000) Chem. Biol. 7:R177-R184; Skerra, A. (2001) J. Biotechnol. 74:257-275Y. The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit LIPAM involves producing appropriate cells which express LIPAM, either as a secreted protein or on the cell membrane. Preferred cells can include cells from mammals, yeast, Drosophila, or E. coli. Cells expressing LIPAM or cell membrane fractions which contain LIPAM are then contacted with a test compound and binding, stimulation, or inhibition of activity of either LIPAM or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with LIPAM, either in solution or affixed to a solid support, and detecting the binding of LIPAM to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands (Matthews, D. J. and J. A. Wells. (1994) Chem. Biol. 1:25-30). In another related embodiment, one or more amino acid substitutions can be introduced into a polypeptide compound (such as a ligand) to improve or alter its ability to bind to its natural receptors (Cunningham, B. C. and J. A. Wells (1991) Proc. Natl. Acad. Sci. USA 88:3407-3411; Lowman, H. B. et al. (1991)J. Biol. Chem. 266:10982-10988).

LIPAM, fragments of LIPAM, or variants of LIPAM may be used to screen for compounds that modulate the activity of LIPAM. Such compounds may include agonists, antagonists, or partial or inverse agonists. In one embodiment, an assay is performed under conditions permissive for LIPAM activity, wherein LIPAM is combined with at least one test compound, and the activity of LIPAM in the presence of a test compound is compared with the activity of LIPAM in the absence of the test compound. A change in the activity of LIPAM in the presence of the test compound is indicative of a compound that modulates the activity of LIPAM. Alternatively, a test compound is combined with an, in vitro or cell-free system comprising LIPAM under conditions suitable for LIPAM activity, and the assay is performed. In either of these assays, a test compound which modulates the activity of LIPAM may do so indirectly and need not come in direct contact with the test compound. At least one and up to a plurality of test compounds may be screened.

In another embodiment, polynucleotides encoding LIPAM or their mammalian homologs may be “knocked out” in an animal model system using homologous recombination in embryonic stem (ES) cells. Such techniques are well known in the art and are useful for the generation of animal models of human disease (see, e.g., U.S. Pat. No. 5,175,383 and U.S. Pat. No. 5,767,337). For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding LIPAM may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding LIPAM can also be used to create “knockin” humanized animals (pigs) or transgenic animals (mice or rats) to model human disease. With knockin technology, a region of a polynucleotide encoding LIPAM is injected into animal ES cells, and the injected sequence integrates into the animal cell genome. Transformed cells are injected into. blastulae, and the blastulae are implanted as described above. Transgenic progeny or inbred lines are studied and treated with potential pharmaceutical agents to obtain information on treatment of a human disease. Alternatively, a mammal inbred to overexpress LIPAM, e.g., by secreting LIPAM in its milk, may also serve as a convenient source of that protein (Janne, J. et al. (1998) Biotechnol. Annu. Rev. 4:55-74).

THERAPEUTICS

Chemical and structural similarity, e.g., in the context of sequences and motifs, exists between regions of LIPAM and lipid-associated molecules. In addition, examples of tissues expressing LIPAM can be found in Table 6 and can also be found in Example XI. Therefore, LIPAM appears to play a role in cancer, cardiovascular, neurological, autoimmune/inflammatory, and gastrointestinal disorders, and disorders of lipid metabolism. In the treatment of disorders associated with increased LIPAM expression or activity, it is desirable to decrease the expression or activity of LIPAM. In the treatment of disorders associated with decreased LIPAM expression or activity, it is desirable to increase the expression or activity of LIPAM.

Therefore, in one embodiment, LIPAM or a fragment or derivative thereof may be administered to a subject to treat or prevent a disorder associated with decreased expression or activity of LIPAM. Examples of such disorders include, but are not limited to, a cancer, such as adenocarcinoma, leukemia, lymphoma, melanoma, myeloma, sarcoma, teratocarcinoma, and, in particular, cancers of the adrenal gland, bladder, bone, bone marrow, brain, breast, cervix, colon, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a cardiovascular disorder such as arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation, congenital lung anomalies, atelectasis, pulmonary congestion and edema, pulmonary embolism, pulmonary hemorrhage, pulmonary infarction, pulmonary hypertension, vascular sclerosis, obstructive pulmonary disease, restrictive pulmonary disease, chronic obstructive pulmonary disease, emphysema, chronic bronchitis, bronchial asthma, bronchiectasis, bacterial pneumonia, viral and mycoplasmal pneumonia, lung abscess, pulmonary tuberculosis, diffuse interstitial diseases, pneumoconioses, sarcoidosis, idiopathic pulmonary fibrosis, desquamative interstitial pneumonitis, hypersensitivity pneumonitis, pulmonary eosinophilia bronchiolitis obliterans-organizing pneumonia, diffuse pulmonary hemorrhage syndromes, Goodpasture's syndromes, idiopathic pulmonary. hemosiderosis, pulmonary involvement in collagen-vascular disorders, pulmonary alveolar proteinosis, lung tumors, inflammatory and noninflammatory pleural effusions, pneumothorax, pleural tumors, drug-induced lung disease, radiation-induced lung disease, and complications of lung transplantation; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-lakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a gastrointestinal disorder such as dysphagia, peptic esophagitis, esophageal spasm, esophageal stricture, esophageal carcinoma, dyspepsia, indigestion, gastritis, gastric carcinoma, anorexia, nausea, emesis, gastroparesis, antral or, pyloric edema, abdominal angina, pyrosis, gastroenteritis, intestinal obstruction, infections of the intestinal tract, peptic ulcer, cholelithiasis, cholecystitis, cholestasis, pancreatitis, pancreatic carcinoma, biliary tract disease, hepatitis, hyperbilirubinemia, cirrhosis, passive congestion of the liver, hepatoma, infectious colitis, ulcerative colitis, ulcerative proctitis, Crohn's disease, Whipple's disease, Mallory-Weiss syndrome, colonic carcinoma, colonic obstruction, irritable bowel syndrome, short bowel syndrome, diarrhea, constipation, gastrointestinal hemorrhage, acquired immunodeficiency syndrome (AIDS) enteropathy, jaundice, hepatic encephalopathy, hepatorenal syndrome, hepatic steatosis, hemochromatosis, Wilson's disease, alpha₁-antitrypsin deficiency, Reye's syndrome, primary sclerosing cholangitis, liver infarction, portal vein obstruction and thrombosis, centrilobular necrosis, peliosis hepatis, hepatic vein thrombosis, veno-occlusive disease, preeclampsia, eclampsia, acute fatty liver of pregnancy, intrahepatic cholestasis of pregnancy, and hepatic tumors including nodular hyperplasias, adenomas, and carcinomas; and a disorder of lipid metabolism such as fatty liver, cholestasis, primary biliary cirrhosis, carnitine deficiency, carnitine palmitoyltransferase deficiency, myoadenylate deaminase deficiency, hypertriglyceridemia, lipid storage disorders such Fabry's disease, Gaucher's disease, Niemann-Pick's disease, metachromatic leukodystrophy, adrenoleukodystrophy, GM₂ gangliosidosis, and ceroid lipofuscinosis, abetalipoproteinemia, Tangier disease, hyperlipoproteinemia, diabetes mellitus, lipodystrophy, lipomatoses, acute panniculitis, disseminated fat necrosis, adiposis dolorosa, lipoid adrenal hyperplasia, minimal change disease, lipomas, atherosclerosis, hypercholesterolemia, hypercholesterolemia with hypertriglyceridemia, primary hypoalphalipoproteinemia, hypothyroidism, renal disease, liver disease, lecithin:cholesterol acyltransferase deficiency, cerebrotendinous xanthomatosis, sitosterolemia, hypocholesterolemia, Tay-Sachs disease, Sandhoff's disease, hyperlipidemia, hyperlipemia, lipid myopathies, and obesity.

In another embodiment, a vector capable of expressing LIPAM or a fragment or derivative thereof may be administered to a subject to treat or prevent a disorder associated with decreased expression or activity of LIPAM including, but not limited to, those described above.

In a further embodiment, a composition comprising a substantially purified LIPAM in conjunction with a suitable pharmaceutical carrier may be administered to a subject to treat or prevent a disorder associated with decreased expression or activity of LIPAM including, but not limited to, those provided above.

In still another embodiment, an agonist which modulates the activity of LIPAM may be administered to a subject to treat or prevent a disorder associated with decreased expression or activity of LIPAM including, but not limited to, those listed above.

In a further embodiment, an antagonist of LIPAM may be administered to a subject to treat or prevent a disorder associated with increased expression or activity of LIPAM. Examples of such disorders include, but are not limited to, those cancer, cardiovascular, neurological, autoimmune/inflammatory, and gastrointestinal disorders, and disorders of lipid metabolism described above. In one aspect, an antibody which specifically binds LIPAM may be used directly as an antagonist or indirectly as a targeting or delivery mechanism for bringing a pharmaceutical agent to cells or tissues which express LIPAM.

In an additional embodiment, a vector expressing the complement of the polynucleotide encoding LIPAM may be administered to a subject to treat or prevent a disorder associated with increased expression or activity of LIPAM including, but not limited to, those described above.

In other embodiments, any protein, agonist, antagonist, antibody, complementary sequence, or vector embodiments may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of LIPAM may be produced using methods which are generally known in the art. In particular, purified LIPAM may be used to produce antibodies or to screen libraries of pharmaceutical agents to identify those which specifically bind LIPAM. Antibodies to LIPAM may also be generated using methods that are well known in the art. Such antibodies may include, but are not limited to, polyclonal, monoclonal, chimeric, and single chain antibodies, Fab fragments, and fragments produced by a Fab expression library. In an embodiment, neutralizing antibodies (i.e., those which inhibit dimer formation) can be used therapeutically. Single chain antibodies (e.g., from camels or llamas) may be potent enzyme inhibitors and may have application in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with LIPAM or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to LIPAM have an amino acid sequence consisting of at least about 5 amino acids, and generally will consist of at least about 10 amino acids. It is also preferable that these oligopeptides, peptides, or fragments are substantially identical to a portion of the amino acid sequence of the natural protein. Short stretches of LIPAM amino acids may be fused with those of another protein, such as KLH, and antibodies to the chimeric molecule may be produced.

Monoclonal antibodies to LIPAM may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; Cole, S. P. et al. (1984) Mol. Cell Biol. 62:109-120).

In addition, techniques developed for the production of “chimeric antibodies,” such as the splicing of mouse antibody genes to human antibody genes to obtain a molecule with appropriate antigen specificity and biological activity, can be used (Morrison, S. L. et al. (1984) Proc. Natl. Acad. Sci. USA 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce LIPAM-specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. USA 88:10134-10137).

Antibodies may also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. USA 86:3833-3837; Winter, G. et al. (1991) Nature 349:293-299).

Antibody fragments which contain specific binding sites for LIPAM may also be generated. For example, such fragments include, but are not limited to, F(ab′)₂ fragments produced by pepsin digestion of the antibody molecule and Fab fragments generated by reducing the disulfide bridges of the F(ab′)2 fragments. Alternatively, Fab expression libraries may be constructed to allow rapid and, easy identification of monoclonal Fab fragments with the desired specificity (Huse, W. D. et al. (1989) Science 246:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between LIPAM and its specific antibody. A two-site, monoclonal-based immunoassay utilizing monoclonal antibodies reactive to two non-interfering LIPAM epitopes is generally used, but a competitive binding assay may also be: employed (Pound, supra).

Various methods such as Scatchard analysis in conjunction with radioimmunoassay techniques may be used to assess the affinity of antibodies for LIPAM. Affinity is expressed as an association constant, Kₐ, which is defined as the molar concentration of LIPAM-antibody complex divided by the molar concentrations of free antigen and free antibody under equilibrium conditions. The Kₐ determined for a preparation of polyclonal antibodies, which are heterogeneous in their affinities for multiple LIPAM epitopes, represents the average affinity, or avidity, of the antibodies for LIPAM. The Kₐ determined for a preparation of monoclonal antibodies, which are monospecific for a particular LIPAM epitope, represents a true measure of affinity. High-affinity antibody preparations with Kₐ ranging from about 10⁹ to 10¹² L/mole are preferred for use in immunoassays in which the LIPAM-antibody complex must withstand rigorous manipulations. Low-affinity antibody preparations with Kₐ ranging from about 10⁶ to 10⁷ L/mole are preferred for use in immunopurification and similar procedures which ultimately require dissociation of LIPAM, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of LIPAM-antibody complexes. Procedures for evaluating antibody specificity, titer, and avidity, and guidelines for antibody quality and usage in various applications, are generally available (Catty, supra; Coligan et al., supra).

In another embodiment of the invention, polynucleotides encoding LIPAM, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding LIPAM. Such technology is well known in the art, and antisense oligonucleotides or larger fragments can be designed from various locations along the coding or control regions of sequences encoding LIPAM (Agrawal, S., ed. (1996) Antisense Therapeutics, Humana Press, Totawa N.J.).

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein (Slater, J. E. et al. (1998) J. Allergy Clin. Immunol. 102:469-475; Scanlon, K. J. et al. (1995) FASEB J. 9:1288-1296). Antisense sequences can also be introduced intracellularly through the use of viral vectors, such as retrovirus and adeno-associated virus vectors (Miller, A. D. (1990) Blood 76:271-278; Ausubel et al., supra; Uckert, W. and W. Walther (1994) Pharmacol. Ther. 63:323-347). Other gene delivery mechanisms include liposome-derived systems, artificial viral envelopes, and other systems known in the art (Rossi, J. J. (1995) Br. Med. Bull. 51:217-225; Boado, R. J. et al. (1998) J. Pharm. Sci. 87:1308-1315; Morris, M. C. et al. (1997) Nucleic Acids Res. 25:2730-2736).

In another embodiment of the invention, polynucleotides encoding LIPAM may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in LIPAM expression or regulation causes disease, the expression of LIPAM from an appropriate population of transduced cells may alleviate the clinical manifestations caused by the genetic deficiency.

In a further embodiment of the invention, diseases or disorders caused by deficiencies in LIPAM are treated by constructing mammalian expression vectors encoding LIPAM and introducing these vectors by mechanical means into LIPAM-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J.-L. and H. Récipon (1998) Curr. Opin. Biotechnol. 9:445-450).

Expression vectors that may be effective for the expression of LIPAM include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PThT-ON, PTRE2, PTRE2-LUC, PTK-HYG (BD Clontech, Palo Alto Calif.). LIPAM may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding LIPAM from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to LIPAM expression are treated by constructing a retrovirus vector consisting of (i) the polynucleotide encoding LIPAM under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA.92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding LIPAM to cells which have one or more genetic abnormalities with respect to the expression of LIPAM. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999; Annu. Rev. Nutr. 19:511-544) and Verma, I. M. and N. Soria (1997; Nature 18:389:239-242).

In another embodiment, a herpes-based, gene therapy delivery system is used to deliver polynucleotides encoding LIPAM to target cells which have one or more genetic abnormalities with respect to the expression of LIPAM. The use of herpes simplex virus (HSV)-based vectors may be especially valuable for introducing LIPAM to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999; J. Virol. 73:519-532) and Xu, H. et al. (1994; Dev. Biol. 163:152-161). The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding LIPAM to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for LIPAM into the alphavirus genome in place of the capsid-coding region results in the production of a large number of LIPAM-coding RNAs and the synthesis of high levels of LIPAM in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of LIPAM into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) in Huber, B. E. and B. I. Carr, Molecular and Immunologic Approaches, Futura Publishing, Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding LIPAM.

Specific ribozyme cleavage sites within any potential RNA target are initially identified by scanning the target-molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA molecules encoding LIPAM. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytosine, guanine, thymine, and uracil which are not as easily recognized by endogenous endonucleases.

In other embodiments of the invention, the expression of one or more selected polynucleotides of the present invention can be altered, inhibited, decreased, or silenced using RNA interference (RNAi) or post-transcriptional gene silencing (PTGS) methods known in the art. RNAi is a post-transcriptional mode of gene silencing in which double-stranded RNA (dsRNA) introduced into a targeted cell specifically suppresses the expression of the homologous gene (i.e., the gene bearing the sequence complementary to the dsRNA). This effectively knocks out or substantially reduces the expression of the targeted gene. PTGS can also be accomplished by use of DNA or DNA fragments as well. RNAi methods are described by Fire, A. et al. (1998; Nature 391:806-811) and Gura, T. (2000; Nature 404:804-808). PTGS can also be initiated by introduction of a complementary segment of DNA into the selected tissue using gene delivery and/or viral vector delivery methods described herein or known in the art.

RNAi can be induced in mammalian cells by the use of small interfering RNA also known as siRNA. siRNA are shorter segments of dsRNA (typically about 21 to 23 nucleotides in length) that result in vivo from cleavage of introduced dsRNA by the action of an endogenous ribonuclease. siRNA appear to be the mediators of the RNAi effect in mammals. The most effective siRNAs appear to be 21 nucleotide dsRNAs with 2 nucleotide 3′ overhangs. The use of siRNA for inducing RNAi in mammalian cells is described by Elbashir, S. M. et al. (2001; Nature 411:494-498).

siRNA can be generated indirectly by introduction of dsRNA into the targeted cell. Alternatively, siRNA can be synthesized directly and introduced into a cell by transfection methods and agents described herein or known in the art (such as liposome-mediated transfection, viral vector methods, or other polynucleotide delivery/introductory methods). Suitable siRNAs can be selected by examining a transcript of the target polynucleotide (e.g., mRNA) for nucleotide sequences downstream from the AUG start codon and recording the occurrence of each nucleotide and the 3′ adjacent 19 to 23 nucleotides as potential siRNA target sites, with sequences having a 21 nucleotide length being preferred. Regions to be avoided for target siRNA sites include the 5′ and 3′ untranslated regions (ULTRs) and regions near the start codon (within 75 bases), as these may be richer in regulatory protein binding sites. UTR-binding proteins and/or translation initiation complexes may interfere with binding of the siRNP endonuclease complex. The selected target sites for siRNA can then be compared to the appropriate genome database (e.g., human, etc.) using BLAST or other sequence comparison algorithms known in the art. Target sequences with significant homology to other coding sequences can be eliminated from consideration. The selected siRNAs can be produced by chemical synthesis methods known in the art or by in vitro transcription using commercially available methods and kits such as the SILENCER siRNA construction kit (Ambion, Austin Tex.).

In alternative embodiments, long-term gene silencing and/or RNAi effects can be induced in selected tissue using expression vectors that continuously express siRNA. This can be accomplished using expression vectors that are engineered to express hairpin RNAs (shRNAs) using methods known in the art (see, e.g., Brummelkamp, T. R. et al. (2002) Science 296:550-553; and Paddison, P. J. et al. (2002) Genes Dev. 16:948-958). In these and related embodiments, shRNAs can be delivered to target cells using expression vectors known in the art. An example of a suitable expression vector for delivery of siRNA is the PSILENCER1.0-U6 (circular) plasmid (Ambion). Once delivered to the target tissue, shRNAs are processed in vivo into siRNA-like molecules capable of carrying out gene-specific silencing.

In various embodiments, the expression levels of genes targeted by RNAi or PTGS methods can be determined by assays for mRNA and/or protein analysis. Expression levels of the mRNA of a targeted gene can be determined, for example, by northern analysis methods using the NORTHERNMAX-GLY kit (Ambion); by microarray methods; by PCR methods; by real time PCR methods; and by other RNA/polynucleotide assays known in the art or described herein. Expression levels of the protein encoded by the targeted gene can be determined, for example, by microarray methods; by polyacrylamide gel electrophoresis; and by Western analysis using standard techniques known in the art.

An additional embodiment of the invention encompasses a method for screening for a compound which is effective in altering expression of a polynucleotide encoding LIPAM. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased LIPAM expression or activity, a compound which specifically inhibits expression of the polynucleotide encoding LIPAM may be therapeutically useful, and in the treatment of disorders associated with decreased LIPAM expression or activity, a compound which specifically promotes expression of the polynucleotide encoding LIPAM may be therapeutically useful.

In various embodiments, one or more test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding LIPAM is exposed to at least one test compound thus obtained. The sample may comprise, for example, an intact or permeabilized cell, or an in vitro cell-free or reconstituted biochemical system. Alterations in the expression of a polynucleotide encoding LIPAM are assayed by any method commonly known in the art. Typically, the expression of a specific nucleotide is detected by hybridization with a probe having a nucleotide sequence complementary to the sequence of the polynucleotide encoding LIPAM. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art (Goldman, C. K. et al. (1997) Nat. Biotechnol. 15:462-466).

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of LIPAM, antibodies to LIPAM, and mimetics, agonists, antagonists, or inhibitors of LIPAM.

In various embodiments, the compositions described herein, such as pharmaceutical compositions, may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood; circulation (see, e.g., Patton, J. S. et al., U.S. Pat. No. 5,997,848). Pulmonary delivery allows administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising LIPAM or fragments thereof. For example, liposome preparations containing a cell-impermeable macromolecule may promote cell fusion and intracellular delivery of the macromolecule. Alternatively, LIPAM or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example LIPAM or fragments thereof, antibodies of LIPAM, and agonists, antagonists or inhibitors of LIPAM, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 Mg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind LIPAM may be used for the diagnosis of disorders characterized by expression of LIPAM, or in assays to monitor patients being treated with LIPAM or agonists, antagonists, or inhibitors of LIPAM. Antibodies useful for diagnostic purposes may be prepared in the same manner as described above for therapeutics. Diagnostic assays for LIPAM include methods which utilize the antibody and a label to detect LIPAM in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring LIPAM, including ELISAs, RIAs, and FACS, are known in the art and provide a basis for diagnosing altered or abnormal levels of LIPAM expression. Normal or standard values for LIPAM expression are established by combining body fluids or cell extracts taken from normal mammalian subjects, for example, human subjects, with antibodies to LIPAM under conditions suitable for complex formation. The amount of standard complex formation may be quantitated by various methods, such as photometric means. Quantities of LIPAM expressed in subject, control, and disease samples from biopsied tissues are compared with the standard values. Deviation between standard and subject values establishes the parameters for diagnosing disease.

In another embodiment of the invention, polynucleotides encoding LIPAM may be used for diagnostic purposes. The polynucleotides which may be used include oligonucleotides, complementary RNA and DNA molecules, and PNAs. The polynucleotides may be used to detect and quantify gene expression in biopsied tissues in which expression of LIPAM may be correlated with disease. The diagnostic assay may be used to determine absence, presence, and excess expression of LIPAM, and to monitor regulation of LIPAM levels during therapeutic intervention.

In one aspect, hybridization with PCR probes which are capable of detecting polynucleotides, including genomic sequences, encoding LIPAM or closely related molecules may be used to identify nucleic acid sequences which encode LIPAM. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding LIPAM, allelic variants, or related sequences.

Probes may also be used for the detection of related sequences, and may have at least 50% sequence identity to any of the LIPAM encoding sequences. The hybridization probes of the subject invention may be DNA or RNA and may be derived from the sequence of SEQ ID NO:22-42 or from genomic sequences including promoters, enhancers, and introns of the LIPAM gene.

Means for producing specific hybridization probes for polynucleotides encoding LIPAM include the cloning of polynucleotides encoding LIPAM or LIPAM derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotides encoding LIPAM may be used for the diagnosis of disorders associated with expression of LIPAM. Examples of such disorders include, but are not limited to, a cancer, such as adenocarcinoma, leukemia, lymphoma, melanoma, myeloma, sarcoma, teratocarcinoma, and, in particular, cancers of the adrenal gland, bladder, bone, bone marrow, brain, breast, cervix, colon, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a cardiovascular disorder such as arteriovenous fistula, atherosclerosis, hypertension, vasculitis, Raynaud's disease, aneurysms, arterial dissections, varicose veins, thrombophlebitis and phlebothrombosis, vascular tumors, and complications of thrombolysis, balloon angioplasty, vascular replacement, and coronary artery bypass graft surgery, congestive heart failure, ischemic heart disease, angina pectoris, myocardial infarction, hypertensive heart disease, degenerative valvular heart disease, calcific aortic valve stenosis, congenitally bicuspid aortic valve, mitral annular calcification, mitral valve prolapse, rheumatic fever and rheumatic heart disease, infective endocarditis, nonbacterial thrombotic endocarditis, endocarditis of systemic lupus erythematosus, carcinoid heart disease, cardiomyopathy, myocarditis, pericarditis, neoplastic heart disease, congenital heart disease, and complications of cardiac transplantation, congenital lung anomalies, atelectasis, pulmonary congestion and edema, pulmonary embolism, pulmonary hemorrhage, pulmonary infarction, pulmonary hypertension, vascular sclerosis, obstructive pulmonary disease, restrictive pulmonary disease, chronic, obstructive pulmonary disease, emphysema, chronic bronchitis, bronchial asthma, bronchiectasis, bacterial pneumonia, viral and mycoplasmal pneumonia, lung abscess, pulmonary tuberculosis, diffuse interstitial diseases, pneumoconioses, sarcoidosis, idiopathic pulmonary fibrosis, desquamative interstitial pneumonitis, hypersensitivity pneumonitis, pulmonary eosinophilia bronchiolitis obliterans-organizing pneumonia, diffuse pulmonary hemorrhage syndromes, Goodpasture's syndromes, idiopathic pulmonary hemosiderosis, pulmonary involvement in collagen-vascular disorders, pulmonary alveolar proteinosis, lung tumors, inflammatory and noninflammatory pleural effusions, pneumothorax, pleural tumors, drug-induced lung disease, radiation-induced lung disease, and complications of lung transplantation; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a gastrointestinal disorder such as dysphagia, peptic esophagitis, esophageal spasm, esophageal stricture, esophageal carcinoma, dyspepsia, indigestion, gastritis, gastric carcinoma, anorexia, nausea, emesis, gastroparesis, antral or pyloric edema, abdominal angina, pyrosis, gastroenteritis, intestinal obstruction, infections of the intestinal tract, peptic ulcer, cholelithiasis, cholecystitis, cholestasis, pancreatitis, pancreatic carcinoma, biliary tract disease, hepatitis, hyperbilirubinemia, cirrhosis, passive congestion of the liver, hepatoma, infectious colitis, ulcerative colitis, ulcerative proctitis, Crohn's disease, Whipple's disease, Mallory-Weiss syndrome, colonic carcinoma, colonic obstruction, irritable bowel syndrome, short bowel syndrome, diarrhea, constipation, gastrointestinal hemorrhage, acquired immunodeficiency syndrome (AIDS) enteropathy, jaundice, hepatic encephalopathy, hepatorenal syndrome, hepatic steatosis, hemochromatosis, Wilson's disease, alpha₁-antitrypsin deficiency, Reye's syndrome, primary sclerosing cholangitis, liver infarction, portal vein obstruction and thrombosis, centrilobular necrosis, peliosis hepatis, hepatic vein thrombosis, veno-occlusive disease, preeclampsia, eclampsia, acute fatty liver of pregnancy, intrahepatic cholestasis of pregnancy, and hepatic tumors including nodular hyperplasias, adenomas, and carcinomas; and a disorder of lipid metabolism such as fatty liver, cholestasis, primary biliary cirrhosis, carnitine deficiency, carnitine palmitoyltransferase deficiency, myoadenylate deaminase deficiency, hypertriglyceridemia, lipid storage disorders such Fabry's disease, Gaucher's disease, Niemann-Pick' s disease, metachromatic leukodystrophy, adrenoleukodystrophy, GM₂ gangliosidosis, and ceroid lipofuscinosis, abetalipoproteinemia, Tangier disease, hyperlipoproteinemia, diabetes mellitus, lipodystrophy, lipomatoses, acute panniculitis, disseminated fat necrosis, adiposis dolorosa, lipoid adrenal hyperplasia, minimal change disease, lipomas, atherosclerosis, hypercholesterolemia, hypercholesterolemia with hypertriglyceridemia, primary hypoalphalipoproteinemia, hypothyroidism, renal disease, liver disease, lecithin:cholesterol acyltransferase deficiency, cerebrotendinous xanthomatosis, sitosterolemia, hypocholesterolemia, Tay-Sachs disease, Sandhoff's disease, hyperlipidemia, hyperlipemia, lipid myopathies, and obesity. Polynucleotides encoding LIPAM may be used in Southern or northern analysis, dot blot, or other membrane-based technologies; in PCR technologies; in dipstick, pin, and multiformat ELISA-like assays; and in microarrays utilizing fluids or tissues from patients to detect altered LIPAM expression. Such qualitative or quantitative methods are well known in the art.

In a particular embodiment, polynucleotides encoding LIPAM may be used in assays that detect the presence of associated disorders, particularly those mentioned above. Polynucleotides complementary to sequences encoding LIPAM may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding LIPAM in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of LIPAM, a normal or standard profile for expression is established. This may be accomplished by combining body fluids or cell extracts taken from normal subjects, either animal or human, with a sequence, or a fragment thereof, encoding LIPAM, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding LIPAM may involve the use of PCR. These oligomers may be chemically synthesized, generated enzymatically, or produced in vitro. Oligomers will preferably contain a fragment of a polynucleotide encoding LIPAM, or a fragment of a polynucleotide complementary to the polynucleotide encoding LIPAM, and will be employed under optimized conditions for identification of a specific gene or condition. Oligomers may also be employed under less stringent conditions for detection or quantification of closely related DNA or RNA sequences.

In a particular aspect, oligonucleotide primers derived from polynucleotides encoding LIPAM may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from polynucleotides encoding LIPAM are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations (Taylor, J. G. et al. (2001) Trends Mol. Med. 7:507-512; Kwok, P.-Y. and Z. Gu (1999) Mol. Med. Today 5:538-543; Nowotny, P: et al. (2001) Curr. Opin. Neurobiol. 11:637-641).

Methods which may also be used to quantify the expression of LIPAM include radiolabeling or biotinylating nucleotides, coamplification of a control nucleic acid, and interpolating results from standard curves (Melby, P. C. et al. (1993) J. Immunol. Methods 159:235-244; Duplaa, C. et al. (1993) Anal. Biochem. 212:229-236). The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotides described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, LIPAM, fragments of LIPAM, or antibodies specific for LIPAM may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time (Seilhamer et al., “Comparative Gene Transcript Analysis,” U.S. Pat. No. 5,840,484; hereby expressly incorporated by reference herein). Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript-images which profile the expression of the polynucleotides of the present invention may also be used in-conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity (see, for example, Press Release 00-02 from the National Institute of Environmental Health Sciences, released Feb. 29, 2000, available at niehs.nih.gov/oc/news/toxchip.htm). Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by-sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional tot the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data may be obtained for definitive protein identification.

A proteomic profile may also be generated using antibodies specific for LIPAM to quantify the levels of LIPAM expression. In one embodiment, the antibodies are used as elements on a microarray, and protein expression levels are quantified by contacting the microarray with the sample and detecting the levels of protein bound to each array element (Lueking, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art (Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/25116; Shalon, D. et al. (1995) PCT application WO95/35505; Heller, R. A. et al. (1997) Proc. Natl. Acad. Sci. USA 94:2150-2155; Heller, M. J. et al. (1997) U.S. Pat. No. 5,605,662). Various types of microarrays are well known and thoroughly described in Schena, M., ed. (1999; DNA Microarrays: A Practical Approach, Oxford University Press, London).

In another embodiment of the invention, nucleic acid sequences encoding LIPAM may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (Harrington, J. J. et al. (1997) Nat. Genet. 15:345-355; Price, C. M. (1993) Blood Rev. 7:127-134; Trask, B. J. (1991) Trends Genet. 7:149-154). Once mapped, the nucleic acid sequences may be used to develop genetic linkage maps, for example, which correlate the inheritance of a disease state with the inheritance of a particular chromosome region or restriction fragment length polymorphism (RFLP) (Lander, E. S. and D. Botstein (1986) Proc. Natl. Acad. Sci. USA 83:7353-7357).

Fluorescent in situ hybridization (FISH) may be correlated with other physical and genetic map data (Heinz-Urhich, et al. (1995) in Meyers, supra, pp. 965-968). Examples of genetic map data can be found in various scientific journals or at the Online Mendelian Inheritance in Man (OIMN) World Wide Web site. Correlation between the location of the gene encoding LIPAM on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation (Gatti, R. A. et al. (1988) Nature 336:577-580). The nucleotide sequence of the instant invention may also be used to detect differences in the chromosomal location due to translocation, inversion, etc., among normal, carrier, or affected individuals.

In another embodiment of the invention, LIPAM, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between LIPAM and the agent being tested may be measured.

Another technique for drug screening provides for high throughput screening of compounds having suitable binding affinity to the protein of interest (Geysen, et al. (1984) PCT application WO84/03564). In this method, large numbers of different small test compounds are synthesized on a solid substrate. The test compounds are reacted with LIPAM, or fragments thereof, and washed. Bound LIPAM is then detected by methods well known in the art. Purified LIPAM can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding LIPAM specifically compete with a test compound for binding LIPAM. In this manner, antibodies can be used to detect the presence of any peptide which shares one or more antigenic determinants with LIPAM.

In additional embodiments, the nucleotide sequences which encode LIPAM may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any, way whatsoever.

The disclosures of all patents, applications, and publications mentioned above and below, including U.S. Ser. No. 60/426,105, U.S. Ser. No. 60/433,215, U.S. Ser. No. 60/453,127, U.S. Ser. No. 60/454,801, U.S. Ser. No. 60/465,619, U.S. Ser. No. 60/465,495, and U.S. Ser. No. 60/491,800 are hereby expressly incorporated by reference.

### EXAMPLES

I. Construction of cDNA Libraries

Incyte cDNAs are derived from cDNA libraries described in the LIFESEQ database (Incyte, Palo Alto Calif.). Some tissues are homogenized and lysed in guanidinium isothiocyanate, while others are homogenized and lysed in phenol or in a suitable mixture of denaturants, such as TRIZOL (Invitrogen), a monophasic solution of phenol and guanidine isothiocyanate. The resulting lysates are centrifuged over CsCl cushions or extracted with chloroform. RNA is precipitated from the lysates with either isopropanol or sodium acetate and ethanol, or by other routine methods.

Phenol extraction and precipitation of RNA are repeated as necessary to increase RNA purity. In some cases, RNA is treated with DNase. For most libraries, poly(A)+ RNA is isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA is isolated directly from tissue lysates using other RNA isolation kits, e.g., the POLY(A)PURE mRNA purification kit (Ambion, Austin Tex.).

In some cases, Stratagene is provided with RNA and constructs the corresponding cDNA libraries. Otherwise, cDNA is synthesized and cDNA libraries are constructed with the UNIZAP vector system (Stratagene) or SUPERSCRIPT plasmid system (Invitrogen), using the recommended procedures or similar methods known in the art (Ausubel et al., supra, ch. 5). Reverse transcription is initiated using oligo d(T) or random primers. Synthetic oligonucleotide adapters are ligated to double stranded cDNA, and the cDNA is digested with the appropriate restriction enzyme or enzymes. For most libraries, the cDNA is size-selected (300-1000 bp) using SEPHACRYL S1000, SEPHAROSE CL2B, or SEPHAROSE CL4B column chromatography (Amersham Biosciences) or preparative agarose gel electrophoresis. cDNAs are ligated into compatible restriction enzyme sites of the polylinker of a suitable plasmid, e.g., PBLUESCRIPT plasmid (Stratagene), PSPORT1 plasmid (Invitrogen, Carlsbad Calif.), PCDNA2.1 plasmid (Invitrogen), PBK-CMV plasmid (Stratagene), PCR2-TOPOTA plasmid (Invitrogen), PCMV-ICIS plasmid (Stratagene), pIGEN (Incyte, Palo Alto Calif.), pRARE (Incyte), or pINCY (Incyte), or derivatives thereof. Recombinant plasmids are transformed into competent E. coli cells including XL1-Blue, XL1-BlueMRF, or SOLR from Stratagene or DH5α, DH10B, or ElectroMAX DH10B from Invitrogen.

II. Isolation of cDNA Clones

Plasmids obtained as described in Example I are recovered from host cells by in vivo excision using the UNIAP vector system (Stratagene) or by cell lysis. Plasmids are purified using at least one of the following: a Magic or WIARD Minipreps DNA purification system (Promega); an AGTC-Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids are resuspended in 0.1 ml of distilled water and stored, with or without lyophilization, at 4° C.

Alternatively, plasmid DNA is amplified from host cell lysates using direct link PCR in a high-throughput format (Rao, V. B. (1994) Anal. Biochem. 216:1-14). Host cell lysis and thermal cycling steps are carried out in a single reaction mixture. Samples are processed and stored in 384-well plates, and the concentration of amplified plasmid DNA is quantified fluorometrically using PICOGREEN dye (Molecular Probes, Eugene, Oreg.) and a FLUOROSKAN II fluorescence scanner (Labsystems Oy, Helsinki, Finland).

III. Sequencing and Analysis

Incyte cDNA recovered in plasmids as described in Example II are sequenced as follows. Sequencing reactions are processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions are prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides are carried out using the MEGABACE 1000 DNA sequencing system (Amersham Biosciences); the ABI PRISM 373 or 377 sequencing system (Applied Biosystems) in conjunction with standard ABI protocols and base calling software; or other sequence analysis systems known in the art. Reading frames within the cDNA sequences are identified using standard methods (Ausubel et al., supra, ch. 7). Some of the cDNA sequences are selected for extension using the techniques disclosed in Example VIII.

Polynucleotide sequences derived from Incyte cDNAs are validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof are then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhabditis elegans, Saccharomyces cerevisiae, Schizosaccharomyces pombe, and Candida albicans (Incyte, Palo Alto Calif.); hidden Markov model (HMM)-based protein family databases such as PFAM, INCY, and TIGRFAM (Haft, D. H. et al. (2001) Nucleic Acids Res. 29:41-43); and HMM-based protein domain databases such as SMART (Schultz, J. et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families; see, for example, Eddy, S. R. (1996) Curr. Opin. Struct. Biol. 6:361-365.) The queries are performed using programs based on BLAST, FASTA, BLIMPS, and HMMER. The Incyte cDNA sequences are assembled to produce full length polynucleotide sequences. Alternatively, GenBank cDNAs, GenBank ESTs, stitched sequences, stretched sequences, or Genscan-predicted coding sequences (see Examples IV and V) are used to extend Incyte cDNA assemblages to full length. Assembly is performed using programs based on Phred, Phrap, and Consed, and cDNA assemblages are screened for open reading frames using programs based on GeneMark, BLAST, and FASTA. The full length polynucleotide sequences are translated to derive the corresponding full length polypeptide sequences. Alternatively, a polypeptide may begin at any of the methionine residues of the full length translated polypeptide. Full length polypeptide sequences are subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PEAM, INCY, and TIGRFAM; and HMM-based protein domain databases such as SMART. Full length polynucleotide sequences are also analyzed using MACDNASIS PRO software (MiraiBio, Alameda Calif.) and LASERGENE software (DNASTAR). Polynucleotide and polypeptide sequence alignments are generated using default parameters specified by the CLUSTAL algorithm as incorporated into the MEGALIGN multisequence alignment program (INASTAR), which also calculates the percent identity between aligned sequences.

Table 7 summarizes tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences are also used to identify polynucleotide sequence fragments from SEQ ID NO:22-42. Fragments from about 20 to about 4000 nucleotides which are useful in hybridization and amplification technologies are described in Table 4, column 2.

IV. Identification and Editing of Coding Sequences from Genomic DNA

Putative lipid-associated molecules are initially identified by running the Genscan gene identification program against public genomic sequence databases (e.g., gbpri and gbhtg). Genscan is a general-purpose gene identification program which analyzes genomic DNA sequences from a variety of organisms (Burge, C. and S. Karlin (1997) J. Mol. Biol. 268:78-94; Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once is set to 30 kb. To determine which of these Genscan predicted cDNA sequences encode lipid-associated molecules, the encoded polypeptides are analyzed by querying against PFAM models for lipid-associated molecules. Potential lipid-associated molecules are also identified by homology to Incyte cDNA sequences that have been annotated as lipid-associated molecules. These selected Genscan-predicted sequences are then compared by BLAST analysis to the genpept and gbpri public databases. Where necessary, the Genscan-predicted sequences are then edited by comparison to the top BLAST hit from genpept to correct errors in the sequence predicted by Genscan, such as extra or omitted exons. BLAST analysis is also used to find any Incyte cDNA or public cDNA coverage of the Genscan-predicted sequences, thus providing evidence for transcription. When Incyte cDNA coverage is available, this information is used to correct or confirm the Genscan predicted sequence. Full length polynucleotide sequences are obtained by assembling Genscan-predicted coding sequences with Incyte cDNA sequences and/or public cDNA sequences using the assembly process described in Example III. Alternatively, fall length polynucleotide sequences are derived entirely from edited or unedited Genscan-predicted coding sequences.

V. Assembly of Genomic Sequence Data with cDNA Sequence Data

“Stitched” Sequences

Partial cDNA sequences are extended with exons predicted by the Genscan gene identification program described in Example IV. Partial cDNAs assembled as described in Example III are mapped to genomic DNA and parsed into clusters containing related cDNAs and Genscan exon predictions from one or more genomic sequences. Each cluster is analyzed using an algorithm based on graph theory and dynamic programming to integrate cDNA and genomic information, generating possible splice variants that are subsequently confirmed, edited, or extended to create a full length sequence. Sequence intervals in which the entire length of the interval is present on more than one sequence in the cluster are identified, and intervals thus identified are considered to be equivalent by transitivity. For example, if an interval is present on a cDNA and two genomic sequences, then all three intervals are considered to be equivalent. This process allows unrelated but consecutive genomic sequences to be brought together, bridged by cDNA sequence. Intervals thus identified are then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) are given preference over linkages which change parent type (cDNA to genomic sequence). The resultant stitched sequences are translated and compared by BLAST analysis to the genpept and gbpri public databases. Incorrect exons predicted by Genscan are corrected by comparison to the top BLAST hit from genpept. Sequences are further extended with additional cDNA sequences, or by inspection of genomic DNA, when necessary.

“Stretched” Sequences

Partial DNA sequences are extended to full length with an algorithm based on BLAST analysis. First, partial cDNAs assembled as described in Example m are queried against public databases such as the GenBank primate, rodent, mammalian, vertebrate, and eukaryote databases using the BLAST program. The nearest GenBank protein homolog is then compared by BLAST analysis to either Incyte cDNA sequences or GenScan exon predicted sequences described in Example IV. A chimeric protein is generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both are used as probes to search for homologous genomic sequences from the public human genome databases. Partial DNA sequences are therefore “stretched” or extended by the addition of homologous genomic sequences. The resultant stretched sequences are examined to determine whether they contain a complete gene.

VI. Chromosomal Mapping of LIPAM Encoding Polynucleotides

The sequences used to assemble SEQ ID NO:22-42 are compared with sequences from the Incyte LIFESEQ database and public domain databases using BLAST and other implementations of the Smith-Waterman algorithm. Sequences from these databases that matched SEQ ID NO:22-42 are assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon are used to determine if any of the clustered sequences have been previously mapped. Inclusion of a mapped sequence in a cluster results in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Genethon which provide boundaries for radiation hybrid markers whose sequences were included in each of the clusters. Human genome maps and other resources available to the public, such as the NCBI “GeneMap'99” World Wide Web site (ncbi.nlm.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound (Sambrook and Russell, supra, ch. 7; Ausubel et al., supra, ch. 4).

Analogous computer techniques applying BLAST are used to search for identical or related molecules in databases such as GenBank or LIFESEQ (Incyte). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP); and 4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding LIPAM are analyzed with respect to the tissue sources from which they are derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding LIPAM. cDNA sequences and cDNA library/tissue information are found in the LIFESEQ database (Incyte, Palo Alto Calif.).

VIII. Extension of LIPAM Encoding Polynucleotides

Full length polynucleotides are produced by extension of an appropriate fragment of the full length molecule using oligonucleotide primers designed from this fragment. One primer is synthesized to initiate 5′ extension of the known fragment, and the other primer is synthesized to initiate 3′ extension of the known fragment. The initial primers are designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations is avoided.

Selected human cDNA libraries are used to extend the sequence. If more than one extension is necessary or desired, additional or nested sets of primers are designed.

High fidelity amplification is obtained by PCR using methods well known in the art. PCR is performed in 96-well-plates using the PTC-200 thermal cycler (MJ Research, Inc.). The reaction mix contains DNA template, 200 nmol of each primer, reaction buffer containing Mg²⁺, (NH₄)₂SO₄, and 2-mercaptoethanol, Taq DNA polymerase (Amersham Biosciences), ELONGASE enzyme (Invitrogen), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ are as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: 'storage at 4° C.

The concentration of DNA in each well is determined by dispensing 100 μl PICOGREEN quantitation reagent (0.25% (v/v) PICOGREEN; Molecular Probes, Eugene Oreg.) dissolved in 1× TE and 0.5 μl of undiluted PCR product into each well of an opaque fluorimeter plate (Corning Costar, Acton Mass.), allowing the DNA to bind to the reagent. The plate is scanned in a Fluoroskan II (Labsystems Oy, Helsinki, Finland) to measure the fluorescence of the sample and to quantify the concentration of DNA. A 5 μl to 10 μl aliquot of the reaction mixture is analyzed by electrophoresis on a 1% agarose gel to determine which reactions are successful in extending the sequence.

The extended nucleotides are desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides are separated on low concentration (0.6 to 0.8%) agarose gels, fragments are excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells are selected on antibiotic-containing media, and individual colonies are picked and cultured overnight at 37° C. in 384-well plates in LB/2× carb liquid media.

The cells are lysed, and DNA is amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA is quantified by PICOGREEN reagent (Molecular Probes) as described above. Samples with low DNA recoveries are reamplified using the same conditions as described above. Samples are diluted with 20% dimethysulfoxide (1:2, v/v), and sequenced using DYENAMIC energy transfer sequencing primers and the DYENAMIC DIRECT kit (Amersham Biosciences) or the ABI PRISM BIGDYE Terminator cycle sequencing ready reaction kit (Applied Biosystems).

In like manner, full length polynucleotides are verified using the above procedure or are used to obtain 5′ regulatory sequences using the above procedure along with oligonucleotides designed for such extension, and an appropriate genomic library.

IX. Identification of Single Nucleotide Polymorphisms in LIPAM Encoding Polynucleotides

Common DNA sequence variants known as single nucleotide polymorphisms (SNPs) are identified in SEQ ID NO:22-42 using the LIESEQ database (Incyte). Sequences from the same gene are clustered together and assembled as described in Example III, allowing the identification of all sequence variants in the gene. An algorithm consisting of a series of filters is used to distinguish SNPs from other sequence variants. Preliminary filters remove the majority of basecall errors by requiring a minimum Phred quality score of 15, and remove sequence alignment errors and errors resulting from improper trimming of vector sequences, chimeras, and splice variants. An automated procedure of advanced chromosome analysis is applied to the original chromatogram files in the vicinity of the putative SNP. Clone error filters use statistically generated algorithms to identify errors introduced during laboratory processing, such as those caused by reverse transcriptase, polymerase, or somatic mutation. Clustering error filters use statistically generated algorithms to identify errors resulting from clustering of close homologs or pseudogenes, or due to contamination by non-human sequences. A final set of filters removes duplicates and SNPs found in immunoglobulins or T-cell receptors.

Certain SNPs are selected for further characterization by mass spectrometry using the high throughput MASSARRAY system (Sequenom, Inc.) to analyze allele frequencies at the SNP sites in four different human populations. The Caucasian population comprises 92 individuals (46 male, 46 female), including 83 from Utah, four French, three Venezualan, and two Amish individuals. The African population comprises 194 individuals (97 male, 97 female), all African Americans. The Hispanic population comprises 324 individuals (162 male, 162 female), all Mexican Hispanic. The Asian population comprises 126 individuals (64 male, 62 female) with a reported parental breakdown of 43% Chinese, 31% Japanese, 13% Korean, 5% Vietnamese, and 8% other Asian. Allele frequencies are first analyzed in the Caucasian population; in some cases those SNPs which show no allelic variance in this population are not further tested in the other three populations.

X. Labeling and use of Individual Hybridization Probes

Hybridization probes derived from SEQ ID NO:22-42 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Biosciences), and T4 polynucleotide kinase (DuPont NEN, Boston Mass.). The labeled oligonucleotides are substantially purified using a SEPHADEX G-25 superfine size exclusion dextran bead column (Amersham Biosciences). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to NYTRAN PLUS nylon membranes (Schleicher & Schuell, Durham N. H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (ink-jet printing; see, e.g., Baldeschweiler et al., supra), mechanical microspotting technologies, and derivatives thereof. The substrate in each of the aforementioned technologies should be uniform and solid with a non-porous surface (Schena, M., ed. (1999) DNA Microarrays: A Practical Approach, Oxford University Press, London). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements (Schena, M. et al. (1995) Science 270:467-470; Shalon, D. et al. (1996) Genome Res. 6:639-645; Marshall, A. and J. Hodgson (1998) Nat. Biotechnol. 16:27-31).

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 pg/μl oligo-(dT) primer (21mer), 1× first strand buffer, 0.03 units/μl RNase inhibitor, 500 μM DATP, 500 μM dGTP, 500 μM dTTP, 40 μM dCTP, 40 μM dCTP-Cy3 (BDS) or dCTP-Cy5 (Amersham Biosciences). The reverse transcription reaction is performed in a 25 ml volume containing 200 ng poly(A)⁺ RNA with GEMBRIGHT kits (Incyte). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (BD Clontech, Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma-Aldrich, St. Louis Mo.) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample, mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μL of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that, location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte). Array elements that exhibit at least about a two-fold change in expression, a signal-to-background ratio of at least about 2.5, and an element spot size of at least about 40%, are considered to be differentially expressed.

Expression

SEQ ID NO:22 and SEQ ID NO:24 showed differential expression, as determined by microarray analysis. Expression of SEQ ID NO:22 was increased by at least two-fold in lung squamous cell carcinoma versus uninvolved lung tissue from the same donor. Expression of SEQ ID NO:24 was decreased by at least two-fold in lung tumor tissue versus normal lung tissue from the same donor for six out of ten donors. Therefore, in various embodiments, SEQ ID NO:22 and SEQ ID NO:24 can be used for one or more of the following: i) monitoring treatment of lung cancer, ii) diagnostic assays for lung cancer, and iii) developing therapeutics and/or other treatments for lung cancer.

The expression of SEQ ID NO:24 was up-regulated in a prostate carcinoma cell line isolated from a metastatic site in the brain versus primary prostate epithelial cells isolated from a normal donor. Primary prostate epithelial cells were compared with prostate carcinomas representative of the different stages of tumor progression. Cell lines compared included: a) PrEC, a primary prostate epithelial cell line isolated from a normal donor, b) DU 145, a prostate carcinoma cell line isolated from a metastatic sitei in the brain of 69-year old male with widespread metastatic prostate carcinoma, c) LNCAP, a prostate carcinoma cell line isolated from a lymph node biopsy of a 50-year-old male with metastatic prostate carcinoma, and d) PC-3, a prostate adenocarcinoma cell line isolated from a metastatic site in the bone of a 62-year-old male with grade IV prostate adenocarcinoma. Cells grown under restrictive conditions were compared to normal PrECs grown under restrictive conditions; cells were grown in basal media in the absence of growth factors and hormones. Expression of SEQ ID NO:24 was decreased by at least two-fold in DU 145 cells as compared to the non-malignant PrEC cells. Therefore, in various embodiments, SEQ ID NO:24 can be used for one or more of the following: i) monitoring treatment of prostate cancer, ii) diagnostic assays for prostate cancer, and iii) developing therapeutics and/or other treatments for prostate cancer.

In another example, expression of SEQ ID NO:29 was upregulated in diseased colon tissue versus normal colon tissue as determined by microarray analysis. Matched normal and tumor samples from a 58-year-old female diagnosed with mucinous adenocarcinoma (Huntsman Cancer Institute, Salt Lake City, Utah) were compared by competitive hybridization. Expression of SEQ ID NO:29 was increased at least two-fold in colon adenocarcinoma tissue when compared to normal colon tissue from the same donor. Therefore, in various embodiments, SEQ ID NO:29 can be used for one or more of the following: i) monitoring treatment of colon cancer, ii) diagnostic assays for colon cancer, and iii) developing therapeutics and/or other treatments for colon cancer.

To evaluate the variation in gene expression in peripheral blood mononuclear cells (PBMCs) from healthy volunteer donors in response to SEB, PBMCs from healthy donors were compared to untreated PBMCs from the same donor. Cells were activated with 1 ng/ml SEB; treated PBMCs were compared to matching PBMCs kept in culture in the presence of medium alone. The expression of SEQ ID NO:33 was increased by at least two-fold in the PBMCs exposed to SEB for 72 hours. Therefore, in various embodiments, SEQ ID NO:33 can be used for one or more of the following: i) monitoring treatment of immune disorders and related diseases and conditions, ii) diagnostic assays for immune disorders and related diseases and conditions, and iii) developing therapeutics and/or other treatments for immune disorders and related diseases and conditions.

SEQ ID NO:38 showed differential expression in prostate cancer cell lines, as determined by microarray analysis. Primary prostate epithelial cells were compared with prostate carcinomas representative of the different stages of tumor progression. Cell lines compared included: a) PrEC, a primary prostate epithelial cell line isolated from a normal donor, b) DU 145, a prostate carcinoma cell line isolated from a metastatic site in the brain of 69-year old male with widespread metastatic prostate carcinoma, c) LNCaP, a prostate carcinoma cell line isolated from a lymph node biopsy of a 50-year-old male with metastatic prostate carcinoma, and d) PC-3, a prostate adenocarcinoma cell line isolated from a metastatic site in the bone of a 62-year-old male with grade IV prostate adenocarcinoma. In one set of experiments, all cell lines were grown in basal media in the absence of growth factors and hormones. In another set of experiments, all cell lines were grown under optimal growth conditions, in the presence of growth factors and nutrients. SEQ ID NO:38 expression was increased at least 2-fold in DU 145 cells, when compared to expression levels detected in PrEC cells, in both sets of experiments. Therefore, in various embodiments, SEQ ID NO:38 can be used for one or more of the following: i) monitoring treatment of prostate cancer, ii) diagnostic assays for prostate cancer, and iii) developing therapeutics and/or other treatments for prostate cancer. 1520

In an alternative example, SEQ ID NO:40 was overexpressed by at least two fold in matched tumorous versus normal colon tissues in two out of seven donors tested. Therefore, in various embodiments, SEQ ID NO:40 can be used for one or more of the following: i) monitoring treatment of colon cancer, ii) diagnostic assays for colon cancer, and iii) developing therapeutics and/or other treatments for colon cancer.

In an alternative example, SEQ ID NO:40 was downregulated by at least two fold in matched tumorous versus normal lung tissues in the one donor tested. Therefore, in various embodiments, SEQ ID NO:40 can be used for one or more of the following: i) monitoring treatment of lung cancer, ii) diagnostic assays for lung cancer, and iii) developing therapeutics and/or other treatments for lung cancer.

In an alternative example, SEQ ID NO:40 was downregulated by at least two fold in matched tumorous versus normal ovarian tissues in the one donor tested. This result held true when the experiment was repeated. Therefore, in various embodiments, SEQ ID NO:40 can be used for one or more of the following: i) monitoring treatment of ovarian cancer, ii) diagnostic assays for ovarian cancer, and iii) developing therapeutics and/or other treatments for ovarian cancer.

In an alternative example, the gene expression profile of a nonmalignant mammary epithelial cell line was compared to the gene expression profiles of breast carcinoma lines at different stages of tumor progression. HMEC is a primary breast epithelial cell line isolated from a normal donor. Cell lines compared included: a) MCF-10A, a breast mammary gland (luminal ductal characteristics) cell line isolated from a 36-year-old woman with fibrocystic breast disease, b) MCF7, a nonmalignant breast adenocarcinoma cell line isolated from the pleural effusion of a 69-year-old female, c) BT-20, a breast carcinoma cell line derived in vitro from the cells emigrating out of thin slices of tumor mass isolated from a 74-year-old female, d) T-47D, a breast carcinoma cell line isolated from a pleural effusion obtained from a 54-year-old female with an infiltrating ductal carcinoma of the breast, e) Sk-BR-3, a breast adenocarcinoma cell line isolated from a malignant pleural effusion of a 43-year-old female, f) MDA-mb-231, a breast tumor cell line isolated from the pleural effusion of a 51-year-old female, g) MDA-mb-435S, a spindle-shaped strain that evolved from the parent line (435) isolated by R. Cailleau from pleural effusion of a 31-year-old female with metastatic, ductal adenocarcinoma of the breast. SEQ ID NO:41 was found to be downregulated by at least two-fold in MCF7, MCF-10A, BT-20, T-47D, Sk-BR-3, MDA-mb-231, and MA-mb-435S cell lines. Therefore, in various embodiments, SEQ ID NO:41 can be used for one or more of the following: i) monitoring treatment of breast cancer, ii) diagnostic assays for breast cancer, and iii) developing therapeutics and/or other treatments for breast cancer.

In an alternative example, human aortic endothelial cells (HMVECdNeos) were grown to 85% confluency and then treated with 10 ng/ml TNF-α for 1, 2, 4, 8, and 24 hours. TNF-α-treated cells were compared to untreated HMVECdNeos collected at 85% confluency (0 hour). SEQ ID NO:41 was found to be upregulated in cells treated with TNF-α for 2, 4, 8, and 24 hours. Therefore, in various embodiments, SEQ ID NO:41 can be used for one or more of the following: i) monitoring treatment of immune disorders and related diseases and conditions, ii) diagnostic assays for immune disorders and related diseases and conditions, and iii) developing therapeutics and/or other treatments for immune disorders and related diseases and conditions.

In an alternative example, human umbilical vein endothelial cells (HUVECs) were grown to 85% confluency and then treated with 10 ng/ml TNF-α for 0.33, 0.66, 1, 4, 8, 24, 48, and 72 hours. TNF-α-treated cells were compared to untreated HUVECs collected at 85% confluency (0 hour). SEQ ID NO:41 was found to be upregulated in cells treated with TNF-α for 4, 8, 24, 48, and 72 hours. Therefore, in various embodiments, SEQ ID NO:41 can be used for one or more of the following: i) monitoring treatment of immune disorders and related diseases and conditions, ii) diagnostic assays for immune disorders and related diseases and conditions, and iii) developing therapeutics and/or other treatments for immune disorders and related diseases and conditions.

In an alternative example, SEQ ID NO:25, SEQ ID NO:26, SEQ ID NO:31, SEQ ID NO:32, and SEQ ID NO:41 showed tissue-specific expression as determined by microarray analysis. RNA samples isolated from a variety of normal human tissues were compared to a common reference sample. Tissues contributing to the reference sample were selected for their ability. to provide a complete distribution of RNA in the human body and include brain (4%), heart (7%), kidney (3%), lung (8%), placenta (46%), small intestine (9%), spleen (3%), stomach (6%), testis (9%), and uterus (5%). The normal tissues assayed were obtained from at least three different donors. RNA from each donor was separately isolated and individually hybridized to the microarray. Since these hybridization experiments were conducted using a common reference sample, differential expression values are directly comparable from one tissue to another. The expression of SEQ ID NO:25 and SEQ ID NO:26 was increased by at least two-fold in the thymus gland as compared to the reference sample. Therefore, SEQ ID NO:25 and SEQ ID NO:26 can be used as tissue markers for the thymus gland. The expression of SEQ ID NO:31 and SEQ ID NO:32 was increased by at least two-fold in jejunum as compared to the reference sample. Therefore, SEQ ID NO:31 and SEQ ID NO:32 can be used as a tissue marker for jejunum. The expression of SEQ ID NO:41 was increased by at least two-fold in thyroid gland as compared to the reference sample. Therefore, SEQ ID NO:41 can be used as a tissue marker for thyroid gland.

XII. Complementary Polynucleotides

Sequences complementary to the LIPAM-encoding sequences, or any parts thereof, are used to detect, decrease, or inhibit expression of naturally occurring LIPAM. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of LIPAM. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the LIPAM-encoding transcript.

XIII. Expression of LIPAM

Expression and purification of LIPAM is achieved using bacterial or virus-based expression systems. For expression of LIPAM in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express LIPAM upon induction with isopropyl beta-D-thiogalactopyranoside (IFTG). Expression of LIPAM in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding LIPAM by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus (Engelhard, E. K. et al. (1994) Proc. Natl. Acad. Sci. USA 91:3224-3227; Sandig, V. et al. (1996) Hum. Gene Ther. 7:1937-1945).

In most expression systems, LIPAM is synthesized, as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from LIPAM at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel et al. (supra, ch., 10 and 16). Purified LIPAM obtained by these methods can be used directly in the assays shown in Examples XVII and XVIII, where applicable.

XIV. Functional Assays

LIPAM function is assessed by expressing the sequences encoding LIPAM at physiologically elevated levels in mammalian cell culture systems. cDNA is subcloned into a mammalian expression vector containing a strong promoter that drives high levels of cDNA expression. Vectors of choice include PCMV SPORT plasmid (Invitrogen, Carlsbad Calif.) and PCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; BD Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994; Flow Cytometry, Oxford, New York N.Y.).

The influence of LIPAM on gene expression can be assessed using highly purified populations of cells transfected with sequences-encoding LIPAM and either CD64 or CD64-GFP. CD64 and CD64-GFP are expressed on the surface of transfected cells and bind to conserved regions of human immunoglobulin G (IgG). Transfected cells are efficiently separated from nontransfected cells using: magnetic beads coated with either human IgG or antibody against CD64 (DYNAL, Lake Success N.Y.). mRNA can be purified from the cells using methods well known by those of skill in the art. Expression of mRNA encoding LIPAM and other genes of interest can be analyzed by northern analysis or microarray techniques.

XV. Production of LIPAM Specific Antibodies

LIPAM substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the LIPAM amino acid sequence is analyzed using LASERGENE software, (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art (Ausubel et al., supra, ch. 11).

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity (Ausubel et al., supra). Rabbits are immunized with the oligopeptide-KLH complex in complete Freund's adjuvant. Resulting antisera are tested for antipeptide and anti-LIPAM activity by, for example, binding the peptide or LIPAM to a substrate, blocking with 1% BSA, reacting with rabbit antisera, washing, and reacting with radio-iodinated goat anti-rabbit IgG.

XVI. Purification of Naturally Occurring LIPAM Using Specific Antibodies

Naturally occurring or recombinant LIPAM is substantially purified by immunoaffinity chromatography using antibodies specific for LIPAM. An immunoaffinity column is constructed by covalently coupling anti-LIPAM antibody to an activated chromatographic resin, such as CNBr-activated SEPHAROSE (Amersham Biosciences). After the coupling, the resin is blocked and washed according to the manufacturer's instructions.

Media containing LIPAM are passed over the immunoaffinity column, and the column is washed under conditions that allow the preferential absorbance of LIPAM (e.g., high ionic strength buffers in the presence of detergent). The column is eluted under conditions that disrupt antibody/LIPAM binding (e.g., a buffer of pH 2 to pH 3, or a high concentration of a chaotrope, such as urea or thiocyanate ion), and LIPAM is collected.

XVII. Identification of Molecules which Interact with LIPAM

LIPAM, or biologically active fragments thereof, are labeled with ¹²⁵I Bolton-Hunter reagent (Bolton, A. E. and W. M. Hunter (1973) Biochem. J. 133:529-539). Candidate molecules previously arrayed in the wells of a multi-well plate are incubated with the labeled LIPAM, washed, and any wells with labeled LIPAM complex are assayed. Data obtained using different concentrations of LIPAM are used to calculate values for the number, affinity, and association of LIPAM with the candidate molecules.

Alternatively, molecules interacting with LIPAM are analyzed using the yeast two-hybrid system as described in Fields, S. and O. Song (1989; Nature 340:245-246), or using commercially available kits based on the two-hybrid system, such as the MATCHMAKER system (BD Clontech).

LIPAM may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVIII. Demonstration of LIPAM Activity

Selected candidate lipid molecules, such as C4 sterols, oxysterol, apolipoprotein E, and phospholipids, are arrayed in the wells of a multi-well plate. LIPAM, or biologically active fragments thereof, are labeled with ¹²⁵I Bolton-Hunter reagent. (See, e.g., Bolton A. E. and W. M. Hunter (1973) Biochem. J. 133:529-539.) The selected candidate lipid molecules are incubated with the labeled LIPAM and washed. Any wells with labeled LIPAM complex are assayed. Data obtained using different concentrations of LIPAM are used to calculate values for the number, affinity, and association of LIPAM with the candidate molecules. Significant binding of LIPAM to the candidate lipid molecules is indicative of LIPAM activity.

In the alternative, LIPAM activity is determined in a continuous fluorescent transfer assay using as substrate 1-palritoyl-2-pyrenyldecanoyl-phosphatidylinositol (Phy(10)PI). The assay measures the increase of pyrene monomer fluorescence intensity as a result of the transfer of pyrenylacyl (Pyr(x))-labeled phospholipid from quenched donor vesicles to unquenched acceptor vesicles (Van Paridon et al. (1988) Biochemistry 27:6208-6214). Donor vesicles consist of Pyr(x) phosphatidylinositol (Pyr(x)PI), 2,4,6-trinitrophenylphosphatidylethanolamine (TNP-PE) and egg phosphatidylcholine (PC) in a mol % ratio of 10:10:80 (2 nmol of total phospholipid). Acceptor vesicles consist of phosphatidic acid (PA) and egg PC in a mol % ratio of 5:95 (25-fold excess of total phospholipid). The reaction is carried out in 2 ml of 20 mM Tris-HCl, 5 mM EDTA, 200 mM NaCl (pH 7.4) containing 0.1 mg of BSA at 37° C. The reaction is initiated by the addition of 10-50 μl of LIPAM. Measurements are performed using a fluorimeter equipped with a thermostated cuvette holder and a stirring device. The initial slope of the progress curve is taken as an arbitrary unit of transfer activity (van Tiel, C. M. et al. (2000) J. Biol. Chem. 275:21532-21538; Westerman, J. et al. (1995) J. Biol. Chem. 270:14263-14266).

In the alternative, LIPAM activity is determined by measuring the rate of incorporation of a radioactive fatty acid precursor into fatty acyl-CoA. The final reaction contains 200 mM Tris-HCl, pH 7.5, 2.5 mM ATP, 8 mM MgCl₂, 2 mM EDTA, 20 mM NaF, 0.1% Triton X-100, 10 mM [³H]oleate, [³H]myristate or [¹⁴C]decanoate, 0.5 mM coenzyme A, and LIPAM in a total volume of 0.5 ml. The reaction is initiated with the addition of coenzyme A, incubated at 35° C. for 10 min, and terminated by the addition of 2.5 ml of isopropyl alcohol, n-heptane, 1 M H₂ SO₄ (40:10:1). Radioactive fatty acid is removed by organic extraction using n-heptane. Fatty acyl-CoA formed during the reaction remains in the aqueous fraction and is quantified by scintillation counting (Black, P. N. et al. (1997) J. Biol. Chem. 272:4896-4904).

In the alternative, LIPAM activity is determined by measuring the degradation of the sphingolipid glucosylceramide. 25-50 microunits glucocerebrosidase are incubated with varying concentrations of LIPAM in a 40 μl reaction at 37° C. for 20 min. The final reaction contains 50 mM sodium citrate pH 4.5, 20 ng human serum albumin, and 3.125 mM lipids in the form of liposomes, which contain lipids in the following proportions: [¹⁴C]glucosylceramide (3 mol %, 2.4 Ci/mol), cholesterol (23 mol %), phosphatidic acid (20 mol %), phosphatidylcholine (54 mol %). The reaction is stopped by the addition of 160 μl chloroform/methanol (2:1) and 20 Al 0.1% glucose, and shaking. After centrifugation at 4000 rpm, enzymatically released [¹⁴C]glucose in the aqueous phase is measured in a scintillation counter. LIPAM activity is determined by its effect on increasing the rate of glucosylceramide hydrolysis by glucocerebrosidase (Wilkening, G. et al. J. Biol. Chem. (1998) 273:30271-30278).

In the alternative, LIPAM activity can be demonstrated by an in vitro hydrolysis assay with vesicles containing 1-palmitoyl-2-[1-¹⁴C]oleoyl phosphatidylcholine (Sigma-Aldrich). LIPAM triglyceride lipase activity and phospholipase A₂ activity are demonstrated by analysis of the cleavage products isolated from the hydrolysis reaction mixture.

Vesicles containing 1-palmitoyl-2-[1-¹⁴C]oleoyl phosphatidylcholine (Amersham Pharmacia Biotech.) are prepared by mixing 2.0 μCi of the radiolabeled phospholipid with 12.5 mg of unlabeled 1-palmitoyl-2-oleoyl phosphatidylcholine and drying the mixture under N₂. 2.5 ml of 150 mM Tris-HCl, pH 7.5, is added, and the mixture is sonicated and centrifuged. The supernatant may be stored at 4° C. The final reaction mixtures contain 0.25 ml of Hanks buffered salt solution supplemented with 2.0 mM taurochenodeoxycholate, 1.0% bovine serum albumin, 1.0 mM CaCl₂, pH 7.4, 150 μg of 1-palmitoyl-1-2-[1-¹⁴C]oleoyl phosphatidylcholine vesicles, and various amounts of LIPAM diluted in PBS. After incubation for 30 min at 37° C., 20 μg each of lyso-phosphatidylcholine and oleic acid are added as carriers and each sample is extracted for total lipids. The lipids are separated by thin layer chromatography using a two solvent system of chloroform:methanol:acetic acid:water (65:35:8:4) until the solvent front is, halfway up the plate. The process is then continued with hexane:ether:acetic acid (86:16:1) until the solvent front is at the top of the plate. The lipid-containing areas are visualized with I₂ vapor; the spots are scraped, and their radioactivity is determined by scintillation counting. The amount of radioactivity released as fatty acids will increase as a greater amount of LIPAM is added to the assay mixture while the amount of radioactivity released as lysophosphatidylcholine will remain low. This demonstrates that LIPAM cleaves at the sn-2 and not the sn-1 position, as is characteristic of phospholipase A₂ activity.

In the alternative, phospholipase activity of LIPAM is measured by the hydrolysis of a fatty acyl residue at the sn-1 position of phosphatidylserine. LIPAM is combined with the tritium [³H] labeled substrate phosphatidylserine at stoichiometric quantities in a suitable buffer. Following an appropriate incubation time, the hydrolyzed reaction products are separated from the substrates by chromatographic methods. The amount of acylglycerophosphoserine produced is measured by counting tritiated product with the help of a scintillation counter. Various control groups are set up to account for background noise and unincorporated substrate. The final counts represent the tritiated enzyme product [³H]-acylglycerophosphoserine, which is directly proportional to the activity of LIPAM in biological samples.

Lipoxygenase activity of LIPAM can be measured by chromatographic methods. Extracted LIPAM lipoxygenase protein is incubated with 100 μM [1-¹⁴C] arachidonic acid or other unlabeled fatty acids at 37° C. for 30 min. After the incubation, stop solution (acetonitrile:methanol:water, 350:150:1) is added. The samples are extracted and analyzed by reverse-phase HPLC using a solvent system of methanol/water/acetic acid, 85:15:0.01 (vol/vol) at a flow rate of 1 ml/min. The effluent is monitored at 235 nm and analyzed for the presence of the major arachidonic metabolite such as 12-HPETE (catalyzed by 12-LOX). The fractions are also subjected to liquid scintillation counting. The final counts represent the products, which is directly proportional to the activity of LIPAM in biological samples. For stereochemical analysis, the metabolites of arachidonic acid are analyzed further by chiral phase-HPLC and by mass spectrometry (Sun, D. et al. (1998) J. Biol. Chem. 273:33540-33547).

Sialidase activity of LIPAM is assayed using various substrates, including but not limited to 2′-(4-methylumbelliferyl)α-D-N-acetylneuramic acid, 2′-O-(o-nitrophenyl)α-D-N-acetylneuramic acid, 2′-O-(p-nitrophenyl)α-D-N-acetylneuramic acid, and α(2-3)- and α(2-6)-sialyllactose. The reaction mixture contains 30 nmol substrate, 0.2 mg bovine serum albumin, 10 μmol sodium acetate (pH 4.6), 0.2 mg Triton X-100, and purified LIPAM (or a sample containing LIPAM). Following incubation at 37° C. for 10-30 min the released sialic acid is quantified using the thiobarbituric acid method (Aminoff, D. (1961) Biochem. J. 81:384-392 ). One unit of sialidase activity is defined as the amount of LIPAM that catalyzes the release of 1 nmol of sialic acid from substrate per hour (Hasegawa, T. et al. (2000) J. Biol. Chem. 275:8007-8015).

DHAPAT activity of LIPAM can be determined by measuring the rate of incorporation of radioactivity from radioactive dihydroxyacetone phosphate into chloroform-soluble products (Bates, E. J. and Saggerson, E. D. (1979) Biochem J. 182:751-762). Radioactive dihydroxyacetone phosphate is generated in the reaction mixture from [U-¹⁴C]fructose 1,6-bisphosphate by reacting with the coupling enzymes aldolase and triose phosphate isomerase. DHAPAT activity can be measured at 30° C. in a final volume of 1 ml containing: 120 mM KCl, 50 mM Tris/HCl buffer, pH 7.4, 4 mM MgCl₂, 8 mM NaF, fatty acid-poor albumin (4 mg/ml), 65 μM palmitoyl-CoA, 0.5 mM [U-¹⁴C]fructose 1,6-bisphosphate (0.4 μCi/ml), 50 μg aldolase (0.45 unit), and 3 μg triose phosphate isomerase (15 units), giving a concentration of 0.45 mM dihydroxyacetone phosphate in the reaction mixture. Before use, the coupling enzymes were dialyzed at 4° C. overnight against 750 vol. of 240 mM KCl/100 mM Tris/HCl buffer, pH 7.4, to remove (NH₄)₂SO₄. A 0.9 ml portion of reaction mixture is preincubated for 16 minutes at 30° C. A 0.1 ml portion of LIPAM is then added to the reaction mixture. After 6 to 8 minutes further incubation, the reaction is terminated by adding 3.5 ml chloroform/methanol (1:2, v/v). The mixture is centrifuged for 5 minutes at 1500 g, the supernatant is decanted, and 1.0 ml chloroform is added followed by 1.0 ml 2 M KCl in 0.2 M H₃PO₄. After mixing, the mixture is centrifuged for 5 minutes at 1000 g, and the top layer is discarded. The lower chloroform layer is washed with 4 ml water and 0.5 ml 2 M KCl in 0.2 M H₃PO₄ and centrifuged again for 5 minutes at 1000 g. A 1.0 ml portion of the chloroform layer is evaporated to dryness in a glass scintillation vial. Liquid-scintillation counting of the samples is performed in toluene containing 2,5-bis-(5-t-butylbenzoxazol-2-yl)thiophen (4 g/liter). The amount of radioactivity incorporated into chloroform-soluble products is proportional to the amount of LIPAM in the sample.

The transfer rate of lipid by LIPAM between lipoproteins is determined by monitoring the fluorescence spectrum of pyrene-lipid during the reaction. Human plasma lipoproteins are labeled with pyrene-lipids. Cholesterol 1-pyrenehexanoate (pyrene-CE) (Sigma and Molecular Probes) and triolein (Sigma) are mixed with phosphatidylcholine in the starting milligram weight ratio of 1:1:2. Control microemulsion is prepared from triolein, cholesteryl oleate, and phosphatidylcholine with the starting milligram weight ratio of 1:1:2. Donor LDL and HDL are labeled according to Main, L. A. et al. (1998; J. Biochem. 124:237-243). Acceptor lipoproteins are either untreated lipoproteins or prepared as in the donor lipoproteins except that they are incubated in the emulsion which does not contain pyrene-lipid. Donor and acceptor lipoproteins of the pyrene-lipid are mixed at 37° C. and LIPAM is added. Flurescence emmision is monitored at 396 and 468 nm upon, excitation at 320 nm. The ratio of the emission fluorescence intensities at the two wavelengths is an indicator of the pyrene-lipid content in the donor particles (Main et al. supra)

Various modifications and variations of the described compositions, methods, and systems of the invention will be apparent to those skilled in the art without departing from the scope and spirit of the invention. It will be appreciated that the invention provides novel and useful proteins, and their encoding polynucleotides, which can be used in the drug discovery process, as well as methods for using these-compositions for the detection, diagnosis, and treatment of diseases and conditions. Although the invention has been described in connection with certain embodiments, it should be understood that the invention as claimed should not be unduly limited to such specific embodiments. Nor should the description of such embodiments be considered exhaustive or limit the invention to the precise forms disclosed. Furthermore, elements from one embodiment can be readily recombined with elements from one or more other embodiments. Such combinations can form a number of embodiments within the scope of the invention. It is intended that the scope of the invention be defined by the following claims and their equivalents.

TABLE 1

| | | | | Incyte | |
|---|---|---|---|---|---|
| | Polypeptide | Incyte | Polynucleotide | Polynucleotide | |
| Incyte Project ID | SEQ ID NO: | Polypeptide ID | SEQ ID NO: | ID | Incyte Full Length Clones |
| 7511098 | 1 | 7511098CD1 | 22 | 7511098CB1 | 2344283CA2 |
| 7522037 | 2 | 7522037CD1 | 23 | 7522037CB1 | 95135610CA2 |
| 7524271 | 3 | 7524271CD1 | 24 | 7524271CB1 | 90030007CA2 |
| 7513132 | 4 | 7513132CD1 | 25 | 7513132CB1 | |
| 7513134 | 5 | 7513134CD1 | 26 | 7513134CB1 | |
| 7523653 | 6 | 7523653CD1 | 27 | 7523653CB1 | 95116625CA2, 95179069CA2, 95179742CA2 |
| 7751418 | 7 | 7751418CD1 | 28 | 7751418CB1 | |
| 7523952 | 8 | 7523952CD1 | 29 | 7523952CB1 | 95104475CA2 |
| 7513020 | 9 | 7513020CD1 | 30 | 7513020CB1 | 6479978CA2 |
| 7513162 | 10 | 7513162CD1 | 31 | 7513162CB1 | |
| 7513164 | 11 | 7513164CD1 | 32 | 7513164CB1 | |
| 7513496 | 12 | 7513496CD1 | 33 | 7513496CB1 | 90085680CA2 |
| 7514724 | 13 | 7514724CD1 | 34 | 7514724CB1 | 90207993CA2, 90208631CA2 |
| 7514797 | 14 | 7514797CD1 | 35 | 7514797CB1 | 95031472CA2 |
| 7512100 | 15 | 7512100CD1 | 36 | 7512100CB1 | 90007336CA2 |
| 7512101 | 16 | 7512101CD1 | 37 | 7512101CB1 | 90007344CA2 |
| 7516771 | 17 | 7516771CD1 | 38 | 7516771CB1 | 95057885CA2 |
| 7512128 | 18 | 7512128CD1 | 39 | 7512128CB1 | 90010465CA2 |
| 7518098 | 19 | 7518098CD1 | 40 | 7518098CB1 | |
| 7524729 | 20 | 7524729CD1 | 41 | 7524729CB1 | |
| 7520475 | 21 | 7520475CD1 | 42 | 7520475CB1 | |

TABLE 2

| Polypeptide | Incyte | GenBank ID NO: | | |
|---|---|---|---|---|
| SEQ | Polypeptide | or PROTEOME | Probability | |
| ID NO: | ID | ID NO: | Score | Annotation |
| 1 | 7511098CD1 | g20196199 | 1.4E−33 | [Homo sapiens] saposin-like protein |
| | | 569176\|TMEM4 | 1.0E−34 | [Homo sapiens][Plasma membrane; Unspecified membrane] Transmembrane |
| | | | | protein 4, a putative type II membrane protein |
| | | | | Yokoyama-Kobayashi, M. et al., Selection of cDNAs encoding putative type II |
| | | | | membrane proteins on the cell surface from a human full-length cDNA bank., |
| | | | | Gene 228, 161-7 (1999) |
| | | 609046\|Tmem4 | 5.7E−34 | [Mus musculus][Unspecified membrane] Protein with very strong similarity to |
| | | | | human TMEM4, which is a putative type II membrane protein |
| 2 | 7522037CD1 | g178836 | 1.5E−36 | [Homo sapiens] apolipoprotein C-II |
| | | | | Wei, C. F. et al., The structure of the human apolipoprotein C-II gene. Electron |
| | | | | microscopic analysis of RNA:DNA hybrids, complete nucleotide sequence, and |
| | | | | identification of 5′ homologous sequences among apolipoprotein genes, J. Biol. |
| | | | | Chem. 260, 15211-15221 (1985) |
| | | | | Fojo, S. S. et al., Donor splice site mutation in the apolipoprotein (Apo) C-II gene |
| | | | | (Apo C-IIHamburg) of a patient with Apo C-II deficiency, J. Clin. Invest. 82, |
| | | | | 1489-1494 (1988). |
| | | 343268\|APOC2 | 1.1E−28 | [Homo sapiens][Hydrolase] Apolipoprotein C-II, cofactor and activator for |
| | | | | lipoprotein lipase (LPL), which hydrolyzes triglyceride-rich lipoproteins; gene |
| | | | | mutations cause hypercholesterolemia, hypertriglyceridemia, |
| | | | | hyperlipoproteinemia, and chylomicronemia syndrome |
| | | | | Inadera, H. et al., A missense mutation (Trp 26-->Arg) in exon 3 of the |
| | | | | apolipoprotein CII gene in a patient with apolipoprotein CII deficiency (apo CII- |
| | | | | Wakayama)., Biochem Biophys Res Commun 193, 1174-83 (1993) |
| | | 584271\|Apoc2 | 6.5E−17 | [Mus musculus] Apolipoprotein C2, activator for lipoprotein lipase (Lp1), which |
| | | | | hydrolyzes triglyceride-rich lipoproteins; mutations in human APOC2 gene cause |
| | | | | hypercholesterolemia, hypertriglyceridemia, hyperlipoproteinemia, and |
| | | | | chylomicronemia syndrome |
| | | | | Hoffer, M. J. et al., Structure and expression of the mouse apolipoprotein C2 |
| | | | | gene., Genomics 17, 45-51 (1993). |
| 3 | 7524271CD1 | g338298 | 4.8E−97 | [Homo sapiens] sufactant apoprotein 18 precursor |
| | | | | Revak, S. D. et al., Use of human surfactant low molecular weight apoproteins in |
| | | | | the reconstitution of surfactant biologic activity, J. Clin. Invest. 81, 826-833 |
| | | | | (1988) |
| | | 344814\|SFTPB | 1.2E−97 | [Homo sapiens][Extracellular (excluding cell wall)] Pulmonary-associated protein |
| | | | | B surfactant, a component of the pulmonary surfactant complex required for |
| | | | | normal respiration; mutation of the corresponding gene causes familial alveolar |
| | | | | proteinosis and misalignment of lung vessels |
| | | | | Luzi, P. et al., DNA binding proteins that amplify surfactant protein B gene |
| | | | | expression: isolation and characterization., Biochem Biophys Res Commun 208, |
| | | | | 153-60 (1995). |
| 4 | 7513132CD1 | g190038 | 0.0 | [Homo sapiens] phospholipase C-gamma |
| | | | | Burgess, W. H. et al., Characterization and cDNA cloning of phospholipase C- |
| | | | | gamma, a major substrate for heparin-binding growth factor 1 (acidic fibroblast |
| | | | | growth factor)-activated tyrosine kinase, Mol. Cell. Biol. 10, 4770-4777 (1990). |
| | | 337016\|PLCG1 | 0.0 | [Homo sapiens][Hydrolase][Plasma membrane] Phospholipase C gamma 1, |
| | | | | activated by heparin-binding growth factor 1-activated tyrosine kinase, involved in |
| | | | | intracellular calcium signaling |
| | | | | Thodeti, C. K. et al. Leukotriene D(4) triggers an association between |
| | | | | gbetagamma subunits and phospholipase C-gamma1 in intestinal epithelial cells. J |
| | | | | Biol Chem 275, 9849-53 (2000). |
| | | 590481\|Plcg1 | 0.0 | [Rattus norvegicus][Hydrolase] Phospholipase C gamma 1, member of a family of |
| | | | | G-protein-regulated phospholipases that hydrolyze phosphatidylinositol 4,5- |
| | | | | bisphosphate |
| | | | | Suh, P. G. et al., Inositol phospholipid-specific phospholipase C: complete cDNA |
| | | | | and protein sequences and sequence homology to tyrosine kinase-related |
| | | | | oncogene products. Proc Natl Acad Sci USA 85, 5419-23 (1988). |
| 5 | 7513134CD1 | g190038 | 0.0 | [Homo sapiens] phospholipase C-gamma |
| | | | | Burgess, W. H. et al. (supra) |
| | | 337016\|PLCG1 | 0.0 | [Homo sapiens][Hydrolase][Plasma membrane] Phospholipase C gamma 1, |
| | | | | activated by heparin-binding growth factor 1-activated tyrosine kinase, involved in |
| | | | | intracellular calcium signaling |
| | | | | Chou, T. T. et al., A novel apoptotic pathway induced by nerve growth factor- |
| | | | | mediated TrkA activation in medulloblastoma., J Biol Chem 275, 565-70 (2000). |
| | | 590481\|Plcg1 | 0.0 | [Rattus norvegicus][Hydrolase] Phospholipase C gamma 1, member of a family of |
| | | | | G-protein-regulated phospholipases that hydrolyze phosphatidylinositol 4,5- |
| | | | | bisphosphate |
| | | | | Venema, R. C. et al., Angiotensin II-induced association of phospholipase |
| | | | | Cgamma1 with the G-protein-coupled AT1 receptor., J Biol Chem 273, 7703-8 |
| | | | | (1998). |
| 6 | 7523653CD1 | g180260 | 2.9E−223 | [Homo sapiens] cholesteryl ester transfer protein precursor |
| | | | | Drayna, D. et al., Cloning and sequencing of human cholesteryl ester transfer |
| | | | | protein cDNA, Nature 327, 632-634 (1987). |
| | | 339214\|CETP | 2.1E−224 | [Homo sapiens][Transferase; Structural protein] Cholesteryl ester transfer protein, |
| | | | | transfers cholesteryl esters from high density lipoproteins to other lipoproteins; |
| | | | | deficiency is associated with increased coronary heart disease despite increased |
| | | | | HDL levels |
| | | | | Oliveira, H. C. F. et al., Human cholesteryl ester transfer protein gene proximal |
| | | | | promoter contains dietary cholesterol positive responsive elements and mediates |
| | | | | expression in small intestine and periphery while predominant liver and spleen |
| | | | | expression is controlled by 5′-distal sequences. Cis-acting sequences mapped in |
| | | | | transgenic mice., J Biol Chem 271, 31831-8 (1996). |
| | | 581929\|Lbp | 7.8E−16 | [Mus musculus][Extracellular (excluding cell wall)] Lipopolysaccharide (LPS)- |
| | | | | binding protein, an acute phase protein with bactericidal activity against gram- |
| | | | | negative bacteria, protects against septic shock |
| | | | | Fierer, J. et al., The role of lipopolysaccharide binding protein in resistance to |
| | | | | Salmonella infections in mice., J Immunol 168, 6396-403. (2002). |
| 7 | 7751418CD1 | g10764778 | 4.6E−77 | [Homo sapiens] phosphoinositol 3-phosphate-binding protein-2 |
| | | | | Dowler, S. et al., Identification of pleckstrin-homology-domain-containing |
| | | | | proteins with novel phosphoinositide-binding specificities, Biochem. J. 351 (Pt 1), |
| | | | | 19-31 (2000). |
| | | 789815\|PEPP2 | 3.3E−78 | [Homo sapiens] Phosphoinositol 3-phosphate-binding protein-2, contains a |
| | | | | pleckstrin homology domain with a putative phosphatidylinositol 3,4,5- |
| | | | | trisphosphate-binding motif and two WW domains, a probable phospholipid |
| | | | | binding protein which may act as an adaptor protein |
| | | | | Dowler, S. et al. (supra) |
| 8 | 7523952CD1 | g468326 | 1.9E−29 | [Homo sapiens] phospholipid transfer protein |
| | | | | Day, J. R. et al., Complete cDNA encoding human phospholipid transfer protein |
| | | | | from human endothelial cells, J. Biol. Chem. 269, 9388-9391 (1994) |
| | | 343664\|PLTP | 1.4E−30 | [Homo sapiens][Transporter] Phospholipid transfer protein, functions in |
| | | | | phospholipid transport and conversion of high density lipoproteins into larger and |
| | | | | smaller particles, level of activity is altered in emphysema, obesity and diabetes, |
| | | | | may play a role in atherogenesis |
| | | | | Kawano, K. et al. (supra) |
| | | 585565\|Pltp | 3.3E−24 | [Mus musculus][Transporter][Extracellular (excluding cell wall)] Phospholipid |
| | | | | transfer protein, functions in phospholipid transport and conversion of high |
| | | | | density lipoproteins into larger and smaller particles; human PLTP activity is |
| | | | | altered in emphysema, obesity and diabetes and it may play a role in atherogenesis |
| | | | | Jiang, X. C. et al., Regulation of murine plasma phospholipid transfer protein |
| | | | | activity and mRNA levels by lipopolysaccharide and high cholesterol diet., J Biol |
| | | | | Chem 270, 17133-8 (1995). |
| 9 | 7513020CD1 | g2584769 | 0.0 | [Homo sapiens] dihydroxyacetone phosphate acyltransferase (DHAPAT) |
| | | | | Thai, T. P. et al. Ether lipid biosynthesis: isolation and molecular characterization |
| | | | | of human dihydroxyacetonephosphate acyltransferase. FEBS Lett. 420, 205-211 |
| | | | | (1997). |
| | | | | Thai, T. P. et al. Synthesis of plasmalogens in eye lens epithelial cells. FEBS Lett. |
| | | | | 456, 263-268 (1999). |
| | 7513020CD1 | 569138\|GNPAT | 0.0 | [Homo sapiens][Transferase][Cytoplasmic; Peroxisome] Glyceronephosphate O |
| | | | | acyltransferase (Acyl-CoA: dihydroxyacetonephosphate acyltransferase), a key |
| | | | | enzyme of plasmalogen biosynthesis; mutations in the GNPAT gene are |
| | | | | associated with Rhizomelic chondrodysplasia punctata (RCDP) type 2 |
| | | | | Ofman, R. et al. Acyl-CoA: dihydroxyacetonephosphate acyltransferase: cloning of |
| | | | | the human cDNA and resolution of the molecular basis in rhizomelic |
| | | | | chondrodysplasia punctata type 2. Hum Mol Genet 7, 847-53 (1998). |
| | | | | Hajra, A. K. Dihydroxyacetone phosphate acyltransferase. Biochim. Biophys. |
| | | | | Acta 1348, 27-34 (1997). |
| | 7513020CD1 | 429862\|Gnpat | 1.7E−271 | [Mus musculus][Transferase][Cytoplasmic; Peroxisome] Glyceronephosphate O |
| | | | | acyltransferase (Acyl-CoA:dihydroxyacetonephosphate acyltransferase), a key |
| | | | | enzyme of plasmalogen biosynthesis; mutations in the human GNPAT gene are |
| | | | | associated with Rhizomelic chondrodysplasia punctata (RCDP) type 2 |
| | | | | Ofman, R. et al. Identification and characterization of the mouse cDNA encoding |
| | | | | acyl-CoA:dihydroxyacetone phosphate acyltransferase. Biochim Biophys Acta |
| | | | | 1439, 89-94 (1999). |
| 10 | 7513162CD1 | g1690 | 0.0 | [Oryctolagus cuniculus] Phospholipase |
| | | | | Boll, W. et al. Messenger RNAs expressed in intestine of adult but not baby |
| | | | | rabbits. Isolation of cognate cDNAs and characterization of a novel brush border |
| | | | | protein with esterase and phospholipase activity. J. Biol. Chem. 268, 12901-12911 |
| | | | | (1993). |
| | | 331260\|Rn.10866 | 0.0 | [Rattus norvegicus][Hydrolase] Intestinal phospholipase B/lipase, displays broad |
| | | | | lipolytic activities, has phospholipase A2, lysophospholipase, and triacylglycerol |
| | | | | lipase properties; compensates for the depletion of pancreatic lipolytic enzymes in |
| | | | | rats with pancreas insufficiency |
| | | | | Tchoua, U. et al. Increased intestinal phospholipase A(2) activity catalyzed by |
| | | | | phospholipase B/lipase in WBN/Kob rats with pancreatic insufficiency. Biochim |
| | | | | Biophys Acta 1487, 255-67. (2000). |
| | | 443847\| | 1.3E−63 | [Caenorhabditis elegans] Putative paralog of C. elegans W02B12.1 |
| | | Y65B4BR.1 | | |
| | | | | Bateman, A. et al. Pfam 3.1: 1313 multiple alignments and profile HMMs match |
| | | | | the majority of proteins. Nucleic Acids Res 27, 260-2 (1999). |
| 11 | 7513164CD1 | g1690 | 0.0 | [Oryctolagus cuniculus] Phospholipase |
| | | | | Boll, W. et al. (supra) |
| | | 331260\|Rn.10866 | 0.0 | [Rattus norvegicus][Hydrolase] Intestinal phospholipase B/lipase, displays broad |
| | | | | lipolytic activities, has phospholipase A2, lysophospholipase, and triacylglycerol |
| | | | | lipase properties; compensates for the depletion of pancreatic lipolytic enzymes in |
| | | | | rats with pancreas insufficiency |
| | | | | Takemori, H. et al. Identification of functional domains of rat intestinal |
| | | | | phospholipase B/lipase. Its cDNA cloning, expression, and tissue distribution. J |
| | | | | Biol Chem 273, 2222-31 (1998). |
| | | 443847\| | 8.0E−65 | [Caenorhabditis elegans] Putative paralog of C. elegans W02B12.1 |
| | | Y65B4BR.1 | | |
| | | | | Bateman, A. et al. (supra) |
| 12 | 7513496CD1 | g12408013 | 5.0E−196 | [Homo sapiens] apolipoprotein L-I |
| | | | | Duchateau, P. N. et al. Apolipoprotein L, a new human high density lipoprotein |
| | | | | apolipoprotein expressed by the pancreas. Identification, cloning, characterization, |
| | | | | and plasma distribution of apolipoprotein L. J. Biol. Chem. 272, 25576-25582 |
| | | | | (1997) |
| | | 613517\|APOL1 | 3.6E−190 | [Homo sapiens][Transporter][Extracellular (excluding cell wall)] Apolipoprotein |
| | | | | L, a component of large, apoA-I(APOA1)-containing, high density lipoproteins, |
| | | | | may be involved in lipid transport and metabolism |
| | | | | Duchateau, P. N. et al. Apolipoprotein L gene family. Tissue-specific expression, |
| | | | | splicing, promoter regions; discovery of a new gene. J. Lipid Res. 42, 620-630 |
| | | | | (2001). |
| | | 703635\|APOL2 | 1.1E−99 | [Homo sapiens] Apolipoprotein L 2, a putative member of the apolipoprotein L |
| | | | | family of proteins with possible roles in lipid exchange and transport |
| | | | | Page, N. M. et al. The human apolipoprotein 1 gene cluster: identification, |
| | | | | classification, and sites of distribution. Genomics 74, 71-78 (2001). |
| 13 | 7514724CD1 | g206459 | 6.6E−14 | [Rattus norvegicus] prepulmonary surfactant-associated protein A |
| | | | | Sano, K. et al. Isolation and sequence of a cDNA clone for the rat pulmonary |
| | | | | surfactant-associated protein (PSP-A). Biochem. Biophys. Res. Commun. 144, |
| | | | | 367-374 (1987) |
| | | 772430\|Sftpa | 2.3E−14 | [Mus musculus] Surfactant-associated protein A1, component of the surfactant |
| | | | | complex that functions in tubular myelin formation within lung alveoli, and has a |
| | | | | role in pathogen defense; reduced expression of human SFTPA1 is associated |
| | | | | with respiratory distress syndrome |
| | | | | Motwani, M. et al. Mouse surfactant protein-D. cDNA cloning, characterization, |
| | | | | and gene localization to chromosome 14. J. Immunol. 155, 5671-5677 (1995) |
| | | 591453\|Sftpa1 | 2.3E−14 | [Rattus norvegicus] Surfactant-associated protein A1, component of the surfactant |
| | | | | complex that has a role in pathogen defense and regulates phospholipid transport; |
| | | | | reduced expression of human SFTPA1 is associated with respiratory distress |
| | | | | syndrome |
| | | | | Smith, C. I. et al. Sequence of rat surfactant protein A gene and functional |
| | | | | mapping of its upstream region. Am. J. Physiol. 269, L603-612 (1995). |
| | | 690814\|SFTPA2 | 9.9E−14 | [Homo sapiens] Surfactant protein A2, member of a family of collagenous C type |
| | | | | lectins that is a component of pulmonary surfactant, essential for normal |
| | | | | respiratory function; polymorphisms may contribute to the etiology of respiratory |
| | | | | distress syndrome |
| | | | | Scavo, L. M. et al. Human surfactant proteins A1 and A2 are differentially |
| | | | | regulated during development and by soluble factors. Am. J. Physiol. 275, L653-669 |
| | | | | (1998). |
| 14 | 7514797CD1 | g564065 | 4.8E−152 | [Homo sapiens] peroxisomal enoyl-CoA hydratase-like protein |
| | | | | FitzPatrick, D. R. et al. Isolation and characterization of rat and human cDNAs |
| | | | | encoding a novel putative peroxisomal enoyl-CoA hydratase. Genomics 27, 457-466 |
| | | | | (1995). |
| | | 335116\|ECH1 | 3.8E−153 | [Homo sapiens][Lyase][Cytoplasmic; Peroxisome] Putative peroxisomal enoyl |
| | | | | Coenzyme A hydratase, may play a role in peroxisomal beta-oxidation |
| | | | | FitzPatrick, D. R. et al. (1995) supra |
| | | 587697\|Ech1 | 1.2E−119 | [Mus musculus][Lyase][Cytoplasmic; Peroxisome] Protein with strong similarity |
| | | | | to rat Ech1, which is a putative peroxisomal enoyl Coenzyme A hydratase that |
| | | | | may function in peroxisomal beta-oxidation |
| | | | | FitzPatrick, D. R. et al. (1995) supra |
| 15 | 7512100CD1 | g10953956 | 2.3E−90 | [Homo sapiens] sorting nexin 16 |
| | | | | Worby, C. A. and Dixon, J. E. Sorting out the cellular functions of sorting nexins. |
| | | | | Nat. Rev. Mol. Cell. Biol. 3: 919-931 (2002). |
| | | 626175\|SNX16 | 1.8E−91 | [Homo sapiens] Protein containing a phox protein (PX) domain, has a region of |
| | | | | moderate similarity to a region of cytokine-independent survival kinase (mouse |
| | | | | Cisk), which is a serine-threonine kinase that promotes IL-3-dependent survival of |
| | | | | hematopoietic cells |
| | | 627050\|Snx16 | 4.3E−86 | [Rattus norvegicus] Protein containing a phox protein (PX) domain, which bind |
| | | | | phosphoinositides, has strong similarity to uncharacterized human SNX16 |
| 16 | 7512101CD1 | g10953956 | 4.9E−107 | [Homo sapiens] sorting nexin 16 |
| | | | | Worby, C. A. and Dixon, J. E. (2002), supra. |
| | | 626175\|SNX16 | 3.8E−108 | [Homo sapiens] Protein containing a phox protein (PX) domain, has a region of |
| | | | | moderate similarity to a region of cytokine-independent survival kinase (mouse |
| | | | | Cisk), which is a serine-threonine kinase that promotes Il3-dependent survival of |
| | | | | hematopoietic cells |
| | | 627050\|Snx16 | 7.2E−98 | [Rattus norvegicus] Protein containing a phox protein (PX) domain, which bind |
| | | | | phosphoinositides, has strong similarity to uncharacterized human SNX16 |
| 17 | 7516771CD1 | g187152 | 4.5E−221 | [Homo sapiens] lysosomal acid lipase/cholesteryl esterase |
| | | | | Anderson, R. A. et al. Cloning and expression of cDNA encoding human |
| | | | | lysosomal acid lipase/cholesteryl ester hydrolase. Similarities to gastric and |
| | | | | lingual lipases. J. Biol. Chem. 266: 22479-22484 (1991). |
| | | 339478\|LIPA | 3.5E−222 | [Homo sapiens] [Hydrolase] [Lysosome/vacuole; Cytoplasmic] Lysosomal acid |
| | | | | lipase A (cholesteryl ester hydrolase), deacylates cholesteryl and triacylglyceryl |
| | | | | ester core lipids of low density lipoproteins in lysosomes; mutations in the gene |
| | | | | are associated with Wolman disease and cholesteryl ester storage disease |
| | | | | Anderson, R. A. et al. (1991), supra. |
| | | | | Anderson, R. A. et al. Mutations at the lysosomal acid cholesteryl ester hydrolase |
| | | | | gene locus in Wolman disease. Proc. Natl. Acad. Sci. USA 91: 2718-2722 (1994). |
| | | | | Anderson, R. A. et al. Lysosomal acid lipase mutations that determine phenotype |
| | | | | in Wolman and cholesterol ester storage disease. Mol. Genet. Metab. 68: 333-345 |
| | | | | (1999). |
| | | 777430\|Lipa | 4.6E−172 | [Rattus norvegicus] [Hydrolase] Carboxyl ester lipase, (cholesterol esterase), |
| | | | | enzyme that is stimulated by bile salt and plays a role in lipid metabolism, |
| | | | | phosphorylation is essential for secretion from the pancreas |
| | | | | Kissel, J. A. et al. Molecular cloning and expression of cDNA for rat pancreatic |
| | | | | cholesterol esterase. Biochim. Biophys. Acta. 1006: 227-236 (1989). |
| | | | | Ghosh, S. et al. Molecular cloning and expression of rat hepatic neutral |
| | | | | cholesteryl ester hydrolase. Biochim. Biophys. Acta 1259: 305-312 (1995). |
| | | | | Pasqualini, E. et al. Phosphorylation of the rat pancreatic bile-salt-dependent |
| | | | | lipase by casein kinase II is essential for secretion. Biochem. J. 345: 121-128 |
| | | | | (2000). |
| 18 | 7512128CD1 | g3661595 | 1.7E−31 | [Arabidopsis thaliana] aminoalcoholphosphotransferase |
| | | | | Dewey, R. E. et al., Characterization of aminoalcoholphosphotransferases from |
| | | | | Arabidopsis thaliana and soybean, Plant Physiol. Biochem. 37, 445-457 (2000) |
| | | 730175\|KIAA1724 | 5.3E−134 | [Homo sapiens] Protein with low similarity to sn-1,2-diacylglycerol |
| | | | | ethanolaminephosphotransferase (S. cerevisiae Ept1p), which catalyzes the |
| | | | | synthesis of phosphatidylethanolamine from CDP-ethanolamine and |
| | | | | diacylglycerol |
| | | 243523\|F22E10.5 | 9.8E−30 | [Caenorhabditis elegans] Protein with high similarity to choline- |
| | | | | ethanolaminephosphotransferase (human CEPT1), which catalyzes a step in the |
| | | | | formation of phosphatidylcholine or phosphatidylethanolamine, member of the |
| | | | | CDP-alcohol phosphatidyltransferase family |
| 19 | 7518098CD1 | g4808601 | 6.9E−78 | [Homo sapiens] stearoyl-CoA desaturase |
| | | | | Zhang, L. et al., Human stearoyl-CoA desaturase: alternative transcripts generated |
| | | | | from a single gene by usage of tandem polyadenylation sites, Biochem. J. 340 (Pt |
| | | | | 1), 255-264 (1999) |
| | | 331434\|Rn.10982 | 1.3E−61 | [Rattus norvegicus][Oxidoreductase] Stearoyl-coenzyme A desaturase, a putative |
| | | | | enzyme that catalyzes the conversion of saturated fatty acids to the corresponding |
| | | | | monounsaturated fatty acids |
| | | 694494\|Scd3 | 1.6E−59 | [Mus musculus] Stearoyl-coenzyme A desaturase 3, a putative enzyme involved in |
| | | | | the conversion of saturated fatty acids into monounsaturated fatty acids, expressed |
| | | | | in sebaceous glands of the skin, most highly in males |
| 20 | 7524729CD1 | g4836419 | 9.6E−236 | [Homo sapiens] endothelial lipase |
| | | | | Hirata, K. et al., Cloning of a unique lipase from endothelial cells extends the |
| | | | | lipase gene family, J. Biol. Chem. 274, 14170-14175 (1999) |
| | | 343038\|LIPG | 6.9E−237 | [Homo sapiens][Hydrolase] Endothelial-derived lipase (lipase G), member of the |
| | | | | triacylglycerol lipase family, catalyzes the hydrolysis of phosphatidylcholine, may |
| | | | | play a role in lipoprotein metabolism, inflammation, and development of vascular |
| | | | | diseases like atherosclerosis |
| | | 429998\|Lipg | 3.1E−186 | [Mus musculus][Hydrolase] Endothelial-derived lipase (lipase G), member of the |
| | | | | triacylglycerol lipase family, putative phospholipase; human LIPG may play role |
| | | | | in development of atherosclerosis |
| 21 | 7520475CD1 | g762826 | 0.0 | [Homo sapiens] phospholipase C beta 4 |
| | | | | Alvarez, R. A. et al., cDNA sequence and gene locus of the human retinal |
| | | | | phosphoinositide-specific phospholipase-C beta 4 (PLCB4), Genomics 29, 53-61 |
| | | | | (1995). |
| | | 688974\|Plcb4 | 0.0 | [Rattus norvegicus] Phospholipase C beta 4, member of a G protein-regulated |
| | | | | family of phospholipases that hydrolyze phosphatidylinositol 4,5-bisphosphate to |
| | | | | the second messengers inositol 1,4,5-trisphosphate and diacylglycerol |
| | | | | Kim, M. J. et al., A cytosolic, galphaq- and betagamma-insensitive splice variant |
| | | | | of phospholipase C-beta4, J Biol Chem 273, 3618-24 (1998). |
| | | | | Lee, C. W. et al., Regulation of phospholipase C-beta 4 by ribonucleotides and the |
| | | | | alpha subunit of Gq, J Biol Chem 269,25335-8 (1994). |
| | | 337014\| | 0.0 | [Homo sapiens][Hydrolase] Phospholipase C beta 4, member of a G protein- |
| | | PLCB4 | | regulated family of phospholipases that hydrolyze phosphatidylinositol 4,5- |
| | | | | bisphosphate to the second messengers inositol 1,4,5-trisphosphate and |
| | | | | diacylglycerol |
| | | | | Drissi, H. et al., Activation of phospholipase C-beta1 via Galphaq/11 during |
| | | | | calcium mobilization by calcitonin gene-related peptide, J Biol Chem 273, 20168-74 |
| | | | | (1998). |

TABLE 3

| SEQ | Incyte | Amino | | |
|---|---|---|---|---|
| ID | Polypeptide | Acid | | Analytical Methods |
| NO: | ID | Residues | Signature Sequences, Domains and Motifs | and Databases |
| 1 | 7511098CD1 | 114 | signal_cleavage: M1-A20 | SPSCAN |
| | | | Signal Peptide: M1-G16, M1-W19, M1-A20, M1-S23, M1-D25 | HMMER |
| | | | Potential Phosphorylation Sites: S55 S65 S71 S97 S110 | MOTIFS |
| 2 | 7522037CD1 | 87 | signal_cleavage: M1-G22 | SPSCAN |
| | | | Signal Peptide: M1-G17, M1-E19, M1-G22, M1-Q25, M1-V20 | HMMER |
| | | | APOLIPOPROTEIN CII APOCII CHYLOMICRON VLDL PLASMA LIPID | BLAST_PRODOM |
| | | | TRANSPORT DEGRADATION PRECURSOR PD010424: P26-E87 | |
| | | | APOLIPOPROTEIN A-I DM02599 | BLAST_DOMO |
| | | | \|P02655\|1-100: M1-E87 | |
| | | | \|P12278\|1-100: M1-E86 | |
| | | | \|Q05020\|1-96: M1-E86 | |
| | | | \|P27916\|1-99: L5-E87 | |
| | | | Potential Phosphorylation Sites: S46 S50 | MOTIFS |
| 3 | 7524271CD1 | 248 | signal_cleavage: M1-A23 | SPSCAN |
| | | | Signal Peptide: M1-G19, M1-P20, M1-A23, M1-A24, M1-A31 | HMMER |
| | | | Saposin/surfactant protein-B A-type DOMAIN: S28-G61 | HMMER_SMART |
| | | | Saposins-like type B: S164-C233, P77-C148 | HMMER_SMART |
| | | | Saposin A-type domain: S28-G61 | HMMER_PFAM |
| | | | Surfactant protein B: F72-S149 | HMMER_PFAM |
| | | | GLYCOPROTEIN PROTEIN PRECURSOR SA. PD01469: C117-C148 | BLIMPS_PRODOM |
| | | | PROTEIN B SPB PULMONARY SURFACTANTASSOCIATED PRECURSOR | BLAST_PRODOM |
| | | | PROTEOLIPID SPLPHE SURFACE FILM PD010610: M150-L248 | |
| | | | PROTEIN B SPB PULMONARY SURFACTANTASSOCIATED PROTEOLIPID | BLAST_PRODOM |
| | | | SPLPHE SURFACE FILM GASEOUS PD008002: F72-S149 | |
| | | | PRECURSOR PROTEIN B GLYCOPROTEIN PROSAPOSIN SPB SULFATED SGP1 | BLAST_PRODOM |
| | | | SULFATATION SIGNAL PD004487: C32-W60 | |
| | | | PULMONARY SURFACTANT PROTEIN B DM03863 | BLAST_DOMO |
| | | | \|P07988\|261-380: L132-L248 | |
| | | | \|P50405\|252-376: L132-L248 | |
| | | | \|P15285\|245-369: L132-P246 | |
| | | | SAPOSIN REPEAT DM02041\|P07988\|91-259: D66-T131 | BLAST_DOMO |
| | | | Potential Phosphorylation Sites: S149 S195 | MOTIFS |
| | | | Potential Glycosylation Sites: N178 | MOTIFS |
| 4 | 7513132CD1 | 906 | PH (pleckstrin homology) domain: T33-E142, S489-F591 | HMMER_PFAM |
| | | | Phosphatidylinositol-specific phospholipase C, X domain: T321-K465 | HMMER_PFAM |
| | | | SH2 domain: W550-Y639, W668-Y741 | HMMER_PFAM |
| | | | SH3 domain: C794-M849 | HMMER_PFAM |
| | | | Pleckstrin homology domain: T33-T144, S489-H680 | HMMER_SMART |
| | | | Phospholipase C, catalytic domain (part);: D320-K464 | HMMER_SMART |
| | | | Src homology 2 domains: E548-R645, K666-Y747 | HMMER_SMART |
| | | | Src homology 3 domains: C794-V850 | HMMER_SMART |
| | | | Phospholipase C signature PR00390: P325-Q343, E351-G371, G448-K465 | BLIMPS_PRINTS |
| | | | SH2 domain signature PR00401: L555-L569, D580-T590, V592-G603, K604-Q614, | BLIMPS_PRINTS |
| | | | E730-H744 | |
| | | | PI3 kinase P85 regulatory subunit signature PR00678: R675-N697, N700-V717 | BLIMPS_PRINTS |
| | | | PHOSPHOLIPASE PHOSPHODIESTERASE HYDROLASE 1- | BLAST_PRODOM |
| | | | PHOSPHATIDYLINOSITOL-45-BISPHOSPHATE PHOSPHOINOSITIDE-SPECIFIC | |
| | | | DEGRADATION LIPID TRANSDUCER CALCIUM-BINDING | |
| | | | PD001214: M322-K465 | |
| | | | DOMAIN CALCIUM-BINDING PHOSPHOLIPASE DEGRADATION 1- | BLAST_PRODOM |
| | | | PHOSPHATIDYLINOSITOL-45-BISPHOSPHATE HYDROLASE LIPID TRANSDUCER | |
| | | | PHOSPHODIESTERASE GAMMA PD004439: L29-N311 | |
| | | | 1-PHOSPHATIDYLINOSITOL-45-BISPHOSPHATE PHOSPHODIESTERASE GAMMA | BLAST_PRODOM |
| | | | PLCGAMMA-1 PHOSPHOLIPASE CGAMMA-1 PLCII PLC148 HYDROLASE | |
| | | | PD018886: I756-A795 | |
| | | | DOMAIN DEGRADATION LIPID PHOSPHOLIPASE TRANSDUCER GAMMA | BLAST_PRODOM |
| | | | HYDROLASE 1-PHOSPHATIDYLINOSITOL-45-BISPHOSPHATE PLC-GAMMA-1 | |
| | | | CALCIUM-BINDING PD023748: L466-K549 | |
| | | | SRC HOMOLOGY 2 (SH2) DOMAIN | BLAST_DOMO |
| | | | DM00048\|P08487\|544-660: L544-N661 | |
| | | | 1-PHOSPHATIDYLINOSITOL-4,5-BISPHOSPHATE PHOSPHODIESTERASE D | BLAST_DOMO |
| | | | DM00855\|P08487\|71-500: G71-V501 | |
| | | | DM00855\|P16885\|63-486: G71-V501 | |
| | | | DM00855\|A53970\|67-522: E70-E468 Y481-V501 | |
| | | | Potential Phosphorylation Sites: S18, S40, S43, S126, S173, S250, S312, S348, S412, | MOTIFS |
| | | | S470, S482, S489, S514, S540, S612, S631, S705, S729, S733, S739, S902, T86, T125, | |
| | | | T199, T237, T385, T396, T523, T618, T791, T898, Y93, Y210, Y292, Y472, Y702 | |
| | | | EF-hand calcium-binding domain: D165-L177 | MOTIFS |
| 5 | 7513134CD1 | 1266 | Protein Kinase C, C2 domain: I1090-T1177 | HMMER_PFAM |
| | | | PH (pleckstrin homology) domain: T33-E142, S489-F591, A804-Q931 | HMMER_PFAM |
| | | | Phosphatidylinositol-specific phospholipase C, X domain: T321-K465 | HMMER_PFAM |
| | | | Phosphatidylinositol-specific phospholipase C, Y domain: E952-R1070 | HMMER_PFAM |
| | | | Src homology 2 (SH2) domain: W550-Y639, W668-Y741 | HMMER_PFAM |
| | | | Src homology 3 (SH3) domain: C794-M849 | HMMER_PFAM |
| | | | Protein kinase C conserved region 2 (CalB): A1089-L1192 | HMMER_SMART |
| | | | Pleckstrin homology domain: T33-T144, S489-H680, A804-A933 | HMMER_SMART |
| | | | Phospholipase C, catalytic domain (part); domain X: D320-K464 | HMMER_SMART |
| | | | Phospholipase C, catalytic domain (part); domain Y: L953-R1070 | HMMER_SMART |
| | | | Src homology 2 domains: E548-R645, K666-Y747 | HMMER_SMART |
| | | | Src homology 3 domains: C794-V850 | HMMER_SMART |
| | | | Phospholipase C signature PR00390: P325-Q343, E351-G371, G448-K465, L1008-W1029, | BLIMPS_PRINTS |
| | | | W1029-M1047, F1178-R1188 | |
| | | | SH2 domain signature PR00401: D580-T590, V592-G603, K604-Q614, E730-H744 | BLIMPS_PRINTS |
| | | | C2 domain proteins PF00168: L1173-E1198 | BLIMPS_PFAM |
| | | | PHOSPHOLIPASE PHOSPHODIESTERASE HYDROLASE 1- | BLAST_PRODOM |
| | | | PHOSPHATIDYLINOSITOL-45-BISPHOSPHATE PHOSPHOINOSITIDE-SPECIFIC | |
| | | | DEGRADATION LIPID TRANSDUCER CALCIUM-BINDING PD001214: M322-K465 | |
| | | | DOMAIN CALCIUM-BINDING PHOSPHOLIPASE DEGRADATION 1- | BLAST_PRODOM |
| | | | PHOSPHATIDYLINOSITOL-45-BISPHOSPHATE HYDROLASE LIPID TRANSDUCER | |
| | | | PHOSPHODIESTERASE GAMMA PD004439: L29-N311 | |
| | | | DOMAIN PHOSPHOLIPASE TRANSDUCER GAMMA 1- | BLAST_PRODOM |
| | | | PHOSPHATIDYLINOSITOL-45-BISPHOSPHATE LIPID HYDROLASE | |
| | | | PHOSPHODIESTERASE CALCIUM-BINDING SH3 PD013158: E848-L951 | |
| | | | DOMAIN DEGRADATION LIPID PHOSPHOLIPASE TRANSDUCER GAMMA | BLAST_PRODOM |
| | | | HYDROLASE 1-PHOSPHATIDYLINOSITOL-45-BISPHOSPHATE PLC-GAMMA-1 | |
| | | | CALCIUM-BINDING PD023748: L466-K549 | |
| | | | 1-PHOSPHATIDYLINOSITOL-4,5-BISPHOSPHATE PHOSPHODIESTERASE D | BLAST_DOMO |
| | | | DM00712\|P08487\|921-1211: D921-F1212 | |
| | | | 1-PHOSPHATIDYLINOSITOL-4,5-BISPHOSPHATE PHOSPHODIESTERASE D | BLAST_DOMO |
| | | | DM00855\|P08487\|71-500: G71-V501 | |
| | | | DM00855\|P16885\|63-486: G71-V501 | |
| | | | DM00855\|A53970\|67-522: E70-E468 Y481-V501 | |
| | | | Potential Phosphorylation Sites: S18, S40, S43, S126, S173, S250, S312, S348, S412, | MOTIFS |
| | | | S470, S482, S489, S514, S540, S612, S631, S705, S729, S733, S739, S915, S982, | |
| | | | S1021, S1081, S1123, S1150, S1221, T86, T125, T199, T237, T385, T396, | |
| | | | T523, T618, T791, T972, | |
| | | | T986, T1056, T1068, Y93, Y210, Y292, Y472, Y702, Y977 | |
| | | | Potential Glycosylation Sites: N1195 | MOTIFS |
| | | | EF-hand calcium-binding domain: D165-L177 | MOTIFS |
| 6 | 7523653CD1 | 433 | Signal Peptide: M1-A17 | HMMER |
| | | | signal_cleavage: M1-A17 | SPSCAN |
| | | | LBP/BPI/CETP family, N-terminal domain: H24-T241 | HMMER_PFAM |
| | | | LBP/BPI/CETP family, C-terminal domain: F190-F420 | HMMER_PFAM |
| | | | BPI/LBP/CETP N-terminal domain: R31-F258 | HMMER_SMART |
| | | | BPI/LBP/CETP C-terminal domain: S224-M416 | HMMER_SMART |
| | | | Lipid-binding serum glycoprotein IPB001124: A3-C30, Q53-I99, M211-Q254 | BLIMPS_BLOCKS |
| | | | LBP/BPI/CETP family signature: A4-N78 | PROFILESCAN |
| | | | LIPID SIGNAL GLYCOPROTEIN PRECURSOR TRANSPORT TRANSFER ANTIBIOTIC | BLAST_PRODOM |
| | | | TRANSMBEMBRANE LIPOPOLYSACCHARIDE-BINDING PD006440: | |
| | | | V6-E246 N237-F420 | |
| | | | LIPOPOLYSACCHARIDE-BINDING PROTEIN | BLAST_DOMO |
| | | | DM02253\|P17213\|11-486: G27-K304 E267-D417 | |
| | | | DM02253\|P17453\|7-481: A4-E246 I264-I400 | |
| | | | DM02253\|P18428\|5-474: L7-E246 | |
| | | | Potential Phosphorylation Sites: S56, S89, S136, S150, S224, S243, S333, S340, S357, | MOTIFS |
| | | | S380, S396, T22, T44, T113, T191, T260, T319, T347 | |
| | | | Potential Glycosylation Sites: N105, N298, N353 | MOTIFS |
| | | | LBP/BPI/CETP family signature: A26-P58 | MOTIFS |
| 7 | 7751418CD1 | 1076 | PH (pleckstrin homology) domain: V119-Q236 | HMMER_PFAM |
| | | | Pleckstrin homology domain: V119-L238 | HMMER_SMART |
| | | | Potential Phosphorylation Sites: S62, S84, S98, S154, S180, S184, S204, S242, S263, | MOTIFS |
| | | | S301, S320, S380, S419, S430, S462, S510, S523, S562, S566, S579, S605, S634, | |
| | | | S643, S678, S789, S810, S885, S907, S915, S932, S941, S1001, S1011, | |
| | | | S1027, S1060, T220, T374, | |
| | | | T543, T549, T625, T723, T745, T829, T856, T978, T992, T1049, Y299, Y610, Y1017 | |
| | | | Potential Glycosylation Sites: N198, N259, N361, N577 | MOTIFS |
| | | | Leucine zipper pattern: L662-L683 | MOTIFS |
| | | | ATP/GTP-binding site motif A (P-loop): A598-S605 | MOTIFS |
| 8 | 7523952CD1 | 98 | Signal Peptide: M1-A17, M1-G21, M1-124, M1-C22, M1-K23 | HMMER |
| | | | signal_cleavage: M1-A17 | SPSCAN |
| | | | Lipid-binding serum glycoprotein IPB001124: P20-E47 | BLIMPS_BLOCKS |
| | | | LBP/BPI/CETP family signature: F4-R72 | PROFILESCAN |
| | | | LIPOPOLYSACCHARIDE-BINDING PROTEIN | BLAST_DOMO |
| | | | DM02253\|P55058\|1-464: M1-E67 | |
| | | | DM02253\|I49370\|1-464: M1-E67 | |
| | | | Potential Phosphorylation Sites: T27, T50, T92, Y62 | MOTIFS |
| | | | Potential Glycosylation Sites: N64 | MOTIFS |
| | | | LBP/BPI/CETP family signature: P20-P52 | MOTIFS |
| 9 | 7513020CD1 | 619 | Acyltransferase: L86-S282 | HMMER_PFAM |
| | | | AGP_acyltrn: 1-acyl-sn-glycerol-3-phosphate acyltransferases: G82-S222 | HMMER_TIGRFAM |
| | | | DIHYDROXYACETONE PHOSPHATE ACYLTRANSFERASE EC 2.3.1.42 DAPAT | BLAST_PRODOM |
| | | | GLYCERONEPHOSPHATE OACYLTRANSFERASE TRANSFERASE PEROXISOME | |
| | | | DISEASE MUTATION PD138790: S275-L619 | |
| | | | ACYLTRANSFERASE TRANSFERASE GLYCEROL3PHOSPHATE GPAT | BLAST_PRODOM |
| | | | PHOSPHOLIPID BIOSYNTHESIS MITOCHONDRIAL PRECURSOR | |
| | | | TRANSMEMBRANE MITOCHONDRION PD025192: S28-A570 | |
| | | | GLYCEROL; ACYLTRANSFERASE; DM08300 | BLAST_DOMO |
| | | | \|P44857\|185-805: S34-N470 | |
| | | | \|P00482\|205-826: I45-A435 | |
| | | | Potential Phosphorylation Sites: S50 S100 S103 S188 S272 S528 T193 T471 T491 T498 | MOTIFS |
| | | | T503 T547 T598 T599 T616 Y232 Y480 | |
| 10 | 7513162CD1 | 1433 | signal_cleavage: M1-G19 | SPSCAN |
| | | | Signal Peptide: M1-G19 | HMMER |
| | | | Signal Peptide: M1-P21 | HMMER |
| | | | Signal Peptide: M1-Q22 | HMMER |
| | | | Signal Peptide: M1-T25 | HMMER |
| | | | Lipase/Acylhydrolase with GDSL-like motif: V740-D868, V393-D521, V1096-N1219 | HMMER_PFAM |
| | | | Cytosolic domain: W1413-L1433 | TMHMMER |
| | | | Transmembrane domain: V1390-V1412 | |
| | | | Non-cytosolic domain: M1-E1389 | |
| | | | GDSL lipolytic enzyme IPB001087: I394-G404 | BLIMPS_BLOCKS |
| | | | PHOSPHOLIPASE B ADRABB PRECURSOR HYDROLASE REPEAT SIGNAL | BLAST_PRODOM |
| | | | TRANSMEMBRANE PD024730: F1071-L1224, I23-V199, K355-C519 | |
| | | | PHOSPHOLIPASE B ADRABB PRECURSOR HYDROLASE REPEAT SIGNAL | BLAST_PRODOM |
| | | | TRANSMEMBRANE PD152478: N1055-V1096 | |
| | | | PHOSPHOLIPASE B ADRABB PRECURSOR PROTEIN HYDROLASE REPEAT SIGNAL | BLAST_PRODOM |
| | | | TRANSMEMBRANE F09C8.1 PD003965: D194-S347, Q528-S707 N1365-R1378, | |
| | | | F877-S1054, E1218-P1369 | |
| | | | SIMILAR TO PHOSPHOLIPASE ADRABB PRECURSOR PD134752: D358-L538, | BLAST_PRODOM |
| | | | D1070-N1219, S729-F877 | |
| | | | ADRAB-B; PHOSPHOLIPASE; DM03287\|Q05017\|713-1063: P47-N78, T713-I1064, | BLAST_DOMO |
| | | | C370-S707, C1073-P1369, M107-S301, L196-Y349 | |
| | | | ADRAB-B; PHOSPHOLIPASE; DM03287\|Q05017\|360-711: L360-G712, | BLAST_DOMO |
| | | | E1171-S1192, G712-S1054, G1068-P1369, E39-V121, M104-F284 | |
| | | | ADRAB-B; PHOSPHOLIPASE; DM03287\|Q05017\|1065-1411: E1065-P1369, | BLAST_DOMO |
| | | | E1367-E1385, G365-S707, P727-S1054, S44-H292 | |
| | | | ADRAB-B; PHOSPHOLIPASE; DM03287\|Q05017\|41-358: L41-K359, F1071-S1377, | BLAST_DOMO |
| | | | C370-S450, V819-L986, P716-D763, I434-F631, L934-S1054 | |
| | | | Potential Phosphorylation Sites: S26 S30 S64 S256 S267 S271 S324 S343 S450 S614 | MOTIFS |
| | | | S657 S756 S954 S961 S1025 S1121 S1158 S1284 S1351 S1427 T31 T40 T96 T128 | |
| | | | T245 T458 T554 T596 T619 T680 T703 T933 T966 T1042 T1050 T1312 T1373 | |
| | | | Potential Glycosylation Sites: N173 N240 N493 N529 N590 N690 N783 N797 N809 | MOTIFS |
| | | | N1055 N1113 N1114 N1275 | |
| | | | Lipolytic enzymes “G-D-S-L” family, serine active site: I394-G404, V741-G751 | MOTIFS |
| 11 | 7513164CD1 | 1004 | signal_cleavage: M1-G19 | SPSCAN |
| | | | Signal Peptide: M1-G19 | HMMER |
| | | | Signal Peptide: M1-P21 | HMMER |
| | | | Signal Peptide: M1-Q22 | HMMER |
| | | | Signal Peptide: M1-T25 | HMMER |
| | | | Lipase/Acylhydrolase with GDSL-like motif: V740-D868, V393-D521 | HMMER_PFAM |
| | | | GDSL lipolytic enzyme IPB001087: I394-G404, G511-D521, D652-S657 | BLIMPS_BLOCKS |
| | | | PHOSPHOLIPASE B ADRABB PRECURSOR HYDROLASE REPEAT SIGNAL | BLAST_PRODOM |
| | | | TRANSMEMBRANE PD024730: I23-V199, K355-C519 | |
| | | | PHOSPHOLIPASE B ADRABB PRECURSOR HYDROLASE REPEAT SIGNAL | BLAST_PRODOM |
| | | | TRANSMEMBRANE PD152479: T351-V393 | |
| | | | PHOSPHOLIPASE B ADRABB PRECURSOR PROTEIN HYDROLASE REPEAT SIGNAL | BLAST_PRODOM |
| | | | TRANSMEMBRANE F09C8.1 PD003965: D194-S347, Q528-S707 N1365-R1378, | |
| | | | F877-S1054, E1218-P1369 | |
| | | | SIMILAR TO PHOSPHOLIPASE ADRABB PRECURSOR PD134752: D358-L538, | BLAST_PRODOM |
| | | | S729-F877 | |
| | | | ADRAB-B; PHOSPHOLIPASE; DM03287\|Q05017\|360-711: L360-G712, G712-C928, | BLAST_DOMO |
| | | | E39-V121, M104-F284 | |
| | | | ADRAB-B; PHOSPHOLIPASE; DM03287\|Q05017\|41-358: L41-K359, C370-S450, | BLAST_DOMO |
| | | | I434-F631, F604-Q709, V819-D901 | |
| | | | ADRAB-B; PHOSPHOLIPASE; DM03287\|Q05017\|713-1063: P47-N78, C370-S707, | BLAST_DOMO |
| | | | T713-C928, M107-S301, L196-Y349 | |
| | | | ADRAB-B; PHOSPHOLIPASE; DM03287\|Q05017\|1065-1411: G365-S707, P727-L903, | BLAST_DOMO |
| | | | S44-H292 | |
| | | | Potential Phosphorylation Sites: S26 S30 S64 S256 S267 S271 S324 S343 S450 S614 | MOTIFS |
| | | | S657 S756 S943 S950 S958 S983 T31 T40 T96 T128 T245 T458 T554 T596 | |
| | | | T619 T680 T703 T938 T942 | |
| | | | Potential Glycosylation Sites: N173 N240 N493 N529 N590 N690 N783 N797 N809 | MOTIFS |
| | | | Lipolytic enzymes “G-D-S-L” family, serine active site: I394-G404, V741-G751 | MOTIFS |
| 12 | 7513496CD1 | 380 | Signal Peptide: M1-P21 | HMMER |
| | | | Signal Peptide: M1-G23 | HMMER |
| | | | APOLIPOPROTEIN L PRECURSOR APOL PLASMA LIPID TRANSPORT | BLAST_PRODOM |
| | | | GLYCOPROTEIN SIGNAL DJ68O2.1 PD042084: V16-L380 | |
| | | | Potential Phosphorylation Sites: S22 S131 S208 S307 T71 T349 | MOTIFS |
| | | | Potential Glycosylation Sites: N243 | MOTIFS |
| 13 | 7514724CD1 | 99 | signal_cleavage: M1-S19 | SPSCAN |
| | | | Signal Peptide: M1-G15 | HMMER |
| | | | Signal Peptide: M1-S19 | HMMER |
| | | | Collagen triple helix repeat (20 copies): R24-V82 | HMMER_PFAM |
| | | | PRECOLLAGEN P PRECURSOR SIGNAL PD072959: G15-G89 | BLAST_PRODOM |
| | | | MANNOSE-BINDING LECTIN DM01663\|P08427\|1-117: L8-G89 | BLAST_DOMO |
| | | | MANNOSE-BINDING LECTIN DM01663\|P06908\|1-117: M1-M90 | BLAST_DOMO |
| | | | MANNOSE-BINDING LECTIN DM01663\|P12842\|1-116: L6-M90 | BLAST_DOMO |
| | | | MANNOSE-BINDING LECTIN DM01663\|P35242\|1-117: L8-G89 | BLAST_DOMO |
| | | | Potential Phosphorylation Sites: S96 T22 T69 | MOTIFS |
| 14 | 7514797CD1 | 304 | signal_cleavage: M1-A38 | SPSCAN |
| | | | Enoyl-CoA hydratase/isomerase family: L68-Q249 | HMMER_PFAM |
| | | | Enoyl-CoA hydratase/isomerase IPB001753: V70-M81, R103-S125, K161-C187, | BLIMPS_BLOCKS |
| | | | T208-A247 | |
| | | | Enoyl-CoA hydratase/isomerase signature: Q150-A204 | PROFILESCAN |
| | | | ENOYL-COA PROBABLE PEROXISOMAL HYDRATASE FATTY ACID METABOLISM | BLAST_PRODOM |
| | | | LYASE PEROXISOME SIMILAR PD015471: K226-L304 | |
| | | | PROTEIN HYDRATASE ENOYL-COA ACID FATTY LYASE ISOMERASE | BLAST_PRODOM |
| | | | METABOLISM 3-HYDROXY-ACYL-COA DEHYDROGENASE PD000432: | |
| | | | V70-G217 | |
| | | | PROBABLE PEROXISOMAL ENOYL-COA HYDRATASE FATTY ACID METABOLISM | BLAST_PRODOM |
| | | | LYASE PEROXISOME PD029838: G21-H69 | |
| | | | ENOYL-COA HYDRATASE/ISOMERASE DM00366\|I38882\|54-320: S54-L234 | BLAST_DOMO |
| | | | G217-K297 | |
| | | | ENOYL-COA HYDRATASE/ISOMERASE DM00366\|A57626\|53-319: S54-L234 | BLAST_DOMO |
| | | | G217-K297 | |
| | | | ENOYL-COA HYDRATASE/ISOMERASE DM00366\|P31551\|36-292: E56-I216 | BLAST_DOMO |
| | | | G217-E295 | |
| | | | ENOYL-COA HYDRATASE/ISOMERASE DM00366\|P52046\|1-255: V70-T251 | BLAST_DOMO |
| | | | Potential Phosphorylation Sites: S8 S30 S37 S57 S241 S250 S262 T16 T20 T180 | MOTIFS |
| | | | Potential Glycosylation Sites: N218 N274 | MOTIFS |
| | | | Enoyl-CoA hydratase/isomerase signature: I164-I184 | MOTIFS |
| 15 | 7512100CD1 | 180 | PhoX homologous domain, present in p47phox and p40phox: D76-H176 | HMMER_SMART |
| | | | PX domain: D76-H176 | HMMER_PFAM |
| | | | Neutrophil cytosol factor P40 signature | BLIMPS_PRINTS |
| | | | PR00497: F113-F130 | |
| | | | PROTEIN PHOSPHOLIPASE 3-KINASE D SORTING NEXIN D2 CHROMOSOME | BLAST_PRODOM |
| | | | PHOSPHOINOSITIDE P47PHOX | |
| | | | PD003685: K94-H176 (P = 5.3e−09) | |
| | | | Potential Phosphorylation Sites: | MOTIFS |
| | | | S39, S53, S58, S177, T21, T105, T117 | |
| | | | Potential Glycosylation Sites: | MOTIFS |
| | | | N38 | |
| 16 | 7512101CD1 | 209 | PhoX homologous domain, present in p47phox and p40phox: D105-H205 | HMMER_SMART |
| | | | PX domain: D105-H205 | HMMER_PFAM |
| | | | Neutrophil cytosol factor P40 signature | BLIMPS_PRINTS |
| | | | PR00497: F142-F159 | |
| | | | PROTEIN PHOSPHOLIPASE 3-KINASE D SORTING NEXIN D2 CHROMOSOME | BLAST_PRODOM |
| | | | PHOSPHOINOSITIDE P47PHOX | |
| | | | PD003685: K123-H205 (P = 5.3e−09) | |
| | | | Potential Phosphorylation Sites: | MOTIFS |
| | | | S39, S56, S82, S87, S206, T21, T134, T146 | |
| | | | Potential Glycosylation Sites: | MOTIFS |
| | | | N38, N63 | |
| 17 | 7516771CD1 | 419 | signal_cleavage: M1-S19 | SPSCAN |
| | | | Signal Peptide: M1-S19, M1-G21, M1-G24, M1-A28, M3-H18, M3-S19, M3-G24 | HMMER |
| | | | alpha/beta hydrolase fold: F133-I412 | HMMER_PFAM |
| | | | Lipase | BLIMPS_BLOCKS |
| | | | IPB000734: E186-G200 | |
| | | | LIPASE HYDROLASE PRECURSOR SIGNAL LIPID DEGRADATION PROTEIN | BLAST_PRODOM |
| | | | GLYCOPROTEIN ESTERASE TRIACYLGLYCEROL | |
| | | | PD003556: A28-M415 | |
| | | | TRIACYLGLYCEROL LIPASE, LINGUAL | BLAST_DOMO |
| | | | DM02342\|P38571\|3-397: M3-G77, G97-Y418 | |
| | | | DM02342\|P07098\|35-395: V37-V81, G97-M415 | |
| | | | DM02342\|P04634\|32-394: M35-V81, G97-M415 | |
| | | | DM02342\|JC4017\|1-394: M3-V81, G97-M415 | |
| | | | Potential Phosphorylation Sites: | MOTIFS |
| | | | S74, S89, S146, S155, S163, S295, S333, S388, T27, T183, Y189 | |
| | | | Potential Glycosylation Sites: | MOTIFS |
| | | | N36, N72, N121, N181, N293, N341 | |
| | | | Lipases, serine active site: | MOTIFS |
| | | | V188-T197 | |
| 18 | 7512128CD1 | 244 | CDP-alcohol phosphatidyltransferase: G94-F242 | HMMER_PFAM |
| | | | Cytosolic domains: M1-T46, D104-E122, G173-G178, R244-R244 | TMHMMER |
| | | | Transmembrane domains: W47-A69, H84-L103, L123-G145, G150-W172, I179-A201, | |
| | | | L221-F243 | |
| | | | Non-cytosolic domains: Y70-K83, R146-T149, V202-D220 | |
| | | | CDP-alcohol phosphatidyltransferase IPB000462: D104-D129 | BLIMPS_BLOCKS |
| | | | CDP-alcohol phosphatidyltransferases signature: D87-T149 | PROFILESCAN |
| | | | TRANSFERASE AMINOALCOHOLPHOSPHOTRANSFERASE PHOSPHOLIPID | BLAST_PRODOM |
| | | | BIOSYNTHESIS MEMBRANE MICROSOME TRANSMEMBRANE PROTEIN | |
| | | | CHOLINEPHOSPHOTRANSFERASE SN1 PD008780: G3-V233 | |
| | | | CDP-ALCOHOL PHOSPHATIDYLTRANSFERASES | BLAST_DOMO |
| | | | DM07601\|P22140\|1-390: G3-D220 | |
| | | | Potential Phosphorylation Sites: S21 | MOTIFS |
| | | | Potential Glycosylation Sites: N115 | MOTIFS |
| | | | CDP-alcohol phosphatidyltransferases signature: D107-D129 | MOTIFS |
| 19 | 7518098CD1 | 158 | Cytosolic domains: M1-E70, R121-P158 | TMHMMER |
| | | | Transmembrane domains: Y71-I93, F98-H120 | |
| | | | Non-cytosolic domain: P94-K97 | |
| | | | Fatty acid desaturase, type 1 IPB001522: T15-P24, K62-V105, F106-R135 | BLIMPS_BLOCKS |
| | | | Fatty acid desaturase family 1 signature PR00075: W73-I93, K97-A119, H120-I140 | BLIMPS_PRINTS |
| | | | DESATURASE ACID FATTY ACYL-COA STEAROYL-COA OXIDOREDUCTASE | BLAST_PRODOM |
| | | | DELTA9-DESATURASE IRON BIOSYNTHESIS RETICULUM | |
| | | | PD002221: 176-Q147 | |
| | | | DESATURASE ACID FATTY ACYL-COA STEAROYL-COA OXIDOREDUCTASE | BLAST_PRODOM |
| | | | DELTA9-DESATURASE IRON BIOSYNTHESIS ENDOPLASMIC | |
| | | | PD013924: P23-I76 | |
| | | | STEAROYL-COA DESATURASE | BLAST_DOMO |
| | | | DM02647\|JX0150\|58-343: Y59-S148 | |
| | | | DM02647\|P13516\|55-340: Y59-S148 | |
| | | | DM02647\|S52746\|37-342: W73-L138 | |
| | | | Potential Phosphorylation Sites: S66, S124, S127, T58, T95 | MOTIFS |
| 20 | 7524729CD1 | 426 | Signal Peptide: M1-S21, M1-P22, M1-G20, M1-A18 | HMMER |
| | | | signal_cleavage: M1-A18 | SPSCAN |
| | | | PLAT/LH2 domain: Y273-C409 | HMMER_PFAM |
| | | | Lipase: S21-F270 | HMMER_PFAM |
| | | | Lipoxygenase homology 2 (beta barrel) domain: Y273-C409 | HMMER_SMART |
| | | | Lipase IPB000734: G161-A175 | BLIMPS_BLOCKS |
| | | | Lipases, serine active site: H143-I192 | PROFILESCAN |
| | | | Triacylglycerol lipase family signature PR00821: P73-W92, S95-L109, V119-A134, | BLIMPS_PRINTS |
| | | | R139-F158, N162-N180, C198-N213, F229-G246, | |
| | | | P269-Y284, T321-W342 | |
| | | | Lipoprotein lipase signature PR00822: N52-G69, T93-N117, G176-R188 | BLIMPS_PRINTS |
| | | | LIPASE PRECURSOR SIGNAL HYDROLASE DEGRADATION LIPID PANCREATIC | BLAST_PRODOM |
| | | | GLYCOPROTEIN LIPOPROTEIN YOLK PD001492: R50-I192 L160-C409 | |
| | | | TRIACYLGLYCEROL LIPASE | BLAST_DOMO |
| | | | DM00344\|P11602\|27-345: R50-I192 I192-K278 | |
| | | | TRIACYLGLYCEROL LIPASE | BLAST_DOMO |
| | | | DM00344\|P11153\|17-335: R50-I192 E194-K278 | |
| | | | TRIACYLGLYCEROL LIPASE | BLAST_DOMO |
| | | | DM00344\|S15893\|37-357: K39-I192 I189-K278 | |
| | | | TRIACYLGLYCEROL LIPASE | BLAST_DOMO |
| | | | DM00344\|P27656\|37-357: K39-I192 I189-K278 | |
| | | | Potential Phosphorylation Sites: S48, S226, S236, S258, S283, T41, T55, T82, T301, | MOTIFS |
| | | | T328, T382, T387 | |
| | | | Potential Glycosylation Sites: N80, N136, N319, N395, N417 | MOTIFS |
| | | | Lipases, serine active site: V163-A172 | MOTIFS |
| 21 | 7520475CD1 | 909 | C2 domain: C590-I673 | HMMER_PFAM |
| | | | Phosphatidylinositol-specific phospholipase C, X domain: E201-R351 | HMMER_PFAM |
| | | | Phosphatidylinositol-specific phospholipase C, Y domain: H451-R568 | HMMER_PFAM |
| | | | Protein kinase C conserved region 2 (CalB): T589-L688 | HMMER_SMART |
| | | | Phospholipase C, catalytic domain (part); domain X: Q200-K350 | HMMER_SMART |
| | | | Phospholipase C, catalytic domain (part); domain Y: L452-R568 | HMMER_SMART |
| | | | Phospholipase C signature PR00390: P205-Q223, E231-G251, A334-R351, | BLIMPS_PRINTS |
| | | | M506-W527, W527-M545, L674-R684 | |
| | | | PHOSPHOLIPASE PHOSPHODIESTERASE C HYDROLASE 1- | BLAST_PRODOM |
| | | | PHOSPHATIDYLINOSITOL-4,5-BISPHOSPHATE LIPID DEGRADATION | |
| | | | TRANSDUCER PHOSPHOINOSITIDE-SPECIFIC PD001202: | |
| | | | L452-R568 | |
| | | | PHOSPHOLIPASE C PHOSPHODIESTERASE HYDROLASE 1- | BLAST_PRODOM |
| | | | PHOSPHATIDYLINOSITOL-4,5-BISPHOSPHATE LIPID DEGRADATION | |
| | | | TRANSDUCER PHOSPHOINOSITIDE-SPECIFIC PD001214: | |
| | | | E201-R351 | |
| | | | PHOSPHOLIPASE BETA C HYDROLASE 1-PHOSPHATIDYLINOSITOL-4,5- | BLAST_PRODOM |
| | | | BISPHOSPHATE PHOSPHODIESTERASE LIPID DEGRADATION | |
| | | | TRANSDUCER PD005847: S55-A155 | |
| | | | BETA PHOSPHOLIPASE 1-PHOSPHATIDYLINOSITOL-4,5-BISPHOSPHATE | BLAST_PRODOM |
| | | | PHOSPHODIESTERASE PLC154 HYDROLASE LIPID DEGRADATION TRANSDUCER | |
| | | | PD023749: E355-H451 | |
| | | | 1-PHOSPHATIDYLINOSITOL-4,5-BISPHOSPHATE PHOSPHODIESTERASE D | BLAST_DOMO |
| | | | DM00712\|A48047\|523-820: A409-L707 | |
| | | | DM00712\|A53766\|83-369: Y441-L707 | |
| | | | 1-PHOSPHATIDYLINOSITOL-4,5-BISPHOSPHATE PHOSPHODIESTERASE D | BLAST_DOMO |
| | | | DM00855\|A48047\|58-521: M1-S408 | |
| | | | DM00855\|P13217\|63-512: M1-A397 | |
| | | | Potential Phosphorylation Sites: S62, S80, S228, S337, S366, S408, S421, S438, S481, | MOTIFS |
| | | | S601, S687, S730, S757, S776, S790, S828, S860, T65, T100, T107, T220, T428, | |
| | | | T580, T607, T620, T732, T756, T767, T846, T885, Y123 | |
| | | | Potential Glycosylation Sites: N478, N483, N555, N904 | MOTIFS |
| | | | ATP/GTP-binding site motif A (P-loop): G221-S228 | MOTIFS |

TABLE 4

| Polynucleotide SEQ ID NO:/ | |
|---|---|
| Incyte ID/Sequence Length | Sequence Fragments |
| 22/7511098CB1/645 | 1-258, 1-418, 1-645, 222-645 |
| 23/7522037CB1/287 | 1-180, 1-287, 2-286, 180-287 |
| 24/7524271CB1/1159 | 1-769, 196-442, 196-642, 203-379, 203-463, 245-441, 260-506, 260-629, 271-549, 295-540, 298-571, 298-581, 332-597, 339-503, 368-574, |
| | 397-619, 416-632, 428-666, 439-650, 439-967, 470-733, 483-886, 495-694, 515-798, 627-1159, 640-854, 656-774, 658-923, 667-858, |
| | 691-950, 698-916, 698-920, 711-882, 711-922, 724-1120, 725-798, 729-1008, 729-1012, 730-982, 730-993, 730-995, 731-1019 |
| 25/7513132CB1/4568 | 1-587, 11-4562, 35-592, 106-367, 125-379, 215-512, 295-745, 357-569, 418-518, 438-534, 480-1073, 549-1038, 550-862, 736-1465, |
| | 805-1276, 917-1652, 917-1697, 1019-1677, 1050-1541, 1194-1666, 1228-1834, 1236-1574, 1293-1984, 1303-1824, 1365-1758, 1407-1944, |
| | 1419-1796, 1428-1632, 1480-2035, 1504-2051, 1523-2241, 1592-2045, 1595-2049, 1675-2105, 1677-2192, 1680-1857, 1680-1930, |
| | 1711-2401, 1711-2522, 1719-2522, 1720-2306, 1758-2522, 1759-2164, 1807-2112, 1807-2409, 1846-2120, 1862-2284, 1872-2542, |
| | 1895-2484, 1902-2491, 1910-2729, 1930-2504, 1985-2551, 1999-2492, 2011-2642, 2031-2302, 2054-2633, 2099-2665, 2138-2701, |
| | 2148-2355, 2495-2751, 2501-2729, 2517-2692, 2623-2877, 2728-3137, 2731-3234, 2773-3065, 2784-3019, 2826-3142, 2837-2956, |
| | 2864-3095, 2872-3115, 2873-3218, 2885-3600, 2935-3642, 2944-3201, 2945-3513, 3002-3260, 3037-3124, 3050-3235, 3095-3658, |
| | 3135-3798, 3147-3674, 3156-3433, 3160-3405, 3192-3960, 3245-3484, 3246-3522, 3246-3639, 3258-3520, 3265-3560, 3314-3571, |
| | 3314-3898, 3321-3899, 3344-3550, |
| | 3395-3650, 3423-3644, 3430-3836, 3431-3994, 3455-3575, 3497-3713, 3497-4108, 3536-3804, 3540-4100, 3554-3821, 3567-3824, |
| | 3580-3820, 3580-4227, 3626-3934, 3627-3912, 3672-3913, 3729-4561, 3732-4408, 3756-3999, 3765-4368, 3776-4290, 3792-4039, |
| | 3821-4454, 3831-4411, 3841-4122, 3842-4066, 3842-4280, 3851-4364, 3857-3937, 3857-4083, 3857-4099, 3859-4422, 3874-4145, |
| | 3895-4470, 3921-4180, 3924-4488, 3927-4188, 3939-4517, 3945-4419, 3965-4191, 3968-4568, 3984-4460, 4000-4562, 4093-4312, |
| | 4097-4310, 4103-4376, 4113-4521, 4132-4426, 4167-4342, 4183-4562, 4228-4465, 4245-4461, 4251-4503, 4319-4556, 4332-4440, |
| | 4369-4449 |
| 26/7513134CB1/4435 | 1-587, 11-4425, 35-592, 106-367, 125-379, 215-512, 295-745, 357-569, 418-518, 438-534, 480-1073, 549-1038, 550-862, 736-1465, |
| | 805-1276, 917-1644, 917-1652, 917-1697, 1019-1677, 1050-1541, 1194-1666, 1228-1834, 1236-1574, 1293-1984, 1303-1824, 1365-1758, |
| | 1407-1944, 1419-1796, 1428-1632, 1480-2035, 1504-2051, 1523-2241, 1592-2045, 1595-2049, 1675-2105, 1677-2192, 1680-1857, |
| | 1680-1930, 1711-2401, 1711-2522, 1719-2522, 1720-2306, 1758-2522, 1759-2164, 1807-2112, 1807-2409, 1846-2120, 1862-2284, |
| | 1872-2542, 1895-2484, 1902-2491, 1910-2730, 1930-2504, 1985-2551, 1999-2492, 2011-2642, 2031-2302, 2054-2633, 2099-2665, |
| | 2138-2701, 2148-2355, 2310-3043, 2366-2901, 2394-3043, 2420-2976, 2485-3019, 2495-2729, 2496-2522, 2501-2759, 2517-2692, |
| | 2562-3205, 2564-2792, 2568-2919, 2577-3079, 2615-3094, 2631-2871, 2632-2831, 2650-3069, 2703-2963, 2706-3025, 2733-3390, |
| | 2750-3186, 2770-3283, 2777-2800, 2822-3114, 2833-3068, 2869-3206, 2875-3191, 2886-3005, 2913-3144, 2921-3164, 2922-3267, 2934-3649, 2984-3691, 2993-3250, 2994-3562, |
| | 3051-3309, 3086-3173, 3099-3284, 3144-3707, 3196-3724, 3205-3482, 3209-3454, 3294-3533, 3295-3571, 3295-3688, 3307-3569, |
| | 3314-3609, 3363-3620, 3393-3599, 3444-3699, 3461-3691, 3472-3693, 3504-3624, 3504-3691, 3576-3691, 3589-4224, 3721-3798, |
| | 3721-3927, 3721-3944, 3721-3960, 3721-3983, 3721-4141, 3721-4225, 3721-4283, 3721-4315, 3735-4006, 3756-4331, 3782-4041, |
| | 3785-4349, 3788-4049, 3800-4378, 3806-4280, 3826-4052, 3829-4423, 3845-4321, 3861-4425, 3954-4173, 3958-4171, 3964-4237, |
| | 3974-4382, 3993-4287, 4028-4203, 4044-4435, 4089-4326, 4106-4322, 4112-4364, 4180-4417, 4193-4301, 4230-4310 |
| 27/7523653CB1/1357 | 1-649, 1-685, 1-702, 1-804, 2-1356, 634-1356, 634-1357, 639-1357 |
| 28/7751418CB1/3703 | 1-679, 228-773, 230-460, 230-733, 230-871, 230-889, 230-3691, 230-3703, 324-705, 363-529, 363-531, 424-531, 507-929, 550-872, |
| | 550-994, 550-1072, 553-1196, 575-1032, 577-1164, 600-705, 617-861, 617-1252, 622-1297, 626-1002, 636-1328, 650-828, 700-1327, |
| | 703-881, 704-881, 721-881, 721-1311, 823-1291, 823-1437, 824-1477, 831-1477, 850-1282, 873-1184, 879-1046, 894-1414, 1116-1752, |
| | 1141-1788, 1166-1788, 1171-1788, 1190-1404, 1202-1816, 1206-1516, 1224-1540, 1301-1788, 1322-1351, 1331-1590, 1331-1730, |
| | 1438-1929, 1451-1995, 1514-2004, 1514-2124, 1545-1733, 1545-1737, 1545-1759, 1545-2207, 1545-2274, 1553-2205, 1623-2274, |
| | 1623-2275, 1649-2191, 1658-2207, 1662-1961, 1691-2207, 1727-2274, 1744-1962, 1788-2032, 1788-2207, 1826-2180, 1836-2452, |
| | 1839-2091, 1839-2327, 1867-2207, 1954-2274, 1961-2274, 2014-2049, 2014-2086, 2014-2161, 2014-2274, 2016-2274, 2023-2526, |
| | 2061-2542, 2079-2366, 2085-2166, 2085-2349, 2151-2714, 2166-2273, 2166-2316, 2192-2591, 2225-2718, 2270-2366, 2277-2316, 2299-2526, 2316-2361, 2316-2396, |
| | 2316-2413, 2316-2526, 2324-2366, 2343-2948, 2369-2607, 2369-2799, 2369-2802, 2415-2526, 2435-3278, 2526-2557, 2526-2681, |
| | 2526-2926, 2547-3145, 2561-2780, 2596-3190, 2603-2869, 2603-2900, 2641-3238, 2717-3432, 2760-3335, 2765-3400, 2780-3288, |
| | 2809-2965, 2809-3073, 2859-3362, 2878-3515, 2939-3217, 2957-3473, 2988-3477, 2989-3640, 2995-3591, 3033-3583, 3035-3317, |
| | 3062-3117, 3062-3154, 3062-3310, 3062-3388, 3062-3450, 3068-3315, 3073-3703, 3093-3703, 3122-3355, 3122-3368, 3136-3368, |
| | 3137-3359, 3146-3368, 3227-3691, 3227-3693, 3227-3698, 3248-3369, 3250-3368, 3268-3703, 3298-3703, 3302-3368, 3322-3703, |
| | 3330-3566, 3345-3703, 3397-3703, 3458-3703, 3462-3703, 3479-3703, 3480-3703, 3493-3703, 3502-3703, 3513-3703, 3522-3703, |
| | 3524-3703, 3568-3703, 3599-3703, 3601-3703, 3662-3703, 3666-3703 |
| 29/7523952CB1/1704 | 1-598, 2-1703, 352-1238, 376-1242, 553-1459, 553-1469, 1037-1703, 1139-1704 |
| 30/7513020CB1/2388 | 1-217, 1-369, 1-423, 1-457, 1-462, 1-469, 1-587, 1-600, 1-603, 1-618, 1-652, 1-656, 1-663, 1-719, 1-834, 1-2388, 2-303, 3-193, 4-208, |
| | 5-221, 5-233, 6-233, 7-233, 9-799, 18-613, 40-186, 233-477, 233-491, 233-666, 238-771, 241-898, 249-466, 256-692, 311-579, 312-907, |
| | 312-910, 312-934, 343-1088, 355-930, 386-652, 428-668, 431-938, 440-1092, 451-726, 451-954, 470-742, 506-1069, 539-833, |
| | 539-1051, 554-798, 554-806, 576-1127, 582-1084, 590-1163, 600-885, 607-1147, 617-1262, 623-1232, 641-929, 684-963, 712-1234, |
| | 767-1041, 768-928, 772-1298, 786-1338, 788-1338, 789-1048, 789-1585, 809-1370, 821-1473, 826-1424, 837-1097, 840-1055, 841-1069, |
| | 850-1324, 860-1091, 863-1071, 864-1065, 864-1141, 864-1383, 865-1082, 865-1646, 873-1161, 884-1124, 884-1159, 889-1271, |
| | 893-1483, 910-1452, 910-1480, 914-1456, 914-1504, 921-1217, 925-1467, 926-1173, 928-1567, 941-1214, 943-1391, 955-1624, 970-1224, |
| | 970-1464, 987-1101, 996-1236, 997-1235, 998-1612, 1016-1438, 1021-1620, 1033-1581, 1034-1592, 1055-1416, 1060-1326, |
| | 1065-1656, |
| | 1072-1681, 1074-1328, 1074-1359, 1075-1624, 1085-1555, 1086-1273, 1088-1716, 1094-1771, 1098-1705, 1101-1417, 1105-1325, |
| | 1106-1704, 1107-1251, 1107-1479, 1115-1539, 1115-1716, 1116-1705, 1121-1371, 1126-1751, 1129-1607, 1131-1580, 1132-1399, |
| | 1136-1688, 1140-1688, 1143-1708, 1145-1338, 1155-1508, 1166-1861, 1172-1669, 1183-1774, 1187-1749, 1187-1794, 1189-1882, |
| | 1190-1587, 1206-1820, 1238-1474, 1247-1903, 1250-1748, 1253-1898, 1254-1633, 1272-1776, 1273-1657, 1278-1486, 1293-1883, |
| | 1298-1867, 1301-1910, 1305-1773, 1308-1513, 1308-1910, 1312-1897, 1318-1902, 1322-1614, 1323-1573, 1324-1772, 1335-1649, |
| | 1336-1525, 1348-1610, 1348-1715, 1352-1715, 1352-1880, 1359-1952, 1362-1981, 1383-1993, 1399-1905, 1401-1997, 1411-1998, |
| | 1428-1629, 1430-1594, 1433-1655, 1437-2206, 1445-1999, 1446-1675, 1448-1670, 1462-1999, 1473-1620, 1484-1898, 1485-2020, |
| | 1485-2191, 1490-1805, 1494-2088, 1495-1749, 1496-1993, 1500-2076, 1521-2217, 1530-1817, 1544-1763, 1544-2152, 1547-2072, |
| | 1555-2058, 1556-1815, 1556-2226, 1560-2379, 1567-1857, |
| | 1590-2168, 1598-1844, 1614-1875, 1623-1914, 1630-1869, 1630-1893, 1635-2120, 1637-2158, 1661-2213, 1673-1960, 1680-2037, |
| | 1684-1900, 1686-1899, 1686-1943, 1686-2169, 1688-1817, 1688-1954, 1689-1924, 1691-2265, 1696-1942, 1708-2213, 1711-2204, |
| | 1711-2216, 1716-1985, 1718-2256, 1728-2261, 1730-1978, 1732-2210, 1733-2270, 1744-1995, 1744-2134, 1746-1975, 1746-1994, |
| | 1746-2031, 1748-2261, 1772-2035, 1785-1971, 1801-2259, 1802-2259, 1808-2068, 1809-2261, 1809-2270, 1812-2262, 1814-2261, |
| | 1820-2202, 1820-2232, 1820-2259, 1826-2270, 1828-2266, 1830-2261, 1831-2270, 1833-2270, 1836-1934, 1838-2075, 1838-2150, |
| | 1845-2100, 1849-2261, 1852-2261, 1855-2261, 1855-2275, 1858-2266, 1858-2270, 1863-2087, 1863-2261, 1872-2261, 1876-2244, |
| | 1879-2261, 1881-2208, 1881-2260, 1883-2261, 1884-2266, 1887-2260, 1888-2267, 1889-2261, 1890-2268, 1895-2266, 1897-2263, |
| | 1905-2266, 1910-2261, 1915-2265, 1919-2275, 1927-2265, 1939-2261, 1954-2361, 1957-2275, 1958-2388, 1961-2261, 1971-2100, |
| | 1971-2230, 1974-2261, 1984-2266, 1995-2266, 1998-2261, |
| | 1999-2208, 1999-2266, 2004-2124, 2004-2261, 2005-2259, 2009-2266, 2011-2266, 2014-2258, 2015-2266, 2023-2266, 2027-2261, |
| | 2033-2261, 2034-2267, 2034-2270, 2038-2262, 2040-2266, 2043-2261, 2048-2388, 2067-2270, 2074-2266, 2082-2261, 2133-2333, |
| | 2164-2361, 2169-2275, 2186-2262, 2186-2266 |
| 31/7513162CB1/4508 | 1-412, 1-4508, 105-505, 270-662, 538-1036, 538-1077, 538-1203, 538-1211, 538-1218, 538-1234, 540-1216, 540-1294, 542-1211, |
| | 620-1067, 620-1173, 620-1191, 620-1212, 620-1216, 620-1235, 620-1291, 620-1300, 620-1307, 620-1311, 620-1340, 620-1414, 620-1430, |
| | 622-1245, 622-1253, 622-1337, 631-1294, 692-1430, 707-1430, 763-1430, 778-1430, 779-1430, 780-1430, 798-1430, 801-1430, |
| | 813-1430, 822-1430, 853-1421, 853-1430, 862-1430, 867-1430, 874-1430, 895-1430, 899-1430, 909-1430, 923-1430, 924-1430, 926-1430, |
| | 932-1430, 936-1430, 968-1430, 992-1430, 1002-1430, 1006-1430, 1023-1430, 1028-1430, 1042-1430, 1045-1430, 1390-1663, |
| | 2163-2694, 2389-3021, 2621-2874, 2644-3087, 2670-3186, 2738-3334, 3034-3664, 3061-3753, 3077-3716, 3083-3563, 3113-3724, |
| | 3126-3818, 3162-3888, 3173-4006, 3176-3277, 3176-3472, 3176-3700, 3176-3738, 3188-3464, 3188-3585, 3188-3648, 3188-3664, |
| | 3188-3701, 3188-3706, 3188-3740, 3188-3789, 3188-3807, 3188-3839, 3192-3550, 3231-3768, 3254-3917, 3266-4046, 3269-3807, |
| | 3280-3883, 3305-4013, 3325-3901, |
| | 3336-3864, 3345-3892, 3352-3871, 3358-3930, 3405-4083, 3415-4072, 3442-4074, 3512-3965, 3554-3812, 3582-3839, 3582-3895, |
| | 3609-4052, 3644-4106, 3673-4254, 3676-3940, 3703-4105, 3708-4060, 3711-3976, 3728-4113, 3734-3923, 3765-3988, 3765-4023, |
| | 3781-4441, 3810-4106, 3860-4468, 3866-4078, 3914-4262, 4107-4163, 4107-4350, 4107-4474, 4107-4508, 4135-4508, 4136-4453, |
| | 4155-4508, 4189-4508, 4193-4455, 4193-4464, 4328-4508 |
| 32/7513164CB1/4512 | 1-412, 1-4512, 105-505, 270-662, 538-1036, 538-1077, 538-1203, 538-1211, 538-1218, 538-1234, 540-1216, 540-1294, 542-1211, |
| | 620-1067, 620-1173, 620-1191, 620-1212, 620-1216, 620-1235, 620-1291, 620-1300, 620-1307, 620-1311, 620-1340, 620-1414, 620-1430, |
| | 622-1245, 622-1253, 622-1337, 631-1294, 692-1430, 707-1430, 763-1430, 778-1430, 779-1430, 780-1430, 798-1430, 801-1430, |
| | 813-1430, 822-1430, 853-1421, 853-1430, 862-1430, 867-1430, 874-1430, 895-1430, 899-1430, 909-1430, 923-1430, 924-1430, 926-1430, |
| | 932-1430, 936-1430, 968-1430, 992-1430, 1002-1430, 1006-1430, 1023-1430, 1028-1430, 1042-1430, 1045-1430, 1390-1663, |
| | 2163-2694, 2636-2871, 2670-3149, 2952-3582, 2979-3671, 2995-3634, 3001-3481, 3031-3642, 3044-3736, 3080-3806, 3091-3924, |
| | 3094-3195, 3094-3390, 3094-3618, 3094-3656, 3106-3382, 3106-3503, 3106-3566, 3106-3582, 3106-3619, 3106-3624, 3106-3658, |
| | 3106-3707, 3106-3725, 3106-3757, 3110-3468, 3149-3686, 3172-3835, 3184-3975, 3187-3725, 3198-3801, 3223-3928, 3243-3819, |
| | 3254-3782, 3263-3810, 3270-3789, |
| | 3276-3848, 3294-4153, 3305-4036, 3323-4012, 3333-4001, 3360-4003, 3430-3883, 3467-4262, 3472-3730, 3496-4262, 3500-3813, |
| | 3505-4228, 3526-4115, 3527-3981, 3562-4043, 3564-4266, 3594-3858, 3595-4216, 3595-4228, 3595-4266, 3597-4262, 3603-4266, |
| | 3608-4257, 3612-4266, 3621-4094, 3626-3989, 3629-3894, 3629-4080, 3629-4081, 3629-4203, 3629-4249, 3631-4076, 3634-4387, |
| | 3636-4266, 3652-3841, 3662-4312, 3663-4199, 3663-4257, 3663-4266, 3664-4263, 3671-4266, 3680-4430, 3683-3906, 3683-3928, |
| | 3683-4191, 3683-4314, 3683-4395, 3683-4424, 3683-4428, 3683-4432, 3683-4475, 3683-4485, 3683-4489, 3689-4266, 3693-4294, |
| | 3693-4340, 3697-4266, 3707-4266, 3720-4290, 3728-4041, 3730-4387, 3741-4467, 3746-4472, 3779-4404, 3784-4007, 3816-4118, |
| | 3829-4443, 3830-4422, 3857-4151, 3869-4266, 3938-4264, 3938-4512, 3944-4266, 3959-4512, 3963-4236, 3977-4227, 3977-4229, |
| | 3984-4512, 3985-4512, 3986-4228, 3997-4512, 4001-4306, 4003-4512, 4006-4255, 4021-4058, 4024-4512, 4025-4266, 4031-4266, |
| | 4035-4269, 4039-4512, 4051-4242, 4051-4314, 4051-4512, |
| | 4068-4341, 4077-4512, 4084-4126, 4089-4354, 4097-4478, 4098-4512, 4101-4512, 4139-4512, 4140-4457, 4159-4512, 4193-4512, |
| | 4197-4459, 4197-4468, 4332-4512 |
| 33/7513496CB1/1511 | 1-233, 2-1475, 44-288, 45-346, 46-233, 89-608, 95-367, 106-232, 253-508, 268-500, 296-602, 298-977, 301-738, 301-769, 305-834, |
| | 314-1001, 330-577, 331-930, 338-789, 350-911, 355-564, 355-787, 369-602, 369-636, 413-703, 413-789, 418-529, 419-678, 419-706, |
| | 430-866, 465-852, 465-939, 482-733, 512-759, 528-1034, 531-1113, 546-765, 547-1090, 549-847, 558-820, 559-725, 562-1024, 563-1236, |
| | 585-828, 585-1058, 594-1126, 597-868, 602-1005, 617-737, 620-829, 636-838, 695-1316, 696-1400, 701-997, 704-1286, 705-997, |
| | 708-882, 714-1285, 716-1020, 716-1285, 728-986, 728-1256, 742-1263, 743-1016, 775-1041, 781-1263, 784-1304, 786-897, 796-1060, |
| | 811-1385, 826-1442, 836-978, 836-1095, 836-1111, 841-966, 843-1089, 847-1442, 863-1092, 881-1146, 882-1113, 900-1129, |
| | 915-1183, 920-1178, 922-1173, 939-1442, 946-1193, 946-1364, 946-1382, 952-1195, 961-1226, 961-1343, 967-1151, 967-1230, 968-1153, |
| | 975-1234, 975-1435, 977-1291, 983-1229, 1005-1253, 1005-1260, 1009-1248, 1025-1233, 1035-1312, 1088-1419, 1093-1390, |
| | 1093-1422, 1098-1378, |
| | 1108-1511, 1125-1367, 1131-1382, 1133-1435, 1134-1435, 1147-1378, 1157-1395, 1182-1380, 1201-1475, 1208-1432, 1221-1435, |
| | 1222-1363, 1253-1472, 1258-1435, 1265-1435, 1271-1435, 1313-1511, 1316-1434, 1320-1442, 1323-1442, 1327-1442 |
| 34/7514724CB1/709 | 1-545, 2-709 |
| 35/7514797CB1/969 | 1-606, 1-621, 1-622, 1-623, 1-635, 1-679, 1-681, 1-689, 1-698, 1-702, 1-706, 1-851, 2-968, 58-969 |
| 36/7512100CB1/1102 | 1-843, 271-1102 |
| 37/7512101CB1/1143 | 1-876, 389-1143 |
| 38/7516771CB1/1329 | 1-843, 2-776, 618-1329 |
| 39/7512128CB1/2249 | 1-481, 1-715, 2-488, 471-1380, 476-1331, 695-1583, 1381-2249 |
| 40/7518098CB1/2057 | 1-121, 1-153, 1-276, 1-356, 1-377, 1-505, 1-2057, 3-569, 29-364, 31-312, 96-276, 124-355, 201-489, 215-513, 225-469, 279-482, 284-521, |
| | 352-1181, 828-1086, 828-1282, 846-1435, 848-1115, 849-1128, 852-1476, 866-1557, 906-1386, 906-1549, 914-1560, 932-1176, |
| | 936-1282, 983-1567, 987-1596, 1014-1677, 1016-1585, 1016-1611, 1063-1399, 1066-1213, 1070-1735, 1083-1693, 1104-1354, 1109-1270, |
| | 1122-1682, 1136-1934, 1137-1552, 1176-1625, 1177-1725, 1183-1625, 1185-1468, 1202-1461, 1211-1478, 1221-1477, 1221-1797, |
| | 1226-1486, 1230-1625, 1231-1625, 1231-1630, 1238-1582, 1240-1792, 1260-1577, 1283-1691, 1297-1924, 1305-1622, 1308-1630, |
| | 1324-1586, 1360-1618, 1370-1643, 1380-1623, 1392-1630, 1399-1692, 1400-1693, 1435-1683, 1435-1860, 1437-2012, 1438-1691, |
| | 1466-1703, 1486-1557, 1486-1602, 1486-1613, 1491-1757, 1515-1742, 1541-1747, 1626-1832, 1660-1938, 1662-1910, 1676-1868 |
| 41/7524729CB1/1329 | 1-234, 1-838, 2-796, 588-1329 |
| 42/7520475CB1/3814 | 1-861, 677-1266, 677-1274, 677-1315, 677-3793, 1183-2009, 1184-2052, 1426-2344, 1615-2324, 1869-2745, 2154-3075, 2241-3076, |
| | 2917-3814 |

TABLE 5

| Polynucleotide | | Representative |
|---|---|---|
| SEQ ID NO: | Incyte Project ID: | Library |
| 22 | 7511098CB1 | TESTTUT02 |
| 24 | 7524271CB1 | LUNGNOT15 |
| 25 | 7513132CB1 | THYMNOR02 |
| 26 | 7513134CB1 | THYMNOR02 |
| 28 | 7751418CB1 | SINTNOR01 |
| 30 | 7513020CB1 | STOMTMR02 |
| 31 | 7513162CB1 | PANCNOT08 |
| 32 | 7513164CB1 | PANCNOT08 |
| 33 | 7513496CB1 | PENITUT01 |
| 40 | 7518098CB1 | BRAUNOR01 |

TABLE 6

| Library | Vector | Library Description |
|---|---|---|
| BRAUNOR01 | pINCY | This random primed library was constructed using RNA isolated from striatum, globus pallidus and posterior putamen |
| | | tissue removed from an 81-year-old Caucasian female who died from a hemorrhage and ruptured thoracic aorta due to |
| | | atherosclerosis. Pathology indicated moderate atherosclerosis involving the internal carotids, bilaterally; microscopic |
| | | infarcts of the frontal cortex and hippocampus, and scattered diffuse amyloid plaques and neurofibrillary tangles, |
| | | consistent with age. Grossly, the leptomeninges showed only mild thickening and hyalinization along the superior |
| | | sagittal sinus. The remainder of the leptomeninges was thin and contained some congested blood vessels. Mild |
| | | atrophy was found mostly in the frontal poles and lobes, and temporal lobes, bilaterally. Microscopically, there |
| | | were pairs of Alzheimer type II astrocytes within the deep layers of the neocortex. There was increased |
| | | satellitosis around neurons in the deep gray matter in the middle frontal cortex. The amygdala contained rare diffuse |
| | | plaques and neurofibrillary tangles. The posterior hippocampus contained a microscopic area of cystic cavitation with |
| | | hemosiderin-laden macrophages surrounded by reactive gliosis. Patient history included sepsis, cholangitis, post-operative |
| | | atelectasis, pneumonia CAD, cardiomegaly due to left ventricular hypertrophy, splenomegaly, arteriolonephrosclerosis, |
| | | nodular colloidal goiter, emphysema, CHF, hypothyroidism, and peripheral vascular disease. |
| LUNGNOT15 | pINCY | Library was constructed using RNA isolated from lung tissue removed from a 69-year-old Caucasian male during a |
| | | segmental lung resection. Pathology for the associated tumor tissue indicated residual grade 3 invasive squamous cell |
| | | carcinoma. Patient history included acute myocardial infarction, prostatic hyperplasia, and malignant skin neoplasm. |
| | | Family history included cerebrovascular disease, type I diabetes, acute myocardial infarction, and |
| | | arteriosclerotic coronary disease. |
| PANCNOT08 | pINCY | Library was constructed using RNA isolated from pancreatic tissue removed from a 65-year-old Caucasian female during |
| | | radical subtotal pancreatectomy. Pathology for the associated tumor tissue indicated an invasive grade 2 adenocarcinoma. |
| | | Patient history included type II diabetes, osteoarthritis, cardiovascular disease, benign neoplasm in the large bowel, and a |
| | | cataract. Previous surgeries included a total splenectomy, cholecystectomy, and abdominal hysterectomy. Family history |
| | | included cardiovascular disease, type II diabetes, and stomach cancer. |
| PENITUT01 | pINCY | Library was constructed using RNA isolated from tumor tissue removed from the penis of a 64-year-old Caucasian male |
| | | during penile amputation. Pathology indicated a fungating invasive grade 4 squamous cell carcinoma involving the inner |
| | | wall of the foreskin and extending onto the glans penis. Patient history included benign neoplasm of the large bowel, |
| | | atherosclerotic coronary artery disease, angina pectoris, gout, and obesity. Family history included malignant pharyngeal |
| | | neoplasm, chronic lymphocytic leukemia, and chronic liver disease. |
| SINTNOR01 | PCDNA2.1 | This random primed library was constructed using RNA isolated from small intestine tissue removed from a 31-year-old |
| | | Caucasian female during Roux-en-Y gastric bypass. Patient history included clinical obesity. |
| STOMTMR02 | PCDNA2.1 | This random primed library was constructed using RNA isolated from diseased stomach tissue removed from a |
| | | 76-year-old Caucasian male during proximal gastrectomy and partial esophagectomy. Pathology indicated chronic |
| | | gastritis. Pathology for the matched tumor tissue indicated invasive grade 3 adenocarcinoma forming an |
| | | ulcerated mass at the gastro-esophageal junction. The tumor infiltrated through the muscularis propria into |
| | | the periesophageal adipose tissue. One of four perigastric lymph nodes was positive for tumor. Patient history |
| | | included dysphagia, atherosclerotic coronary artery disease, malignant melanoma of the skin, COPD, benign |
| | | neoplasm of the large bowel, malignant neoplasm of upper lobe of lung, and alcohol abuse. Family history |
| | | included atherosclerotic coronary artery disease and myocardial infarction. |
| TESTTUT02 | pINCY | Library was constructed using RNA isolated from testicular tumor removed from a 31-year-old Caucasian male during |
| | | unilateral orchiectomy. Pathology indicated embryonal carcinoma. |
| THYMNOR02 | pINCY | The library was constructed using RNA isolated from thymus tissue removed from a 2-year-old Caucasian |
| | | female during a thymectomy and patch closure of left atrioventricular fistula. Pathology indicated there was no |
| | | gross abnormality of the thymus. The patient presented with congenital heart abnormalities. Patient history |
| | | included double inlet left ventricle and a rudimentary right ventricle, pulmonary hypertension, cyanosis, |
| | | subaortic stenosis, seizures, and a fracture of the skull base. Family history included reflux neuropathy. |

TABLE 7

| Program | Description | Reference | Parameter Threshold |
|---|---|---|---|
| ABI | A program that removes vector sequences and | Applied Biosystems, Foster City, CA. | |
| FACTURA | masks ambiguous bases in nucleic acid sequences. | | |
| ABI/ | A Fast Data Finder useful in comparing and | Applied Biosystems, Foster City, CA; | Mismatch < 50% |
| PARACEL | annotating amino acid or nucleic acid sequences. | Paracel Inc., Pasadena, CA. | |
| FDF | | | |
| ABI | A program that assembles nucleic acid sequences. | Applied Biosystems, Foster City, CA. | |
| AutoAssembler | | | |
| BLAST | A Basic Local Alignment Search Tool useful in | Altschul, S. F. et al. (1990) J. Mol. Biol. | ESTs: Probability value = |
| | sequence similarity search for amino acid | 215: 403-410; Altschul, S. F. et al. (1997) | 1.0E−8 or less; Full Length |
| | and nucleic acid sequences. BLAST includes | Nucleic Acids Res. 25: 3389-3402. | sequences: Probability value = |
| | five functions: blastp, blastn, blastx, | | 1.0E−10 or less |
| | tblastn, and tblastx. | | |
| FASTA | A Pearson and Lipman algorithm that searches for | Pearson, W. R. and D. J. Lipman (1988) Proc. | ESTs: fasta E value = 1.06E−6; |
| | similarity between a query sequence and a | Natl. Acad Sci. USA 85: 2444-2448; Pearson, | Assembled ESTs: fasta |
| | group of sequences of the same type. FASTA | W. R. (1990) Methods Enzymol. 183: 63-98; | Identity = 95% or greater and |
| | comprises as least five functions: fasta, | and Smith, T. F. and M. S. Waterman (1981) | Match length = 200 bases or |
| | tfasta, fastx, tfastx, and ssearch. | Adv. Appl. Math. 2: 482-489. | greater; fastx E value = |
| | | | 1.0E−8 or less; |
| | | | Full Length sequences: fastx |
| | | | score = 100 or greater |
| BLIMPS | A BLocks IMProved Searcher that matches a | Henikoff, S. and J. G. Henikoff (1991) | Probability value = 1.0E−3 or |
| | sequence against those in BLOCKS, PRINTS, | Nucleic Acids Res. 19: 6565-6572; Henikoff, | less |
| | DOMO, PRODOM, and PFAM databases to | J. G. and S. Henikoff (1996) Methods | |
| | search for gene families, sequence homology, | Enzymol. 266: 88-105; and Attwood, T. K. et | |
| | and structural fingerprint regions. | al. (1997) J. Chem. Inf. Comput. Sci. 37: | |
| | | 417-424. | |
| HMMER | An algorithm for searching a query sequence | Krogh, A. et al. (1994) J. Mol. Biol. | PFAM, INCY, SMART or |
| | against hidden Markov model (HMM)-based | 235: 1501-1531; Sonnhammer, E. L. L. et al. | TIGRFAM hits: Probability |
| | databases of protein family consensus | (1988) Nucleic Acids Res. 26: 320-322; | value = 1.0E−3 or less; Signal |
| | INCY, SMART and TIGRFAM. | Durbin, R. et al. (1998) Our World View, in | peptide hits: Score = 0 or greater |
| | | a Nutshell, Cambridge Univ. Press, pp. 1-350. | |
| ProfileScan | An algorithm that searches for structural and | Gribskov, M. et al. (1988) CABIOS 4: 61-66; | Normalized quality score ≧ |
| | sequence motifs in protein sequences that match | Gribskov, M. et al. (1989) Methods | GCG specified “HIGH” value |
| | sequence patterns defined in Prosite. | Enzymol. 183: 146-159; Bairoch, A. et al. | for that particular Prosite motif. |
| | | (1997) Nucleic Acids Res. 25: 217-221. | Generally, score = 1.4-2.1. |
| Phred | A base-calling algorithm that examines automated | Ewing, B. et al. (1998) Genome Res. 8: 175-185; | |
| | sequencer traces with high sensitivity | Ewing, B. and P. Green (1998) Genome | |
| | and probability. | Res. 8: 186-194. | |
| Phrap | A Phils Revised Assembly Program including | Smith, T. F. and M. S. Waterman (1981) Adv. | Score = 120 or greater; Match |
| | SWAT and CrossMatch, programs based on | Appl. Math. 2: 482-489; Smith, T. F. and | length = 56 or greater |
| | efficient implementation of the Smith- | M. S. Waterman (1981) J. Mol. Biol. 147: | |
| | Waterman algorithm, useful in searching | 195-197; and Green, P., University of | |
| | sequence homology and assembling DNA | Washington, Seattle, WA. | |
| | sequences. | | |
| Consed | A graphical tool for viewing and editing Phrap | Gordon, D. et al. (1998) Genome Res. 8: | |
| | assemblies. | 195-202. | |
| SPScan | A weight matrix analysis program that | Nielson, H. et al. (1997) Protein Engineering | Score = 3.5 or greater |
| | scans protein sequences for the presence | 10: 1-6; Claverie, J. M. and S. Audic (1997) | |
| | of secretory signal peptides. | CABIOS 12: 431-439. | |
| TMAP | A program that uses weight matrices to delineate | Persson, B. and P. Argos (1994) J. Mol. Biol. | |
| | transmembrane segments on protein | 237: 182-192; Persson, B. and P. Argos | |
| | sequences and determine orientation. | (1996) Protein Sci. 5: 363-371. | |
| TMHMMER | A program that uses a hidden Markov model | Sonnhammer, E. L. et al. (1998) Proc. Sixth | |
| | (HMM) to delineate transmembrane segments on | Intl. Conf. On Intelligent Systems for Mol. | |
| | protein sequences and determine orientation. | Biol., Glasgow et al., eds., The Am. Assoc. | |
| | | for Artificial Intelligence (AAAI) Press, | |
| | | Menlo Park, CA, and MIT Press, Cambridge, | |
| | | MA, pp. 175-182. | |
| Motifs | A program that searches amino acid sequences for | Bairoch, A. et al. (1997) Nucleic Acids Res. | |
| | patterns that matched those defined in Prosite. | 25: 217-221; Wisconsin Package Program | |
| | | Manual, version 9, page M51-59, Genetics | |
| | | Computer Group, Madison, WI. | |

TABLE 8

| | | | | | | | | | | | | Asian | |
|---|---|---|---|---|---|---|---|---|---|---|---|---|---|
| SEQ | | | | | | | | | | Caucasian | African | Allele 1 | Hispanic |
| ID | | | | EST | CB1 | EST | | | Amino | Allele 1 | Allele 1 | fre- | Allele 1 |
| NO: | PID | EST ID | SNP ID | SNP | SNP | Allele | Allele 1 | Allele 2 | Acid | frequency | frequency | quency | frequency |
| 22 | 7511098 | 1613409H1 | SNP00047746 | 26 | 179 | C | C | T | noncoding | n/a | n/a | n/a | n/a |
| 22 | 7511098 | 1613409H1 | SNP00135962 | 175 | 328 | T | T | C | L26 | n/a | n/a | n/a | n/a |
| 24 | 7524271 | 1235654H1 | SNP00061841 | 22 | 579 | T | T | C | V193 | n/a | n/a | n/a | n/a |
| 24 | 7524271 | 1235654H1 | SNP00128273 | 147 | 704 | C | C | T | T235 | n/a | n/a | n/a | n/a |
| 24 | 7524271 | 1239659H1 | SNP00111833 | 99 | 142 | C | C | T | Q48 | n/d | n/d | n/d | n/d |
| 24 | 7524271 | 1239659H1 | SNP00131407 | 133 | 176 | T | T | C | V59 | n/a | n/a | n/a | n/a |
| 24 | 7524271 | 1239659H1 | SNP00131408 | 153 | 196 | G | G | A | D66 | n/a | n/a | n/a | n/a |
| 24 | 7524271 | 1988674T6 | SNP00128273 | 87 | 712 | C | C | T | R238 | n/a | n/a | n/a | n/a |
| 24 | 7524271 | 3906011H1 | SNP00061841 | 118 | 592 | T | T | C | Y198 | n/a | n/a | n/a | n/a |
| 24 | 7524271 | 3908459H1 | SNP00061841 | 104 | 594 | T | T | C | D198 | n/a | n/a | n/a | n/a |
| 24 | 7524271 | 5756027H1 | SNP00128273 | 266 | 709 | C | C | T | P237 | n/a | n/a | n/a | n/a |
| 25 | 7513132 | 1232269H1 | SNP00123865 | 164 | 4482 | C | C | G | noncoding | n/a | n/a | n/a | n/a |
| 25 | 7513132 | 1236634F6 | SNP00123863 | 130 | 3002 | C | C | T | noncoding | n/d | n/d | n/d | n/d |
| 25 | 7513132 | 2673666H1 | SNP00123864 | 49 | 3628 | T | T | C | noncoding | n/d | n/d | n/d | n/d |
| 25 | 7513132 | 5507189H1 | SNP00123861 | 24 | 1451 | A | A | G | K458 | n/a | n/a | n/a | n/a |
| 25 | 7513132 | 6769510J1 | SNP00024778 | 38 | 2516 | T | C | T | I813 | n/a | n/a | n/a | n/a |
| 25 | 7513132 | 6823818H1 | SNP00123860 | 435 | 913 | A | A | G | S279 | 0.85 | n/d | n/d | 0.87 |
| 25 | 7513132 | 7730917J1 | SNP00123864 | 197 | 3608 | T | T | C | noncoding | n/d | n/d | n/d | n/d |
| 26 | 7513134 | 1232269H1 | SNP00123865 | 164 | 4343 | C | C | G | noncoding | n/a | n/a | n/a | n/a |
| 26 | 7513134 | 1236634F6 | SNP00123863 | 130 | 3051 | C | C | T | Y991 | n/d | n/d | n/d | n/d |
| 26 | 7513134 | 2673666H1 | SNP00123864 | 49 | 3677 | T | T | C | L1200 | n/d | n/d | n/d | n/d |
| 26 | 7513134 | 5507189H1 | SNP00123861 | 24 | 1451 | A | A | G | K458 | n/a | n/a | n/a | n/a |
| 26 | 7513134 | 6769510J1 | SNP00024778 | 38 | 2516 | T | C | T | I813 | n/a | n/a | n/a | n/a |
| 26 | 7513134 | 6823818H1 | SNP00123860 | 435 | 913 | A | A | G | S279 | 0.85 | n/d | n/d | 0.87 |
| 26 | 7513134 | 7730917J1 | SNP00123864 | 197 | 3657 | T | T | C | N1193 | n/d | n/d | n/d | n/d |
| 27 | 7523653 | 1209901H1 | SNP00001818 | 176 | 1099 | G | A | G | V362 | n/a | n/a | n/a | n/a |
| 27 | 7523653 | 1562015T6 | SNP00047627 | 185 | 1238 | A | A | G | Q408 | 0.39 | n/a | n/a | n/a |
| 27 | 7523653 | 1577631T6 | SNP00113255 | 330 | 714 | C | C | T | S233 | n/d | n/a | n/a | n/a |
| 27 | 7523653 | 5464717H1 | SNP00113255 | 72 | 686 | C | C | T | S224 | n/d | n/a | n/a | n/a |
| 28 | 7751418 | 4250363F6 | SNP00122134 | 60 | 1898 | C | C | T | R584 | n/a | n/a | n/a | n/a |
| 28 | 7751418 | 6801718J1 | SNP00122135 | 251 | 3011 | T | T | C | P955 | 0.09 | n/d | n/d | n/a |
| 28 | 7751418 | 7377634H1 | SNP00122134 | 452 | 1899 | C | C | T | P585 | n/a | n/a | n/a | n/a |
| 28 | 7751418 | 7385118H1 | SNP00102688 | 396 | 535 | T | G | T | I130 | n/a | n/a | n/a | n/a |
| 28 | 7751418 | 7696558J1 | SNP00130108 | 112 | 304 | T | T | C | L53 | n/a | n/a | n/a | n/a |
| 29 | 7523952 | 077214H1 | SNP00108058 | 50 | 1097 | C | C | T | noncoding | n/a | n/a | n/a | n/a |
| 29 | 7523952 | 2851167H1 | SNP000139014 | 209 | 257 | G | G | A | P52 | n/a | n/a | n/a | n/a |
| 29 | 7523952 | 702938H1 | SNP00108057 | 97 | 881 | C | C | T | noncoding | n/a | n/a | n/a | n/a |
| 30 | 7513020 | 2471503H1 | SNP00002541 | 113 | 112 | C | C | T | noncoding | n/a | n/a | n/a | n/a |
| 30 | 7513020 | 3232535H1 | SNP00002542 | 117 | 887 | G | G | A | E244 | n/a | n/a | n/a | n/a |
| 33 | 7513496 | 1451031F6 | SNP00045840 | 40 | 985 | A | G | A | H266 | n/d | n/a | n/a | n/a |
| 33 | 7513496 | 1451031F6 | SNP00061060 | 298 | 1243 | G | G | C | G352 | n/d | n/d | n/d | n/d |
| 33 | 7513496 | 2496437H1 | SNP00061060 | 109 | 1241 | G | G | C | E351 | n/d | n/d | n/d | n/d |
| 33 | 7513496 | 3905291H1 | SNP00045840 | 19 | 990 | G | G | A | V268 | n/d | n/a | n/a | n/a |
| 33 | 7513496 | 3905391H1 | SNP00045840 | 19 | 987 | G | G | A | A267 | n/d | n/a | n/a | n/a |
| 35 | 7514797 | 028080H1 | SNP00148876 | 88 | 132 | C | C | T | L31 | n/a | n/a | n/a | n/a |
| 35 | 7514797 | 1288316H1 | SNP00142504 | 130 | 155 | A | A | G | A38 | n/a | n/a | n/a | n/a |
| 35 | 7514797 | 1306041H1 | SNP00148436 | 32 | 40 | C | A | C | noncoding | n/a | n/a | n/a | n/a |
| 35 | 7514797 | 1360925H1 | SNP00122773 | 100 | 690 | G | G | A | G217 | n/a | n/a | n/a | n/a |
| 35 | 7514797 | 1530917H1 | SNP00013183 | 58 | 416 | A | G | A | S125 | n/a | n/a | n/a | n/a |
| 35 | 7514797 | 1961134H1 | SNP00047253 | 73 | 163 | A | C | A | E41 | n/a | n/a | n/a | n/a |
| 35 | 7514797 | 2924505H1 | SNP00066362 | 74 | 423 | T | C | T | L128 | n/a | n/a | n/a | n/a |
| 36 | 7512100 | 2170258H1 | SNP00105573 | 74 | 122 | T | G | T | noncoding | n/a | n/a | n/a | n/a |
| 36 | 7512100 | 2170258H1 | SNP00105574 | 118 | 166 | T | T | C | noncoding | n/d | n/a | n/a | n/a |
| 37 | 7512101 | 2170258H1 | SNP00105573 | 74 | 74 | T | G | T | noncoding | n/a | n/a | n/a | n/a |
| 37 | 7512101 | 2170258H1 | SNP00105574 | 118 | 118 | T | T | C | noncoding | n/d | n/a | n/a | n/a |
| 38 | 7516771 | 1271895H1 | SNP00003162 | 156 | 94 | A | A | C | T16 | n/a | n/a | n/a | n/a |
| 38 | 7516771 | 1271895H1 | SNP00003163 | 177 | 115 | G | G | A | G23 | n/a | n/a | n/a | n/a |
| 38 | 7516771 | 1271895H1 | SNP00126000 | 76 | 14 | G | G | A | noncoding | n/a | n/a | n/a | n/a |
| 38 | 7516771 | 1524345F6 | SNP00126001 | 120 | 505 | T | T | C | S153 | n/a | n/a | n/a | n/a |
| 39 | 7512128 | 1472714H1 | SNP00063497 | 95 | 1387 | A | G | A | noncoding | 0.32 | n/a | n/a | n/a |
| 40 | 7518098 | 2612308F6 | SNP00124719 | 311 | 1745 | C | C | T | noncoding | n/a | n/a | n/a | n/a |
| 40 | 7518098 | 2636906H1 | SNP00062784 | 237 | 1058 | C | A | C | noncoding | n/a | n/a | n/a | n/a |
| 40 | 7518098 | 2779542F6 | SNP00124718 | 103 | 1323 | A | A | G | noncoding | n/a | n/a | n/a | n/a |
| 40 | 7518098 | 2779542F6 | SNP00124719 | 524 | 1744 | C | C | T | noncoding | n/a | n/a | n/a | n/a |
| 40 | 7518098 | 2845102F6 | SNP00034139 | 349 | 2002 | C | C | T | noncoding | n/a | n/a | n/a | n/a |
| 40 | 7518098 | 3476130H1 | SNP00062783 | 99 | 131 | G | G | C | noncoding | 0.6 | 0.27 | 0.59 | 0.73 |
| 40 | 7518098 | 7653250H1 | SNP00124718 | 371 | 1324 | G | A | G | noncoding | n/a | n/a | n/a | n/a |
| 40 | 7518098 | 8529627H1 | SNP00062783 | 173 | 151 | G | G | C | D4 | 0.6 | 0.27 | 0.59 | 0.73 |
| 41 | 7524729 | 7641577J1 | SNP00140496 | 492 | 573 | A | A | G | K190 | n/a | n/a | n/a | n/a |
| 42 | 7520475 | 3250819H1 | SNP00057803 | 81 | 2755 | T | T | C | S747 | n/a | n/a | n/a | n/a |

1-97. (canceled)

98. An isolated polypeptide selected from the group consisting of:
(a) a polypeptide comprising an amino acid sequence selected from the group consisting of SEQ ID NO: 6 and SEQ ID NO: 8;
(b) a biologically active fragment of the polypeptide of (a); and
(c) an immunogenic fragment of the polypeptide of (a).

99. An isolated polypeptide of claim 98 consisting of the polypeptide of (a), wherein the polypeptide comprises the amino acid sequence of SEQ ID NO: 6.

100. An isolated polypeptide of claim 98 consisting of the polypeptide of (a), wherein the polypeptide comprises the amino acid sequence of SEQ ID NO: 8.

101. An isolated polypeptide of claim 98 consisting of a biologically active fragment of the polypeptide of (a).

102. An isolated polypeptide of claim 98 consisting of an immunogenic fragment of the polypeptide of (a).

103. An isolated polypeptide of claim 98 encoded by a polynucleotide selected from the group consisting of:
(i) a polynucleotide comprising a polynucleotide sequence of SEQ ID NO: 27;
(ii) a polynucleotide comprising a naturally occurring polynucleotide sequence at least 90% identical to SEQ ID NO: 27;
(iii) a polynucleotide comprising a portion of the polynucleotide sequence of SEQ ID NO: 27 that specifically identifies SEQ ID NO: 27.
(iv) a polynucleotide comprising a polynucleotide complementary to the polynucleotide of (i), (ii), or (iii);
(v) an RNA equivalent of the polynucleotide of (i), (ii), (iii) or (iv);
(vi) a polynucleotide of (i), (ii) or (iii) further comprising a promoter sequence operably linked to said polynucleotide of (i), (ii) or (iii).

104. An isolated polypeptide of claim 98 encoded by a polynucleotide selected from the group consisting of:
(i) a polynucleotide comprising a polynucleotide sequence of SEQ ID NO: 29;
(ii) a polynucleotide comprising a naturally occurring polynucleotide sequence at least 90% identical to SEQ ID NO: 29;
(iii) a polynucleotide comprising a portion of the polynucleotide sequence of SEQ ID NO: 29 that specifically identifies SEQ ID NO: 29.
(iv) a polynucleotide comprising a polynucleotide complementary to the polynucleotide of (i), (ii), or (iii);
(v) an RNA equivalent of the polynucleotide of (i), (ii), (iii) or (iv);
(vi) a polynucleotide of (i), (ii) or (iii) further comprising a promoter sequence operably linked to said polynucleotide of (i), (ii) or (iii).

105. An isolated polypeptide of claim 98 produced recombinantly.

106. An isolated polypeptide of claim 103 produced by culturing a cell transformed with a polynucleotide of (iv) under conditions suitable for expression of the polypeptide, and recovering the polypeptide so expressed.

107. An isolated polypeptide of claim 104 produced by culturing a cell transformed with a polynucleotide of (iv) under conditions suitable for expression of the polypeptide, and recovering the polypeptide so expressed.

108. An isolated antibody that specifically binds to a polypeptide of claim 98.

109. An isolated antibody of claim 108, wherein said antibody is selected from the group consisting of a polyclonal antibody, a monoclonal antibody, a chimeric antibody, a single chain antibody, a Fab fragment, a F(ab′)₂ fragment, and a humanized antibody.

110. An isolated antibody of claim 108, wherein said antibody is selected by screening a recombinant immunoglobulin library.

111. An isolated antibody of claim 108, wherein said antibody is selected by screening a Fab expression library.

112. An isolated antibody that specifically binds to a polypeptide of claim 103.

113. An isolated antibody that specifically binds to a polypeptide of claim 104.

114. An isolated antibody of claim 112, wherein said antibody is selected from the group consisting of a polyclonal antibody, a monoclonal antibody, a chimeric antibody, a single chain antibody, a Fab fragment, a F(ab′)₂ fragment, and a humanized antibody.

115. An isolated antibody of claim 113, wherein said antibody is selected from the group consisting of a polyclonal antibody, a monoclonal antibody, a chimeric antibody, a single chain antibody, a Fab fragment, a F(ab′)₂ fragment, and a humanized antibody.

116. A method of detecting a polypeptide of interest in a sample, comprising:
incubating the sample with an antibody that specifically binds to a polypeptide of claim 98 under conditions suitable for binding of the antibody to the polypeptide of interest if present in the sample; and
detecting biding of the polypeptide of interest to the antibody, wherein binding indicates the presence or amount of the polypeptide of interest in the sample.

117. A method of claim 116, wherein the sample is a body fluid sample from a human.

118. An isolated polynucleotide selected from the group consisting of:
(i) a polynucleotide comprising a polynucleotide sequence of SEQ ID NO: 27;
(ii) a polynucleotide comprising a naturally occurring polynucleotide sequence at least 90% identical to SEQ ID NO: 27;
(iii) a polynucleotide comprising a portion of the polynucleotide sequence of SEQ ID NO: 27 that specifically identifies SEQ ID NO: 27.
(iv) a polynucleotide comprising a polynucleotide complementary to the polynucleotide of (i), (ii), or (iii);
(v) an RNA equivalent of the polynucleotide of (i), (ii), (iii) or (iv);
(vi) a polynucleotide of (i), (ii) or (iii) further comprising a promoter sequence operably linked to said polynucleotide of (i), (ii) or (iii).

119. An isolated polynucleotide selected from the group consisting of:
(i) a polynucleotide comprising a polynucleotide sequence of SEQ ID NO: 29;
(ii) a polynucleotide comprising a naturally occurring polynucleotide sequence at least 90% identical to SEQ ID NO: 29;
(iii) a polynucleotide comprising a portion of the polynucleotide sequence of SEQ ID NO: 29 that specifically identifies SEQ ID NO: 29.
(iv) a polynucleotide comprising a polynucleotide complementary to the polynucleotide of (i), (ii), or (iii);
(v) an RNA equivalent of the polynucleotide of (i), (ii), (iii) or (iv);
(vi) a polynucleotide of (i), (ii) or (iii) further comprising a promoter sequence operably linked to said polynucleotide of (i), (ii) or (iii).
